SEQUENCE LISTING (21 sequences)

SEQ ID NO: 1 - protein, 10 aa, synthetic construct
GYTFTHYGMN

SEQ ID NO: 2 - protein, 17 aa, synthetic construct
WVNTYTGEPTYADDFKG

SEQ ID NO: 3 - protein, 9 aa, synthetic construct
EGEGLGFGD

SEQ ID NO: 4 - protein, 16 aa, synthetic construct
RSSQSIVHSHGDTYLE

SEQ ID NO: 5 - protein, 7 aa, synthetic construct
KVSNRFS

SEQ ID NO: 6 - protein, 9 aa, synthetic construct
FQGSHIPVT

SEQ ID NO: 7 - protein, 118 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSS

SEQ ID NO: 8 - protein, 112 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIK

SEQ ID NO: 9 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 10 aa, synthetic construct
GFNIKNTYMH

SEQ ID NO: 12 - protein, 17 aa, synthetic construct
RIDPAKGHTEYAPKFLG

SEQ ID NO: 13 - protein, 11 aa, Synthetic construct
HASQDISDNIG

SEQ ID NO: 14 - protein, 7 aa, synthetic construct
HGTNLED

SEQ ID NO: 15 - protein, 9 aa, synthetic construct
VQYAQFPWT

SEQ ID NO: 16 - protein, 112 aa, synthetic construct
EVQLVQSGAEVKKPGSSVKVSCKASGFNIKNTYMHWVRQAPGQGLEWIGRIDPAKGHTEYAPKFLGRVTITADESTNTAYMELSSLRSEDTAVYYCYYVDVWGQGTTVTVSS

SEQ ID NO: 17 - protein, 107 aa, synthetic construct
DIQMTQSPSSLSASVGDRVTITCHASQDISDNIGWLQQKPGKSFKLLIYHGTNLEDGVPSRFSGSGSGTDYTLTISSLQPEDFATYYCVQYAQFPWTFGGGTKVEIK

SEQ ID NO: 18 - protein, 442 aa, synthetic construct
EVQLVQSGAEVKKPGSSVKVSCKASGFNIKNTYMHWVRQAPGQGLEWIGRIDPAKGHTEYAPKFLGRVTITADESTNTAYMELSSLRSEDTAVYYCYYVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 214 aa, synthetic construct
DIQMTQSPSSLSASVGDRVTITCHASQDISDNIGWLQQKPGKSFKLLIYHGTNLEDGVPSRFSGSGSGTDYTLTISSLQPEDFATYYCVQYAQFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 447 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 441 aa, synthetic construct
EVQLVQSGAEVKKPGSSVKVSCKASGFNIKNTYMHWVRQAPGQGLEWIGRIDPAKGHTEYAPKFLGRVTITADESTNTAYMELSSLRSEDTAVYYCYYVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0059280 A1
(43) Pub. Date: Feb. 20, 2025

(54) METHODS OF TREATING HUMAN IMMUNODEFICIENCY VIRUS (HIV) DISEASE

(71) Applicant: AbbVie Inc., North Chicago, IL (US)

(72) Inventors: Ana Gabriela Pires dos Santos, Round Lake, IL (US); Preethi Krishnan, Pleasanton, CA (US); Mong-Jen Chen, North Chicago, IL (US); Aline Goebel, North Chicago, IL (US); Neha Thakre, Darmstadt (DE); Insa Winzenborg, Neustadt/Wstr. (DE); Tanaya Vaidya, North Chicago, IL (US)

(21) Appl. No.: 18/806,311

(22) Filed: Aug. 15, 2024

Related U.S. Application Data
(60) Provisional application No. 63/591,332, filed on Oct. 18, 2023.
(60) Provisional application No. 63/519,760, filed on Aug. 15, 2023.

(51) Int. Cl. C07K 16/28; A61K 9/00; A61P 31/18

(52) U.S. Cl. CPC C07K 16/2818; A61K 9/0019; A61P 31/18; C07K 16/2839; A61K 2123/00; C07K 2317/21; C07K 2317/24; C07K 2317/92

(57) ABSTRACT

The invention described herein relates to methods of treating HIV infection comprising administering an anti-PD-1 monoclonal antibody and/or an anti-α4β7 monoclonal antibody.

### RELATED APPLICATIONS

This application claims the benefit of priority of U.S. Provisional Patent Application Ser. No. 63/519,760, filed Aug. 15, 2023, and 63/591,332, filed Oct. 18, 2023, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Oct. 18, 2023, is named ABV21555USL2_ST26.xml and is 21,558 bytes in size.

### FIELD OF THE INVENTION

The present disclosure is directed to methods of treating Human Immunodeficiency Virus (HIV) disease, and in particular, to methods of treating HIV disease using anti-PD-1 antibodies and/or anti-α4β7 antibodies, in patients living with HIV who are virologically suppressed on anti-retroviral therapy (ART) undergoing analytical treatment interruption.

### BACKGROUND OF THE INVENTION

HIV infection is a chronic condition that remains a major global health problem, with an estimated 38 million people world-wide living with the disease in 2020. Anti-retroviral therapy (ART) can suppress HIV replication, significantly decreasing AIDS-related mortality and improving patients' lives. However, these regimens cannot eliminate the HIV DNA integrated in the genome of CD4+ T cells. In addition, a high adherence rate to lifelong daily ART is required to maintain viral suppression in plasma and prevent resistance. Such adherence is challenging for many patients and drug resistance remains a problem.

There remains a significant unmet medical need for treatment regimens of finite duration (as opposed to chronic treatment requiring high adherence for life) that can induce a sustained viral suppression without ART.

### SUMMARY OF THE INVENTION

The present disclosure relates to methods for treating HIV infection in a human subject. In some embodiments, the human subject is living with HIV-1 who is responsive to anti-retroviral therapy, undergoing analytical treatment interruption (ATI) and treated with monoclonal antibodies. In some embodiments, the human subject is any person living with HIV-1 (PLWH). In some embodiments, the human subject is living with HIV-1 who has limited future treatment options.

In one embodiment, the present invention provides a method of treating HIV infection in a patient infected with HIV-1, the method comprising administering to the patient a therapeutically effective amount of a monoclonal antibody which binds to human programmed death receptor 1 (PD-1).

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered at from about 10 mg to about 20 mg.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered at about 10 mg.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein, after an initial dose, the monoclonal antibody which binds to human PD-1 is administered at about 10 mg every 2 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered at about 10 mg every 2 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein, after an initial dose, the monoclonal antibody which binds to human PD-1 is administered at about 10 mg every 2 weeks for a duration of 6 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered at about 10 mg every 2 weeks for a duration of 6 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered at about 10 mg at Week 0 (Day 1), Week 2 (Day 15), Week 4 (Day 29), and Week 6 (Day 43).

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered at about 20 mg.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein, after an initial dose, the monoclonal antibody which binds to human PD-1 is administered at about 20 mg every 2 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered at about 20 mg every 2 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein, after an initial dose, the monoclonal antibody which binds to human PD-1 is administered at about 20 mg every 2 weeks for a duration of 6 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered at about 20 mg every 2 weeks for a duration of 6 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered at about 20 mg at Week 0 (Day 1), Week 2 (Day 15), Week 4 (Day 29), and Week 6 (Day 43).

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody that binds to human PD-1 is administered to the patient via subcutaneous (SC) injection or intravenous (IV) infusion.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered via IV infusion at about 10 mg at Week 0 (Day 1), Week 2 (Day 15), Week 4 (Day 29), and Week 6 (Day 43).

In one embodiment, the present invention provides a method of treating HIV infection in a patient infected with HIV-1, the method comprising: administering to the patient a therapeutically effective amount of a monoclonal antibody which binds to human programmed death receptor 1 (PD-1), wherein the monoclonal antibody which binds to human PD-1 is administered via SC injection at about 20 mg at Week 0 (Day 1), Week 2 (Day 15), Week 4 (Day 29), and Week 6 (Day 43).

In one embodiment, the present invention provides a method of treating HIV infection in a patient infected with HIV-1, the method comprising: administering to the patient a therapeutically effective amount of a monoclonal antibody which binds to human α4β7.

In one embodiment, the present invention provides a method of treating HIV infection in a patient infected with HIV-1, the method comprising administering to the patient a therapeutically effective amount of a monoclonal antibody which binds to human α4β7, wherein the monoclonal antibody which binds to α4β7 is administered at from about 800 mg to about 1600 mg.

In one embodiment, the present invention provides a method of treating HIV infection in a patient infected with HIV-1, the method comprising administering to the patient a therapeutically effective amount of a monoclonal antibody which binds to human α4β7, wherein the monoclonal antibody which binds to α4β7 is administered at about 800 mg.

In one embodiment, the present invention provides a method of treating HIV infection in a patient infected with HIV-1, the method comprising administering to the patient a therapeutically effective amount of a monoclonal antibody which binds to human α4β7, wherein the monoclonal antibody which binds to α4β7 is administered at about 800 mg every 4 weeks.

In one embodiment, the present invention provides a method of treating HIV infection in a patient infected with HIV-1, the method comprising administering to the patient a therapeutically effective amount of a monoclonal antibody which binds to human α4β7, wherein, after an initial dose, the monoclonal antibody which binds to α4β7 is administered at about 800 mg every 4 weeks.

In one embodiment, the present invention provides a method of treating HIV infection in a patient infected with HIV-1, the method comprising: administering to the patient a therapeutically effective amount of a monoclonal antibody which binds to human α4β7, wherein the monoclonal antibody which binds to α4β7 is administered at about 1600 mg.

In one embodiment, the present invention provides a method of treating HIV infection in a patient infected with HIV-1, the method comprising: administering to the patient a therapeutically effective amount of a monoclonal antibody which binds to human α4β7, wherein the monoclonal antibody which binds to α4β7 is administered at about 1600 mg every 4 weeks.

In one embodiment, the present invention provides a method of treating HIV infection in a patient infected with HIV-1, the method comprising: administering to the patient a therapeutically effective amount of a monoclonal antibody which binds to human α4β7, wherein, after an initial dose, the monoclonal antibody which binds to α4β7 is administered at about 1600 mg every 4 weeks.

In one embodiment, the present invention provides a method of treating HIV infection in a patient infected with HIV-1, the method comprising: administering to the patient a therapeutically effective amount of a monoclonal antibody which binds to human α4β7, wherein, after an initial dose, the monoclonal antibody which binds to α4β7 is administered at about 800 mg every 4 weeks for a duration of 8 weeks.

In one embodiment, the present invention provides a method of treating HIV infection in a patient infected with HIV-1, the method comprising: administering to the patient a therapeutically effective amount of a monoclonal antibody which binds to human α4β7, wherein the monoclonal antibody which binds to α4β7 is administered at about 800 mg every 4 weeks for a duration of 8 weeks.

In one embodiment, the present invention provides a method of treating HIV infection in a patient infected with HIV-1, the method comprising: administering to the patient a therapeutically effective amount of a monoclonal antibody which binds to human α4β7, wherein the monoclonal antibody which binds to α4β7 is administered at about 800 mg at Week 0 (Day 1), Week 4 (Day 29), and Week 8 (Day 57).

In one embodiment, the present invention provides a method of treating HIV infection in a patient infected with HIV-1, the method comprising: administering to the patient a therapeutically effective amount of a monoclonal antibody which binds to human α4β7, wherein, after an initial dose, the monoclonal antibody which binds to α4β7 is administered at about 1600 mg every 4 weeks for a duration of 8 weeks.

In one embodiment, the present invention provides a method of treating HIV infection in a patient infected with HIV-1, the method comprising: administering to the patient a therapeutically effective amount of a monoclonal antibody which binds to human α4β7, wherein the monoclonal antibody which binds to α4β7 is administered at about 1600 mg every 4 weeks for a duration of 8 weeks.

In one embodiment, the present invention provides a method of treating HIV infection in a patient infected with HIV-1, the method comprising: administering to the patient a therapeutically effective amount of a monoclonal antibody which binds to human α4β7, wherein the monoclonal antibody which binds to α4β7 is administered at about 1600 mg at Week 0 (Day 1), Week 4 (Day 29), and Week 8 (Day 57).

In one embodiment, the present invention provides a method of treating HIV infection in a patient infected with HIV-1, the method comprising: administering to the patient a therapeutically effective amount of a monoclonal antibody which binds to human α4β7, wherein the monoclonal antibody which binds to α4β7 is administered via subcutaneous (SC) injection or intravenous (IV) infusion.

In one embodiment, the present invention provides a method of treating HIV infection in a patient infected with HIV-1, the method comprising: administering to the patient a therapeutically effective amount of a monoclonal antibody which binds to human α4β7, wherein the monoclonal antibody which binds to α4β7 is administered via IV infusion at about 800 mg at Week 0 (Day 1), Week 4 (Day 29), and Week 8 (Day 57).

In one embodiment, the present invention provides a method of treating HIV infection in a patient infected with HIV-1, the method comprising: administering to the patient a therapeutically effective amount of a monoclonal antibody which binds to human α4β7, wherein the monoclonal antibody which binds to α4β7 is administered via IV infusion at about 1600 mg at Week 0 (Day 1), Week 4 (Day 29), and Week 8 (Day 57).

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7 at the same time.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7 sequentially.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered at about 10 mg and the monoclonal antibody which binds to α4β7 is administered at about 800 mg.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered at about 10 mg and the monoclonal antibody which binds to human α4β7 is administered at about 1600 mg.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein, after initial doses, the monoclonal antibody which binds to human PD-1 is administered for a duration of 6 weeks and the monoclonal antibody which binds to human α4β7 is administered for a duration of 8 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered for a duration of 6 weeks and the monoclonal antibody which binds to human α4β7 is administered for a duration of 8 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered every 2 weeks and the monoclonal antibody which binds to human α4β7 is administered every 4 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein, after initial doses, the monoclonal antibody which binds to human PD-1 is administered every 2 weeks for a duration of 6 weeks and the monoclonal antibody which binds to human α4β7 is administered every 4 weeks for a duration of 8 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered every 2 weeks for a duration of 6 weeks and the monoclonal antibody which binds to human α4β7 is administered every 4 weeks for a duration of 8 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein, after an initial dose, the monoclonal antibody which binds to human PD-1 is administered at about 10 mg every 2 weeks for a duration of 6 weeks and, after an initial dose, the monoclonal antibody which binds to human α4β7 is administered at about 800 mg every 4 weeks for a duration of 8 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered at about 10 mg every 2 weeks for a duration of 6 weeks and the monoclonal antibody which binds to human α4β7 is administered at about 800 mg every 4 weeks for a duration of 8 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered at about 10 mg on Week 0 (Day 1), Week 2 (Day 15), Week 4 (Day 29), and Week 6 (Day 43), and the monoclonal antibody which binds to human α4β7 is administered at about 800 mg on Week 0 (Day 1), Week 4 (Day 29), and Week 8 (Day 57).

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein, after an initial dose, the monoclonal antibody which binds to human PD-1 is administered at about 10 mg every 2 weeks for a duration of 6 weeks and, after an initial dose, the monoclonal antibody which binds to human α4β7 is administered at about 1600 mg every 4 weeks for a duration of 8 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered at about 10 mg every 2 weeks for a duration of 6 weeks and the monoclonal antibody which binds to human α4β7 is administered at about 1600 mg every 4 weeks for a duration of 8 weeks.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered at about 10 mg on Week 0 (Day 1), Week 2 (Day 15), Week 4 (Day 29), and Week 6 (Day 43), and the monoclonal antibody which binds to human α4β7 is administered at about 1600 mg on Week 0 (Day 1), Week 4 (Day 29), and Week 8 (Day 57).

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered via intravenous (IV) infusion or via subcutaneous (SC) injection.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human α4β7 is administered via intravenous (IV) infusion or via subcutaneous (SC) injection.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered via intravenous (IV) infusion or via subcutaneous (SC) injection, and the monoclonal antibody which binds to human α4β7 is administered via intravenous (IV) infusion or via subcutaneous (SC) injection.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered via intravenous (IV) infusion or via subcutaneous (SC) injection, and the monoclonal antibody which binds to human α4β7 is administered via intravenous (IV) infusion.

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered via IV infusion at about 10 mg on Week 0 (Day 1), Week 2 (Day 15), Week 4 (Day 29), and Week 6 (Day 43), and the monoclonal antibody which binds to human α4β7 is administered via IV infusion at about 800 mg on Week 0 (Day 1), Week 4 (Day 29), and Week 8 (Day 57).

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered via IV infusion at about 10 mg on Week 0 (Day 1), Week 2 (Day 15), Week 4 (Day 29), and Week 6 (Day 43), and the monoclonal antibody which binds to human α4β7 is administered via IV infusion at about 1600 mg on Week 0 (Day 1), Week 4 (Day 29), and Week 8 (Day 57).

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered via SC injection at about 20 mg on Week 0 (Day 1), Week 2 (Day 15), Week 4 (Day 29), and Week 6 (Day 43), and the monoclonal antibody which binds to human α4β7 is administered via IV infusion at about 800 mg on Week 0 (Day 1), Week 4 (Day 29), and Week 8 (Day 57).

In one embodiment, the present invention provides a method of treating HIV infection in a patient infected with HIV-1, the method comprising: administering to the patient a therapeutically effective amount of a monoclonal antibody which binds to human programmed death receptor 1 (PD-1) and a monoclonal antibody which binds to human α4β7, wherein the monoclonal antibody which binds to human PD-1 is administered via SC injection at about 20 mg on Week 0 (Day 1), Week 2 (Day 15), Week 4 (Day 29), and Week 6 (Day 43), and the monoclonal antibody which binds to human α4β7 is administered via IV infusion at about 1600 mg on Week 0 (Day 1), Week 4 (Day 29), and Week 8 (Day 57).

In one embodiment, in any one of the methods above, after about 25 weeks the patient achieves an HIV viral load of less than about 5,000 copies/mL.

In another embodiment, in any one of the methods above, the HIV-1 viral load of less than about 5,000 copies/mL is achieved without antiretroviral treatment.

In another embodiment, in any one of the methods above, the HIV-1 viral load of less than 1,000 copies/mL is achieved without antiretroviral treatment.

In another embodiment, in any one of the methods above, the HIV-1 viral load of less than 200 copies/mL is achieved without antiretroviral treatment.

In one embodiment, in any one of the methods above, the patient achieves an HIV viral load of less than about 1,000 copies/mL for at least one year.

In one embodiment, in any one of the methods above, the patient achieves an HIV viral load of less than about 200 copies/mL for at least one year.

In one embodiment, the present invention provides a method of treating HIV infection in a patient infected with HIV-1, the method comprising: administering to the patient a therapeutically effective amount of a monoclonal antibody which binds to human PD-1.

In one embodiment, the present invention provides a method of treating HIV infection in a patient infected with HIV-1, the method comprising: administering to the patient a therapeutically effective amount of a monoclonal antibody which binds to human PD-1; wherein the monoclonal antibody is mAb2 which comprises (i) a VH chain comprising three complementarity-determining regions (CDRs); and (ii) a VL chain comprising three CDRs, wherein:

VH CDR#1 is
(SEQ ID NO: 1)
GYTFTHYGMN;
VH CDR#2 is
(SEQ ID NO: 2)
WVNTYTGEPTYADDFKG;
VH CDR#3 is
(SEQ ID NO: 3)
EGEGLGFGD;
VL CDR#1 is
(SEQ ID NO: 4)
RSSQSIVHSHGDTYLE;
VL CDR#2 is
(SEQ ID NO: 5)
KVSNRFS;
and
VL CDR#3 is
(SEQ ID NO: 6)
FQGSHIPVT.

In another embodiment, in any one of the methods above, the method includes: administering to the patient a therapeutically effective amount of a monoclonal antibody which binds to human PD-1; wherein the monoclonal antibody comprises (i) a VH comprising an amino acid sequence having at least 90%, at least 95%, at least 98%, at least 99%, or 100% identify to the amino acid sequence of SEQ ID NO: 7; and (ii) a VL comprising an amino acid sequence having at least 90%, at least 95%, at least 98%, at least 99%, or 100% identify to the amino acid sequence of SEQ ID NO: 8.

In another embodiment, in any one of the methods above, the method includes: administering to the patient a therapeutically effective amount of a monoclonal antibody which binds to human PD-1; wherein the monoclonal antibody is mAb2 which comprises (i) a VH comprising the amino acid sequence of SEQ ID NO: 7; and (ii) a VL comprising the amino acid sequence of SEQ ID NO: 8.

In another embodiment, in any one of the methods above, the method includes: administering to the patient a therapeutically effective amount of a monoclonal antibody which binds to human PD-1; wherein the monoclonal antibody comprises (i) a heavy chain (HC) comprising an amino acid sequence having at least 90%, at least 95%, at least 98%, at least 99%, or 100% identify to the amino acid sequence of SEQ ID NO: 9; and (ii) a light chain (LC) comprising an amino acid sequence having at least 90%, at least 95%, at least 98%, at least 99%, or 100% identify to the amino acid sequence of SEQ ID NO: 10.

In another embodiment, in any one of the methods above, the method includes: administering to the patient a therapeutically effective amount of a monoclonal antibody which binds to human PD-1; wherein the monoclonal antibody is mAb2 which comprises (i) a heavy chain (HC) comprising the amino acid sequence of SEQ ID NO: 9; and (ii) a light chain (LC) comprising the amino acid sequence of SEQ ID NO: 10.

In another embodiment, in any one of the methods above, the method includes: administering to the patient a therapeutically effective amount of a monoclonal antibody which binds to human PD-1; wherein the monoclonal antibody is mAb2 which comprises (i) a heavy chain (HC) comprising the amino acid sequence of SEQ ID NO: 20; and (ii) a light chain (LC) comprising the amino acid sequence of SEQ ID NO: 10.

In another embodiment, in any one of the methods above the method includes: administering to the patient a therapeutically effective amount of a monoclonal antibody which binds to human PD-1; wherein the monoclonal antibody is mAb2 which comprises (i) two identical heavy chains, each of which comprising the amino acid sequence of SEQ ID NO: 9; and (ii) two identical light chains, each of which comprising the amino acid sequence of SEQ ID NO: 10.

In another embodiment, in any one of the methods above, the method includes: administering to the patient a therapeutically effective amount of a monoclonal antibody which binds to human PD-1; wherein the monoclonal antibody is mAb2 which comprises (i) two identical heavy chains, each of which comprising the amino acid sequence of SEQ ID NO: 20; and (ii) two identical light chains, each of which comprising the amino acid sequence of SEQ ID NO: 10.

In another embodiment, in any one of the methods above, the method includes: administering to the patient a therapeutically effective amount of a monoclonal antibody which binds to human PD-1; wherein the monoclonal antibody is mAb2 which comprises (i) one heavy chain comprising the amino acid sequence of SEQ ID NO: 9; (ii) one heavy chain comprising the amino acid sequence of SEQ ID NO: 20; and (iii) two identical light chains, each of which comprising the amino acid sequence of SEQ ID NO: 10.

In one embodiment, the present invention provides a method of treating HIV infection in a patient infected with HIV-1 according to any one of the methods above, wherein the monoclonal antibody which binds to human PD-1 monoclonal antibody is administered intravenously at about 10 mg or subcutaneously at about 20 mg.

In one embodiment, in any one of the methods above, after about 25 weeks the patient achieves an HIV viral load of less than about 5,000 copies/mL.

In another embodiment, in any one of the methods above, the HIV-1 viral load of less than about 5,000 copies/mL is achieved without antiretroviral treatment.

In another embodiment, in any one of the methods above, the HIV-1 viral load of less than 1,000 copies/mL is achieved without antiretroviral treatment.

In another embodiment, in any one of the methods above, the HIV-1 viral load of less than 200 copies/mL is achieved without antiretroviral treatment.

In one embodiment, in any one of the methods above, the patient achieves an HIV viral load of less than about 1,000 copies/mL for at least one year.

In one embodiment, in any one of the methods above, the patient achieves an HIV viral load of less than about 200 copies/mL for at least one year.

In one embodiment, the present invention provides a method of treating HIV infection in a patient infected with HIV-1, the method comprising: administering to the patient a therapeutically effective amount of a monoclonal antibody which binds to human α4β7.

In another embodiment, in any one of the methods above, the method includes: administering to the patient a therapeutically effective amount of a monoclonal antibody which binds to human α4β7; wherein the monoclonal antibody is mAb1 which comprises (i) a heavy chain variable region (VH) comprising three CDRs: VH CDR #1, VH CDR #2, and VH CDR #3; and (ii) a light chain variable region (VL) comprising three CDRs: VL CDR #1, VL CDR #2, and VL CDR #3, wherein:

VH CDR#1 is
(SEQ ID NO: 11)
GFNIKNTYMH;
VH CDR#2 is
(SEQ ID NO: 12)
RIDPAKGHTEYAPKFLG;
VH CDR#3 is
VDV;
VL CDR#1 is
(SEQ ID NO: 13)
HASQDISDNIG;
VL CDR#2 is
(SEQ ID NO: 14)
HGTNLED;
and
VL CDR#3 is
(SEQ ID NO: 15)
VQYAQFPWT.

In another embodiment, in any one of the methods above, the method includes: administering to the patient a therapeutically effective amount of a monoclonal antibody which binds to human α4β7; wherein the monoclonal antibody comprises (i) a VH comprising an amino acid sequence having at least 90%, at least 95%, at least 98%, at least 99%, or 100% identify to the amino acid sequence of SEQ ID NO: 16; and (ii) a VL comprising an amino acid sequence having at least 90%, at least 95%, at least 98%, at least 99%, or 100% identify to the amino acid sequence of SEQ ID NO: 17.

In another embodiment, in any one of the methods above, the method includes: administering to the patient a therapeutically effective amount of a monoclonal antibody which binds to human α4β7; wherein the monoclonal antibody is mAb1 which comprises (i) a VH comprising the amino acid sequence of SEQ ID NO: 16; and (ii) a VL comprising the amino acid sequence of SEQ ID NO: 17.

In another embodiment, in any one of the methods above, the method includes: administering to the patient a therapeutically effective amount of a monoclonal antibody which binds to human α4β7; wherein the monoclonal antibody comprises (i) a heavy chain (HC) comprising an amino acid sequence having at least 90%, at least 95%, at least 98%, at least 99%, or 100% identify to the amino acid sequence of SEQ ID NO: 18; and (ii) a light chain (LC) comprising an amino acid sequence having at least 90%, at least 95%, at least 98%, at least 99%, or 100% identify to the amino acid sequence of SEQ ID NO: 19.

In another embodiment, in any one of the methods above, the method includes: administering to the patient a therapeutically effective amount of a monoclonal antibody which binds to human α4β7; wherein the monoclonal antibody is mAb1 which comprises (i) a heavy chain (HC) comprising the amino acid sequence of SEQ ID NO: 18; and (ii) a light chain (LC) comprising the amino acid sequence of SEQ ID NO: 19.

In another embodiment, in any one of the methods above, the method includes: administering to the patient a therapeutically effective amount of a monoclonal antibody which binds to human α4β7; wherein the monoclonal antibody is mAb1 which comprises (i) a heavy chain (HC) comprising the amino acid sequence of SEQ ID NO: 21; and (ii) a light chain (LC) comprising the amino acid sequence of SEQ ID NO: 19.

In another embodiment, in any one of the methods above, the method includes: administering to the patient a therapeutically effective amount of a monoclonal antibody which binds to human α4β7; wherein the monoclonal antibody is mAb1 which comprises (i) two identical heavy chains, each of which comprising the amino acid sequence of SEQ ID NO: 18; and (ii) two identical light chains, each of which comprising the amino acid sequence of SEQ ID NO: 19.

In another embodiment, in any one of the methods above, the method includes: administering to the patient a therapeutically effective amount of a monoclonal antibody which binds to human α4β7; wherein the monoclonal antibody is mAb1 which comprises (i) two identical heavy chains, each of which comprising the amino acid sequence of SEQ ID NO: 21; and (ii) two identical light chains, each of which comprising the amino acid sequence of SEQ ID NO: 19.

In another embodiment, in any one of the methods above, the method includes: administering to the patient a therapeutically effective amount of a monoclonal antibody which binds to human α4β7; wherein the monoclonal antibody is mAb1 which comprises (i) one heavy chain comprising the amino acid sequence of SEQ ID NO: 18; (ii) one heavy chain comprising the amino acid sequence of SEQ ID NO: 21; and (iii) two identical light chains, each of which comprising the amino acid sequence of SEQ ID NO: 19.

In another embodiment, in any one of the methods above, the monoclonal antibody which binds to human α4β7 is administered at about 800 mg.

In another embodiment, in any one of the methods above, the monoclonal antibody which binds to human α4β7 is administered at about 1600 mg.

In another embodiment, in any one of the methods above, after about 25 weeks the patient achieves an HIV viral load of less than about 5,000 copies/mL.

In another embodiment, in any one of the methods above, the HIV-1 viral load of less than about 5,000 copies/mL is achieved without antiretroviral treatment.

In another embodiment, in any one of the methods above, the HIV-1 viral load of less than 1,000 copies/mL is achieved without antiretroviral treatment.

In another embodiment, in any one of the methods above, the HIV-1 viral load of less than 200 copies/mL is achieved without antiretroviral treatment.

In one embodiment, in any one of the methods above, the patient achieves an HIV viral load of less than about 1,000 copies/mL for at least one year.

In one embodiment, in any one of the methods above, the patient achieves an HIV viral load of less than about 200 copies/mL for at least one year.

In one embodiment, the present invention provides a method of treating HIV infection in a patient infected with HIV-1, the method comprising: administering to the patient a therapeutically effective amount of a first monoclonal antibody which binds to human PD-1; and administering to the patient a therapeutically effective amount of a second monoclonal antibody which binds to human α4β7.

In another embodiment, in any one of the methods above, the method comprises: administering a first monoclonal antibody which binds to human PD-1; and administering a second monoclonal antibody which binds to α4β7.

In another embodiment, in any one of the methods above, the first monoclonal antibody is mAb2 which comprises (i) a VH chain comprising three CDRs; and (ii) a VL chain comprising three CDRs, wherein:

VH CDR#1 is
(SEQ ID NO: 1)
GYTFTHYGMN;
VH CDR#2 is
(SEQ ID NO: 2)
WVNTYTGEPTYADDFKG;
VH CDR#3 is
(SEQ ID NO: 3)
EGEGLGFGD;
VL CDR#1 is
(SEQ ID NO: 4)
RSSQSIVHSHGDTYLE;
VL CDR#2 is
(SEQ ID NO: 5)
KVSNRFS;
and
VL CDR#3 is
(SEQ ID NO: 6)
FQGSHIPVT.

In another embodiment, in any one of the methods above, the first monoclonal antibody comprises (i) a VH comprising an amino acid sequence having at least 90%, at least 95%, at least 98%, at least 99%, or 100% identify to the amino acid sequence of SEQ ID NO: 7; and (ii) a VL comprising an amino acid sequence having at least 90%, at least 95%, at least 98%, at least 99%, or 100% identify to the amino acid sequence of SEQ ID NO: 8.

In another embodiment, in any one of the methods above, the first monoclonal antibody is mAb2 which comprises (i) a VH comprising the amino acid sequence of SEQ ID NO: 7; and (ii) a VL comprising the amino acid sequence of SEQ ID NO: 8.

In another embodiment, in any one of the methods above, the first monoclonal antibody comprises (i) a heavy chain (HC) comprising an amino acid sequence having at least 90%, at least 95%, at least 98%, at least 99%, or 100% identify to the amino acid sequence of SEQ ID NO: 9; and (ii) a light chain (LC) comprising an amino acid sequence having at least 90%, at least 95%, at least 98%, at least 99%, or 100% identify to the amino acid sequence of SEQ ID NO: 10.

In another embodiment, in any one of the methods above, the first monoclonal antibody is mAb2 which comprises (i) a heavy chain (HC) comprising the amino acid sequence of SEQ ID NO: 9; and (ii) a light chain (LC) comprising the amino acid sequence of SEQ ID NO: 10.

In another embodiment, in any one of the methods above, the first monoclonal antibody is mAb2 which comprises (i) a heavy chain (HC) comprising the amino acid sequence of SEQ ID NO: 20; and (ii) a light chain (LC) comprising the amino acid sequence of SEQ ID NO: 10.

In another embodiment, in any one of the methods above, the first monoclonal antibody is mAb2 which comprises (i) two identical heavy chains, each of which comprising the amino acid sequence of SEQ ID NO: 9; and (ii) two identical light chains, each of which comprising the amino acid sequence of SEQ ID NO: 10.

In another embodiment, in any one of the methods above, the first monoclonal antibody is mAb2 which comprises (i) two identical heavy chains, each of which comprising the amino acid sequence of SEQ ID NO: 20; and (ii) two identical light chains, each of which comprising the amino acid sequence of SEQ ID NO: 10.

In another embodiment, in any one of the methods above, the first monoclonal antibody is mAb2 which comprises (i) one heavy chain comprising the amino acid sequence of SEQ ID NO: 9; (ii) one heavy chain comprising the amino acid sequence of SEQ ID NO: 20; and (iii) two identical light chains, each of which comprising the amino acid sequence of SEQ ID NO: 10.

In another embodiment, in any one of the methods above, the second monoclonal antibody is mAb1 which comprises (i) a VH chain comprising three CDRs; and (ii) a VL chain comprising three CDRs, wherein:

VH CDR#1 is
(SEQ ID NO: 11)
GFNIKNTYMH;
VH CDR#2 is
(SEQ ID NO: 12)
RIDPAKGHTEYAPKFLG;
VH CDR#3 is
VDV;
VL CDR#1 is
(SEQ ID NO: 13)
HASQDISDNIG;
VL CDR#2 is
(SEQ ID NO: 14)
HGTNLED;
and
VL CDR#3 is
(SEQ ID NO: 15)
VQYAQFPWT.

In another embodiment, in any one of the methods above, the second monoclonal antibody comprises (i) a VH comprising an amino acid sequence having at least 90%, at least 95%, at least 98%, at least 99%, or 100% identify to the amino acid sequence of SEQ ID NO: 16; and (ii) a VL comprising an amino acid sequence having at least 90%, at least 95%, at least 98%, at least 99%, or 100% identify to the amino acid sequence of SEQ ID NO: 17.

In another embodiment, in any one of the methods above, the second monoclonal antibody is mAb1 which comprises (i) a VH comprising the amino acid sequence of SEQ ID NO: 16; and (ii) a VL comprising the amino acid sequence of SEQ ID NO: 17.

In another embodiment, in any one of the methods above, the second monoclonal antibody comprises (i) a heavy chain (HC) comprising an amino acid sequence having at least 90%, at least 95%, at least 98%, at least 99%, or 100% identify to the amino acid sequence of SEQ ID NO: 18; and (ii) a light chain (LC) comprising an amino acid sequence having at least 90%, at least 95%, at least 98%, at least 99%, or 100% identify to the amino acid sequence of SEQ ID NO: 19.

In another embodiment, in any one of the methods above, the second monoclonal antibody is mAb1 which comprises (i) a heavy chain (HC) comprising the amino acid sequence of SEQ ID NO: 18; and (ii) a light chain (LC) comprising the amino acid sequence of SEQ ID NO: 19.

In another embodiment, in any one of the methods above, the second monoclonal antibody is mAb1 which comprises (i) a heavy chain (HC) comprising the amino acid sequence of SEQ ID NO: 21; and (ii) a light chain (LC) comprising the amino acid sequence of SEQ ID NO: 19.

In another embodiment, in any one of the methods above, the second monoclonal antibody is mAb1 which comprises (i) two identical heavy chains, each of which comprising the amino acid sequence of SEQ ID NO: 18; and (ii) two identical light chains, each of which comprising the amino acid sequence of SEQ ID NO: 19.

In another embodiment, in any one of the methods above, the second monoclonal antibody is mAb1 which comprises (i) two identical heavy chains, each of which comprising the amino acid sequence of SEQ ID NO: 21; and (ii) two identical light chains, each of which comprising the amino acid sequence of SEQ ID NO: 19.

In another embodiment, in any one of the methods above, the second monoclonal antibody is mAb1 which comprises (i) one heavy chain comprising the amino acid sequence of SEQ ID NO: 18; (ii) one heavy chain comprising the amino acid sequence of SEQ ID NO: 21; and (iii) two identical light chains, each of which comprising the amino acid sequence of SEQ ID NO: 19.

In another embodiment, in any one of the methods above, the first monoclonal antibody is administered via intravenous infusion or via subcutaneous injection (SC).

In another embodiment, in any one of the methods above, the second monoclonal antibody is administered via intravenous infusion or via subcutaneous injection (SC).

In another embodiment, in any one of the methods above, the first monoclonal antibody is administered via intravenous infusion or via subcutaneous injection (SC), and the second monoclonal antibody is administered via intravenous infusion or via subcutaneous injection (SC).

In another embodiment, in any one of the methods above, the first monoclonal antibody is administered via intravenous infusion or via subcutaneous injection (SC), and the second monoclonal antibody is administered via intravenous infusion.

In another embodiment, in any one of the methods above, the first monoclonal antibody is administered at about 10 mg.

In another embodiment, in any one of the methods above, the second monoclonal antibody is administered at about 800 mg.

In another embodiment, in any one of the methods above, the second monoclonal antibody is administered at about 1600 mg.

In another embodiment, in any one of the methods above, the first monoclonal antibody and the second monoclonal antibody are administered at the same time.

In another embodiment, in any one of the methods above, the first monoclonal antibody and the second monoclonal antibody are administered sequentially.

In another embodiment, in any one of the methods above, the first monoclonal antibody is administered every 2 weeks for a duration of 6 weeks.

In another embodiment, in any one of the methods above, the second monoclonal antibody is administered every 4 weeks for a duration of 8 weeks.

In another embodiment, in any one of the methods above, the first monoclonal antibody is administered at about 10 mg every 2 weeks for a duration of 6 weeks.

In another embodiment, in any one of the methods above, the first monoclonal antibody is administered at about 20 mg every 2 weeks for a duration of 6 weeks.

In another embodiment, in any one of the methods above, the second monoclonal antibody is administered at about 800 mg every 4 weeks for a duration of 8 weeks.

In another embodiment, in any one of the methods above, the second monoclonal antibody is administered at about 1600 mg every 4 weeks for a duration of 8 weeks.

In another embodiment, in any one of the methods above, the first monoclonal antibody is administered at about 10 mg every 2 weeks for a duration of 6 weeks and the second monoclonal antibody is administered at about 800 mg every 4 weeks for a duration of 8 weeks.

In another embodiment, in any one of the methods above, the first monoclonal antibody is administered at about 20 mg every 2 weeks for a duration of 6 weeks and the second monoclonal antibody is administered at about 800 mg every 4 weeks for a duration of 8 weeks.

In another embodiment, in any one of the methods above, the first monoclonal antibody is administered at about 10 mg every 2 weeks for a duration of 6 weeks and the second monoclonal antibody is administered at about 1600 mg every 4 weeks for a duration of 8 weeks.

In another embodiment, in any one of the methods above, the first monoclonal antibody is administered at about 20 mg every 2 weeks for a duration of 6 weeks and the second monoclonal antibody is administered at about 1600 mg every 4 weeks for a duration of 8 weeks.

In another embodiment, in any one of the methods above, the first monoclonal antibody is administered at about 10 mg on Week 0 (Day 1), Week 2 (Day 15), Week 4 (Day 29), and Week 6 (Day 43) and the second monoclonal antibody is administered at about 800 mg on Week 0 (Day 1), Week 4 (Day 29), and Week 8 (Day 57).

In another embodiment, in any one of the methods above, the first monoclonal antibody is administered at about 20 mg on Week 0 (Day 1), Week 2 (Day 15), Week 4 (Day 29), and Week 6 (Day 43) and the second monoclonal antibody is administered at about 800 mg on Week 0 (Day 1), Week 4 (Day 29), and Week 8 (Day 57).

In another embodiment, in any one of the methods above, the first monoclonal antibody is administered at about 10 mg on Week 0 (Day 1), Week 2 (Day 15), Week 4 (Day 29), and Week 6 (Day 43) and the second monoclonal antibody is administered at about 1600 mg on Week 0 (Day 1), Week 4 (Day 29), and Week 8 (Day 57).

In another embodiment, in any one of the methods above, the first monoclonal antibody is administered at about 20 mg on Week 0 (Day 1), Week 2 (Day 15), Week 4 (Day 29), and Week 6 (Day 43) and the second monoclonal antibody is administered at about 1600 mg on Week 0 (Day 1), Week 4 (Day 29), and Week 8 (Day 57).

In another embodiment, in any one of the methods above, the first monoclonal antibody is administered via IV infusion at about 10 mg on Week 0 (Day 1), Week 2 (Day 15), Week 4 (Day 29), and Week 6 (Day 43) and the second monoclonal antibody is administered via IV infusion at about 800 mg on Week 0 (Day 1), Week 4 (Day 29), and Week 8 (Day 57).

In another embodiment, in any one of the methods above, the first monoclonal antibody is administered via SC injection at about 20 mg on Week 0 (Day 1), Week 2 (Day 15), Week 4 (Day 29), and Week 6 (Day 43) and the second monoclonal antibody is administered via IV infusion at about 800 mg on Week 0 (Day 1), Week 4 (Day 29), and Week 8 (Day 57).

In another embodiment, in any one of the methods above, the first monoclonal antibody is administered via IV infusion at about 10 mg on Week 0 (Day 1), Week 2 (Day 15), Week 4 (Day 29), and Week 6 (Day 43) and the second monoclonal antibody is administered via IV infusion at about 1600 mg on Week 0 (Day 1), Week 4 (Day 29), and Week 8 (Day 57).

In another embodiment, in any one of the methods above, the first monoclonal antibody is administered via SC injection at about 20 mg on Week 0 (Day 1), Week 2 (Day 15), Week 4 (Day 29), and Week 6 (Day 43) and the second monoclonal antibody is administered via IV infusion at about 1600 mg on Week 0 (Day 1), Week 4 (Day 29), and Week 8 (Day 57).

In another embodiment, in any one of the methods above, after about 25 weeks the patient achieves an HIV viral load of less than about 5,000 copies/mL.

In another embodiment, in any one of the methods above, the HIV-1 viral load of less than about 5,000 copies/mL is achieved without antiretroviral treatment.

In another embodiment, in any one of the methods above, the first monoclonal antibody is administered once every 2 weeks for about 6 weeks.

In another embodiment, in any one of the methods above, after an initial dose, the first monoclonal antibody is administered once every 2 weeks for about 6 weeks.

In another embodiment, in any one of the methods above, the second monoclonal antibody is administered once every 4 weeks for about 8 weeks.

In another embodiment, in any one of the methods above, after an initial dose, the second monoclonal antibody is administered once every 4 weeks for about 8 weeks.

In another embodiment, in any one of the methods above, a HIV-1 viral load of less than about 1000 copies/mL is achieved without antiretroviral treatment at week 24.

In another embodiment, in any one of the methods above, the HIV-1 viral load of less than 1,000 copies/mL is achieved without antiretroviral treatment.

In another embodiment, in any one of the methods above, the HIV-1 viral load of less than 200 copies/mL is achieved without antiretroviral treatment.

In one embodiment, in any one of the methods above, the patient achieves an HIV viral load of less than about 1,000 copies/mL for at least one year.

In one embodiment, in any one of the methods above, the patient achieves an HIV viral load of less than about 200 copies/mL for at least one year.

In another embodiment, in any one of the methods above, the time to viral rebound following interruption of antiretroviral treatment is increased compared with treatment prior to the administration of both the first (i.e., anti-PD-1) and the second (i.e., anti-α4β7) antibodies, wherein the viral rebound is indicated by a viral load greater than about 1000 copies/mL.

In another embodiment, in any one of the methods above, the peak viral load at viral rebound before starting antiretroviral treatment is decreased compared with treatment prior to the administration of both the first (i.e., anti-PD-1) and the second (i.e., anti-α4β7) antibodies, wherein the viral rebound is indicated by a viral load greater than about 1000 copies/mL.

In another embodiment, in any one of the methods above, the patient is an adult patient.

In another embodiment, in any one of the methods above, the patient is living with HIV (PLWH).

In another embodiment, in any one of the methods above, the patient is living with HIV (PLWH) and is virologically suppressed on anti-retroviral therapy (ART) undergoing analytical treatment interruption.

Further benefits of the present disclosure will be apparent to one skilled in the art from reading this patent application. The embodiments of the disclosure described in the following paragraphs are intended to illustrate the invention and should not be deemed to narrow the scope of the invention.

### BRIEF DESCRIPTION OF THE DRAWINGS

FIG. 1A. Exposure-safety relationships for Immune-Related Adverse Events (IRAEs) with anti-PD-1 antibody (mAb2) in study A

FIG. 1B. Pharmacokinetics (PK) of anti-PD-1 antibody (mAb2) in study A showing Mean+SD Serum Concentration-Time Profiles (Linear and Log-Linear Scales)

FIG. 1C. Dose-Normalized Cₘₐₓ Values (Arithmetic Mean±SD) Versus Dose Following Single Doses of anti-PD-1 antibody (mAb2) 10 mg SC and 20 mg SC in Study A

FIG. 1D. Dose-Normalized AUC Values (Arithmetic Mean±SD) Versus Dose Following Single Doses of anti-PD-1 antibody (mAb2) 10 mg SC and 20 mg SC in Study A

FIG. 1E. Mean+SD anti-PD-1 antibody (mAb2) Serum Concentration-Time Profiles Stratified by Treatment-Emergent ADA in Study A (Linear and Log-Linear Scales)

FIG. 1F. Mean+SD % Free PD-1+(CD3+/CD8+) T Cells Versus Time Profiles Following mAb2 Administration in Study A

FIG. 2A. Exposure-safety relationships for Immune-Related Adverse Events (IRAEs) with anti-PD-1 antibody (mAb2) in study B Stage I

FIG. 2B. Exposure-safety relationships for immune-related adverse events (IRAEs) with anti-PD-1 antibody (mAb2) in study B Stage II

FIG. 2C. Pharmacokinetics (PK) of anti-PD-1 antibody (mAb2) in study B Stage I showing Mean+SD Serum Concentration-Time Profiles (Linear and Log-Linear Scales)

FIG. 2D. Pharmacokinetics (PK) of anti-PD-1 antibody mAb2 in study B Stage II showing Mean+SD Serum Concentration-Time Profiles (Linear and Log-Linear Scales)

FIG. 2E. Mean+SD % Free PD-1+(CD3+/CD8+) T Cells Versus Time Profiles Following mAb2 Administration in Study B

FIG. 2F. Dose-Normalized Cₘₐₓ and AUC Values (Arithmetic Mean±SD) Versus Dose Following Dose 1 and Dose 2 of 2 mg and 10 mg of anti-PD-1 antibody (mAb2) in Study B Stage I

FIG. 2G. Mean+SD anti-PD-1 antibody (mAb2) Serum Concentration-Time Profiles Stratified by Treatment-Emergent ADA in Study B Stage I (Linear and Log-Linear Scales)

FIG. 2H. Mean+SD anti-PD-1 antibody (mAb2) Serum Concentration-Time Profiles Stratified by Treatment-Emergent ADA in Study B Stage II (Linear and Log-Linear Scales)

FIG. 2I. HIV RNA Curves in Log Scale for Each Subject for 10 mg of anti-PD-1 antibody mAb2 for Study B Stage II during ATI Period

FIG. 2J. HIV RNA Curves in Log Scale for Each Subject for Pooled placebo during ATI Period

FIG. 2K. HIV RNA Curves in Log Scale for Each Subject for 10 mg of anti-PD-1 antibody mAb2 for Study B Stage I during ATI Period

FIG. 2L. HIV RNA Curves in Log Scale for Each Subject for 2 mg of anti-PD-1 antibody mAb2 for Study B Stage I during ATI Period

FIG. 2M. Exposure-efficacy relationships for viral load control with anti-PD-1 antibody (mAb2) in Study B Stage II

FIG. 3A. Study Scheme of Study E

FIG. 4A. PD-1 Receptor Saturation on Total CD8+ T Cell In Vitro

FIG. 4B. Simulated Serum Nivolumab Concentrations for PLWH Undergo ATI Following IV Q2W Dosing Stratified by Different IV Dose Levels and Different TMDD Models

FIG. 4C. Simulated Serum Nivolumab Concentrations for PLWH Undergo ATI Following IV Q3W Dosing Stratified by Different IV Dose Levels and Different TMDD Models

FIG. 4D. Simulated Serum Nivolumab Concentrations for PLWH Undergo ATI Following IV Q4W Dosing Stratified by Different IV Dose Levels and Different TMDD Models

FIG. 4E. Simulated Serum Pembrolizumab Concentrations for PLWH Undergo ATI Following IV Q2W Dosing Stratified by Different IV Dose Levels and Different TMDD Models

FIG. 4F. Simulated Serum Pembrolizumab Concentrations for PLWH Undergo ATI Following IV Q3W Dosing Stratified by Different IV Dose Levels and Different TMDD Models

FIG. 4G. Simulated Serum Pembrolizumab Concentrations for PLWH Undergo ATI Following IV Q4W Dosing Stratified by Different IV Dose Levels and Different TMDD Models

FIG. 4H. Simulated Serum Cemiplimab Concentrations for PLWH Undergo ATI Following IV Q2W Dosing Stratified by Different IV Dose Levels and Different TMDD Models

FIG. 4I. Simulated Serum Cemiplimab Concentrations for PLWH Undergo ATI Following IV Q3W Dosing Stratified by Different IV Dose Levels and Different TMDD Models

FIG. 4J. Simulated Serum Cemiplimab Concentrations for PLWH Undergo ATI Following IV Q4W Dosing Stratified by Different IV Dose Levels and Different TMDD Models

FIG. 4K. Simulated Serum Nivolumab Concentrations for PLWH Undergo ATI Following SC Q2W Dosing Stratified by Different SC Dose Levels and Different TMDD Models

FIG. 4L. Simulated Serum Nivolumab Concentrations for PLWH Undergo ATI Following SC Q3W Dosing Stratified by Different SC Dose Levels and Different TMDD Models

FIG. 4M. Simulated Serum Nivolumab Concentrations for PLWH Undergo ATI Following SC Q4W Dosing Stratified by Different SC Dose Levels and Different TMDD Models

FIG. 4N. Simulated Serum Pembrolizumab Concentrations for PLWH Undergo ATI Following SC Q2W Dosing Stratified by Different SC Dose Levels and Different TMDD Models

FIG. 4O. Simulated Serum Pembrolizumab Concentrations for PLWH Undergo ATI Following SC Q3W Dosing Stratified by Different SC Dose Levels and Different TMDD Models

FIG. 4P. Simulated Serum Pembrolizumab Concentrations for PLWH Undergo ATI Following SC Q4W Dosing Stratified by Different SC Dose Levels and Different TMDD Models

FIG. 4Q. Simulated Serum Cemiplimab Concentrations for PLWH Undergo ATI Following SC Q2W Dosing Stratified by Different SC Dose Levels and Different TMDD Models

FIG. 4R. Simulated Serum Cemiplimab Concentrations for PLWH Undergo ATI Following SC Q3W Dosing Stratified by Different SC Dose Levels and Different TMDD Models

FIG. 4S. Simulated Serum Cemiplimab Concentrations for PLWH Undergo ATI Following SC Q4W Dosing Stratified by Different SC Dose Levels and Different TMDD Models

### DETAILED DESCRIPTION OF THE INVENTION

The present disclosure relates to methods for treating HIV infection in a human subject or subjects in a population of people living with HIV-1, and in some aspects, in people living with HIV who are responsive to anti-retroviral therapy.

The present disclosure relates to methods for treating HIV infection in a human subject or subjects in a population of people living with HIV-1, and in some aspects, more specifically those on ART, and in some aspects, even more specifically, pausing ART therapy to engage in antibody therapy.

In some embodiments, the present invention provides for the use of a monoclonal antibody which binds to human programmed death receptor 1 (PD-1) for treating HIV infection in a human subject or subjects in a population of people living with HIV-1, and in some embodiments, in people living with HIV who are responsive to anti-retroviral therapy.

In one aspect, the present invention provides for the use of a monoclonal antibody which binds to PD-1 for treating HIV infection in a human subject or subjects in a population of people living with HIV-1, and in some embodiments, in people living with HIV who are responsive to anti-retroviral therapy, by administering about 10 mg to about 20 mg (e.g., about 10 mg or about 20 mg) of the monoclonal antibody.

In one aspect, the present invention provides for the use of a monoclonal antibody which binds to PD-1 for treating HIV infection in a human subject or subjects in a population of people living with HIV-1, and in some embodiments, in people living with HIV who are responsive to anti-retroviral therapy, by administering an initial dose followed by about 10 mg to about 20 mg (e.g., about 10 mg or about 20 mg) of the monoclonal antibody for about 6 weeks.

In one aspect, the present invention provides for the use of a monoclonal antibody which binds to PD-1 for treating HIV infection in a human subject or subjects in a population of people living with HIV-1, and in some embodiments, in people living with HIV who are responsive to anti-retroviral therapy, by administering about 10 mg to about 20 mg (e.g., about 10 mg or about 20 mg) of the monoclonal antibody for about 6 weeks.

In one aspect, the present invention provides for the use of a monoclonal antibody which binds to PD-1 for treating HIV infection in a human subject or subjects in a population of people living with HIV-1, and in some embodiments, in people living with HIV who are responsive to anti-retroviral therapy and undergoing ATI, by administering an initial dose followed by about 10 mg to about 20 mg (e.g., about 10 mg or about 20 mg) of the monoclonal antibody every 2 weeks for about 6 weeks.

In one aspect, the present invention provides for the use of a monoclonal antibody which binds to PD-1 for treating HIV infection in a human subject or subjects in a population of people living with HIV-1, and in some embodiments, in people living with HIV who are responsive to anti-retroviral therapy and undergoing ATI, by administering about 10 mg to about 20 mg (e.g., about 10 mg or about 20 mg) of the monoclonal antibody every 2 weeks for about 6 weeks.

In one aspect, the present invention provides for the use of a monoclonal antibody which binds to PD-1 for treating HIV infection in a human subject or subjects in a population of people living with HIV-1, and in some embodiments, in people living with HIV who are responsive to anti-retroviral therapy and undergoing ATI, by administering about 10 mg to about 20 mg (e.g., about 10 mg or about 20 mg) of the monoclonal antibody on Week 0 (Day 1), Week 2 (Day 15), Week 4 (Day 29), and Week 6 (Day 43).

In one aspect, the present invention provides a method of treating HIV infection in a human subject (e.g., a patient infected with HIV-1) that is living with HIV-1, and in some embodiments, in a human subject (e.g., a patient infected with HIV-1) that is living with HIV who is responsive to anti-retroviral therapy and undergoing ATI, the method comprising administering about 10 mg to about 20 mg (e.g., about 10 mg or about 20 mg) of a monoclonal antibody which binds to human PD-1 on Week 0 (Day 1), Week 2 (Day 15), Week 4 (Day 29), and Week 6 (Day 43).

In one aspect, the present invention provides a method of treating HIV infection in a human subject (e.g., a patient infected with HIV-1) that is living with HIV-1, and in some embodiments, in a human subject (e.g., a patient infected with HIV-1) that is living with HIV who is responsive to anti-retroviral therapy and undergoing ATI, the method comprising administering intravenously about 10 mg of a monoclonal antibody which binds to human PD-1 on Week 0 (Day 1), Week 2 (Day 15), Week 4 (Day 29), and Week 6 (Day 43).

In one aspect, the present invention provides a method of treating HIV infection in a human subject (e.g., a patient infected with HIV-1) that is living with HIV-1, and in some embodiments, in a human subject (e.g., a patient infected with HIV-1) that is living with HIV who is responsive to anti-retroviral therapy and undergoing ATI, the method comprising administering subcutaneously about 20 mg of a monoclonal antibody which binds to human PD-1 on Week 0 (Day 1), Week 2 (Day 15), Week 4 (Day 29), and Week 6 (Day 43).

In one aspect, the present invention provides for the use of a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI.

In one aspect, the present invention provides for the use of an effective amount of a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI, by administering about 800 mg to about 1600 mg (e.g., about 800 mg or about 1600 mg) of the monoclonal antibody.

In one aspect, the present invention provides for the use of an effective amount of a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI, by administering about 800 mg of the monoclonal antibody.

In one aspect, the present invention provides for the use of an effective amount of a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI, by administering about 1600 mg of the monoclonal antibody.

In one aspect, the present invention provides for the use of an effective amount of a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI, by administering about 800 mg to about 1600 mg (e.g., about 800 mg or about 1600 mg) of the monoclonal antibody for about 8 weeks.

In one aspect, the present invention provides for the use of an effective amount of a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI, by administering about 800 mg of the monoclonal antibody for about 8 weeks.

In one aspect, the present invention provides for the use of an effective amount of a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI, by administering about 1600 mg of the monoclonal antibody for about 8 weeks.

In one aspect, the present invention provides for the use of an effective amount of a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI, by administering about 800 mg to about 1600 mg (e.g., about 800 mg or about 1600 mg) of the monoclonal antibody every 4 weeks for about 8 weeks.

In one aspect, the present invention provides for the use of an effective amount of a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI, by administering about 800 mg of the monoclonal antibody every 4 weeks for about 8 weeks.

In one aspect, the present invention provides for the use of an effective amount of a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI, by administering about 1600 mg of the monoclonal antibody every 4 weeks for about 8 weeks.

In one aspect, the present invention provides for the use of a monoclonal antibody which binds to human α4β7 for treating HIV infection in a human subject or subjects in a population of people living with HIV-1, and in some embodiments, in people living with HIV who are responsive to anti-retroviral therapy and undergoing ATI, by administering about 800 mg to about 1600 mg (e.g., about 800 mg or about 1600 mg) of the monoclonal antibody on Week 0 (Day 1), Week 4 (Day 29), and Week 8 (Day 57).

In one aspect, the present invention provides a method of treating HIV infection in a human subject (e.g., a patient infected with HIV-1) that is living with HIV-1, and in some embodiments, in a human subject (e.g., a patient infected with HIV-1) that is living with HIV who is responsive to anti-retroviral therapy and undergoing ATI, the method comprising administering about 800 mg to about 1600 mg (e.g., about 800 mg or about 1600 mg) of a monoclonal antibody which binds to human α4β7 on Week 0 (Day 1), Week 4 (Day 29), and Week 8 (Day 57).

In one aspect, the present invention provides a method of treating HIV infection in a human subject (e.g., a patient infected with HIV-1) that is living with HIV-1, and in some embodiments, in a human subject (e.g., a patient infected with HIV-1) that is living with HIV who is responsive to anti-retroviral therapy and undergoing ATI, the method comprising administering intravenously about 800 mg to about 1600 mg (e.g., about 800 mg or about 1600 mg) of a monoclonal antibody which binds to human α4β7 on Week 0 (Day 1), Week 4 (Day 29), and Week 8 (Day 57).

In one aspect, the present invention provides for the use of an effective amount of a monoclonal antibody which binds to human programmed death receptor 1 (PD-1) and a monoclonal antibody which binds to which binds to human α4β7 for treating HIV infection in a human subject or subjects in a population of people living with HIV-1, and in some embodiments, in people living with HIV who are responsive to anti-retroviral therapy and undergoing ATI.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein after an initial dose the monoclonal antibody which binds to human PD-1 monoclonal antibody is administered at about 10 mg to about 20 mg (e.g., 10 mg or 20 mg) every 2 weeks for a duration of 6 weeks and after an initial dose the monoclonal antibody which binds to human α4β7 is administered at about 800 mg to about 1600 mg (e.g., about 800 mg or about 1600 mg) every 4 weeks for a duration of 8 weeks.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg to about 20 mg (e.g., 10 mg or 20 mg) every 2 weeks for a duration of 6 weeks and the monoclonal antibody which binds to human α4β7 is administered at about 800 mg to about 1600 mg (e.g., about 800 mg or about 1600 mg) every 4 weeks for a duration of 8 weeks.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein after an initial dose the monoclonal antibody which binds to human PD-1 monoclonal antibody is administered at about 10 mg every 2 weeks for a duration of 6 weeks and after an initial dose the monoclonal antibody which binds to human α4β7 is administered at about 800 mg every 4 weeks for a duration of 8 weeks.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg every 2 weeks for a duration of 6 weeks and the monoclonal antibody which binds to human α4β7 is administered at about 800 mg every 4 weeks for a duration of 8 weeks.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg on Week 0 (Day 1), Week 2 (Day 15), Week 4 (Day 29), and Week 6 (Day 43) and the monoclonal antibody which binds to human α4β7 is administered at about 800 mg on Week 0 (Day 1), Week 4 (Day 29), and Week 8 (Day 57).

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg on Week 0 (Day 1), Week 2 (Day 15), Week 4 (Day 29), and Week 6 (Day 43) and the monoclonal antibody which binds to human α4β7 is administered at about 1600 mg on Week 0 (Day 1), Week 4 (Day 29), and Week 8 (Day 57).

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered intravenously at about 10 mg on Week 0 (Day 1), Week 2 (Day 15), Week 4 (Day 29), and Week 6 (Day 43) and the monoclonal antibody which binds to human α4β7 is administered intravenously at about 800 mg on Week 0 (Day 1), Week 4 (Day 29), and Week 8 (Day 57).

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered intravenously at about 10 mg on Week 0 (Day 1), Week 2 (Day 15), Week 4 (Day 29), and Week 6 (Day 43) and the monoclonal antibody which binds to human α4β7 is administered intravenously at about 1600 mg on Week 0 (Day 1), Week 4 (Day 29), and Week 8 (Day 57).

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg on Week 0 (Day 1), Week 2 (Day 15), Week 4 (Day 29), and Week 6 (Day 43) and the monoclonal antibody which binds to human α4β7 is administered at about 800 mg on Week 0 (Day 1), Week 4 (Day 29), and Week 8 (Day 57).

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg on Week 0 (Day 1), Week 2 (Day 15), Week 4 (Day 29), and Week 6 (Day 43) and the monoclonal antibody which binds to human α4β7 is administered at about 1600 mg on Week 0 (Day 1), Week 4 (Day 29), and Week 8 (Day 57).

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered subcutaneously at about 20 mg on Week 0 (Day 1), Week 2 (Day 15), Week 4 (Day 29), and Week 6 (Day 43) and the monoclonal antibody which binds to human α4β7 is administered intravenously at about 800 mg on Week 0 (Day 1), Week 4 (Day 29), and Week 8 (Day 57).

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered subcutaneously at about 20 mg on Week 0 (Day 1), Week 2 (Day 15), Week 4 (Day 29), and Week 6 (Day 43) and the monoclonal antibody which binds to human α4β7 is administered intravenously at about 1600 mg on Week 0 (Day 1), Week 4 (Day 29), and Week 8 (Day 57).

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg to about 20 mg (e.g., 10 mg or 20 mg) every 2 weeks for a duration of 6 weeks and the monoclonal antibody which binds to human α4β7 is administered at about 800 mg to about 1600 mg (e.g., about 800 mg or about 1600 mg) every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg every 2 weeks for a duration of 6 weeks and the monoclonal antibody which binds to human α4β7 is administered at about 800 mg every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg every 2 weeks for a duration of 6 weeks and the monoclonal antibody which binds to human α4β7 is administered at about 1600 mg every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg every 2 weeks for a duration of 6 weeks and the monoclonal antibody which binds to human α4β7 is administered at about 800 mg every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg every 2 weeks for a duration of 6 weeks and the monoclonal antibody which binds to human α4β7 is administered at about 1600 mg every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein after an initial dose the monoclonal antibody which binds to human PD-1 monoclonal antibody is administered at about 10 mg every 2 weeks for a duration of 6 weeks and after an initial dose the monoclonal antibody which binds to human α4β7 is administered at about 800 mg every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg on Week 0 (Day 1), Week 2 (Day 15), Week 4 (Day 29), and Week 6 (Day 43) and the monoclonal antibody which binds to human α4β7 is administered at about 800 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg on Week 0 (Day 1), Week 2 (Day 15), Week 4 (Day 29), and Week 6 (Day 43) and the monoclonal antibody which binds to human α4β7 is administered at about 1600 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered intravenously at about 10 mg on Week 0 (Day 1), Week 2 (Day 15), Week 4 (Day 29), and Week 6 (Day 43) and the monoclonal antibody which binds to human α4β7 is administered intravenously at about 800 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered intravenously at about 10 mg on Week 0 (Day 1), Week 2 (Day 15), Week 4 (Day 29), and Week 6 (Day 43) and the monoclonal antibody which binds to human α4β7 is administered intravenously at about 1600 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg on Week 0 (Day 1), Week 2 (Day 15), Week 4 (Day 29), and Week 6 (Day 43) and the monoclonal antibody which binds to human α4β7 is administered at about 800 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg on Week 0 (Day 1), Week 2 (Day 15), Week 4 (Day 29), and Week 6 (Day 43) and the monoclonal antibody which binds to human α4β7 is administered at about 1600 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered subcutaneously at about 20 mg on Week 0 (Day 1), Week 2 (Day 15), Week 4 (Day 29), and Week 6 (Day 43) and the monoclonal antibody which binds to human α4β7 is administered intravenously at about 800 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered subcutaneously at about 20 mg on Week 0 (Day 1), Week 2 (Day 15), Week 4 (Day 29), and Week 6 (Day 43) and the monoclonal antibody which binds to human α4β7 is administered intravenously at about 1600 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein after an initial dose the monoclonal antibody which binds to human PD-1 monoclonal antibody is administered at about 10 mg to about 20 mg (e.g., 10 mg or 20 mg) every 2 weeks for a duration of 6 weeks and after an initial dose the monoclonal antibody which binds to human α4β7 is administered at about 800 mg to about 1600 mg (e.g., about 800 mg or about 1600 mg) every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg to about 20 mg (e.g., 10 mg or 20 mg) every 2 weeks for a duration of 6 weeks and the monoclonal antibody which binds to human α4β7 is administered at about 800 mg to about 1600 mg (e.g., about 800 mg or about 1600 mg) every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg every 2 weeks for a duration of 6 weeks and the monoclonal antibody which binds to human α4β7 is administered at about 800 mg every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg every 2 weeks for a duration of 6 weeks and the monoclonal antibody which binds to human α4β7 is administered at about 1600 mg every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg every 2 weeks for a duration of 6 weeks and the monoclonal antibody which binds to human α4β7 is administered at about 800 mg every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg every 2 weeks for a duration of 6 weeks and the monoclonal antibody which binds to human α4β7 is administered at about 1600 mg every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg on Week 0 (Day 1), Week 2 (Day 15), Week 4 (Day 29), and Week 6 (Day 43) and the monoclonal antibody which binds to human α4β7 is administered at about 800 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg on Week 0 (Day 1), Week 2 (Day 15), Week 4 (Day 29), and Week 6 (Day 43) and the monoclonal antibody which binds to human α4β7 is administered at about 1600 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered intravenously at about 10 mg on Week 0 (Day 1), Week 2 (Day 15), Week 4 (Day 29), and Week 6 (Day 43) and the monoclonal antibody which binds to human α4β7 is administered intravenously at about 800 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered intravenously at about 10 mg on Week 0 (Day 1), Week 2 (Day 15), Week 4 (Day 29), and Week 6 (Day 43) and the monoclonal antibody which binds to human α4β7 is administered intravenously at about 1600 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg on Week 0 (Day 1), Week 2 (Day 15), Week 4 (Day 29), and Week 6 (Day 43) and the monoclonal antibody which binds to human α4β7 is administered at about 800 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg on Week 0 (Day 1), Week 2 (Day 15), Week 4 (Day 29), and Week 6 (Day 43) and the monoclonal antibody which binds to human α4β7 is administered at about 1600 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered subcutaneously at about 20 mg on Week 0 (Day 1), Week 2 (Day 15), Week 4 (Day 29), and Week 6 (Day 43) and the monoclonal antibody which binds to human α4β7 is administered intravenously at about 800 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered subcutaneously at about 20 mg on Week 0 (Day 1), Week 2 (Day 15), Week 4 (Day 29), and Week 6 (Day 43) and the monoclonal antibody which binds to human α4β7 is administered intravenously at about 1600 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein after an initial dose the monoclonal antibody which binds to human PD-1 monoclonal antibody is administered at about 10 mg to about 20 mg (e.g., 10 mg or 20 mg) every 2 weeks for a duration of 6 weeks and after an initial dose the monoclonal antibody which binds to human α4β7 is administered at about 800 mg to about 1600 mg (e.g., about 800 mg or about 1600 mg) every 4 weeks for a duration of 8 weeks to achieve a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg to about 20 mg (e.g., 10 mg or 20 mg) every 2 weeks for a duration of 6 weeks and the monoclonal antibody which binds to human α4β7 is administered at about 800 mg to about 1600 mg (e.g., about 800 mg or about 1600 mg) every 4 weeks for a duration of 8 weeks to achieve a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg every 2 weeks for a duration of 6 weeks and the monoclonal antibody which binds to human α4β7 is administered at about 800 mg every 4 weeks for a duration of 8 weeks to achieve a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg every 2 weeks for a duration of 6 weeks and the monoclonal antibody which binds to human α4β7 is administered at about 1600 mg every 4 weeks for a duration of 8 weeks to achieve a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg on Week 0 (Day 1), Week 2 (Day 15), Week 4 (Day 29), and Week 6 (Day 43) and the monoclonal antibody which binds to human α4β7 is administered at about 800 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 10 mg on Week 0 (Day 1), Week 2 (Day 15), Week 4 (Day 29), and Week 6 (Day 43) and the monoclonal antibody which binds to human α4β7 is administered at about 1600 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered intravenously at about 10 mg on Week 0 (Day 1), Week 2 (Day 15), Week 4 (Day 29), and Week 6 (Day 43) and the monoclonal antibody which binds to human α4β7 is administered intravenously at about 800 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered intravenously at about 10 mg on Week 0 (Day 1), Week 2 (Day 15), Week 4 (Day 29), and Week 6 (Day 43) and the monoclonal antibody which binds to human α4β7 is administered intravenously at about 1600 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg on Week 0 (Day 1), Week 2 (Day 15), Week 4 (Day 29), and Week 6 (Day 43) and the monoclonal antibody which binds to human α4β7 is administered at about 800 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered at about 20 mg on Week 0 (Day 1), Week 2 (Day 15), Week 4 (Day 29), and Week 6 (Day 43) and the monoclonal antibody which binds to human α4β7 is administered at about 1600 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered subcutaneously at about 20 mg on Week 0 (Day 1), Week 2 (Day 15), Week 4 (Day 29), and Week 6 (Day 43) and the monoclonal antibody which binds to human α4β7 is administered intravenously at about 800 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of a monoclonal antibody which binds to human programmed death receptor 1 (PD-1) and a monoclonal antibody which binds to human α4β7, wherein the monoclonal antibody which binds to human PD-1 monoclonal antibody is administered subcutaneously at about 20 mg on Week 0 (Day 1), Week 2 (Day 15), Week 4 (Day 29), and Week 6 (Day 43) and the monoclonal antibody which binds to human α4β7 is administered intravenously at about 1600 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides for the use of mAb1 to treat HIV.

In one aspect, a method of the present invention provides for administering an effective amount of mAb1 to treat HIV via intravenous or subcutaneous infusion.

In one aspect, mAb1 is an anti-α4β7 integrin monoclonal antibody (Ab) with preserved fragment crystallizable (Fc) region functionality. Recently, data generated show that mAb1 antibody binds to cell-free HIV in vitro. Thus, mAb1 binding to α4β7 of HIV virions could lead to the formation of immune complexes that can be recognized by the Fc gamma receptors expressed on immune cells, resulting in modulation of host antiviral immune responses. In one aspect, mAb1 is the anti-α4β7 integrin monoclonal antibody described in U.S. Pat. No. 11,639,390, incorporated by reference herein.

In one aspect, antibody mAb1 comprises variable regions and CDRs (complementary determining regions) which can be identified according to rules developed in the art and/or by aligning sequences against a database of known variable regions.

In certain aspects, mAb1 comprises a heavy chain variable region (VH) CDR #1 having the amino acid sequence GFNIKNTYMH (SEQ ID NO:11); a VH CDR #2 having the amino acid sequence RIDPAKGHTEYAPKFLG (SEQ ID NO:12); a VH CDR #3 having the amino acid sequence VDV; a light chain variable region (VL) CDR #1 having the amino acid sequence HASQDISDNIG (SEQ ID NO:13); a VL CDR #2 having the amino acid sequence HGTNLED (SEQ ID NO:14); and a VL CDR #3 having the amino acid sequence VQYAQFPWT (SEQ ID NO:15). In some embodiments, the CDRs described herein are based on the Kabat numbering scheme.

In certain aspects, mAb1 comprises a heavy chain variable region (VH) comprising the amino acid sequence shown as SEQ ID NO: 16; and a light chain variable region (VL) comprising the amino acid sequence shown as SEQ ID NO 17.

In certain aspects, mAb1 comprises a heavy chain comprising the amino acid sequence shown as SEQ ID NO: 18 and may also have a C-terminal lysine truncated heavy chain of SEQ ID NO: 21; and a light chain comprising the amino acid sequence shown as SEQ ID NO: 19.

In certain aspects, mAb1 comprises two identical heavy chains, each comprising the amino acid sequence shown as SEQ ID NO: 18; and two identical light chains, each comprising the amino acid sequence shown as SEQ ID NO: 19.

In certain aspects, mAb1 comprises two identical heavy chains, each comprising the amino acid sequence shown as SEQ ID NO: 21; and two identical light chains, each comprising the amino acid sequence shown as SEQ ID NO: 19.

In certain aspects, mAb1 comprises a first heavy chain comprising the amino acid sequence shown as SEQ ID NO: 18; a second heavy chain comprising the amino acid sequence shown as SEQ ID NO: 21; and two identical light chains, each comprising the amino acid sequence shown as SEQ ID NO: 19.

In some aspects, methods of treating HIV infection described herein comprise administering a therapeutically effective amount of a monoclonal antibody that binds to PD-1 (e.g., human PD-1). In some embodiments, the monoclonal antibody that binds to human PD-1 is mAb2.

In certain embodiments, the anti-PD-1 antibody described herein (e.g., mAb2) is a humanized, recombinant immunoglobulin gamma 1 (IgG1) L234A L235A monoclonal antibody that binds to cell surface expressed PD-1 and blocks the interaction of the receptor with its ligands. Chronic HIV infection results in immune exhaustion and dampening of HIV-specific cellular immune responses by upregulation of inhibitory co-receptors or “immune checkpoints,” such as PD-1. Anti-PD-1 antibodies contribute to the reversal of T cell exhaustion by blocking the interaction of PD-1 with ligands PD-L1 and PD-L2, thereby interfering with the negative immunological signal. Based on clinical data in HIV-infected patients, anti-PD-1 monoclonal antibodies in the context of HIV-1 infection can facilitate viral clearance and control of chronic viral infection by mediating functional restoration of exhausted virus-specific CD8+ T cells; facilitate activation of exhausted latently infected CD4+ T cells potentially leading to latency reversal; and restore CD4+ T cell functionality leading to improved virus-specific CD8+ T cell and B cell responses.

Anti-PD-1 monoclonal antibodies comprise variable regions and CDRs (complementary determining regions) which can be identified according to rules developed in the art and/or by aligning sequences against a database of known variable regions.

Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. For example, CDRs may be identified in accordance with one of the schemes provided by Kabat et al. (1991) Sequences of Proteins of Immunological Interest (5^{th} Ed.), U.S. Dept. of Health and Human Services, PHS, NIH, NIH Publication No. 91-3242 (referred to herein as “Kabat”); or in accordance with AbM (Oxford Molecular/MSI Pharmacopia) (referred to herein as “AbM”). AbM can be obtained from the Abysis database at www.bioinf.org.uk/abs (maintained by A. C. Martin in the Department of Biochemistry & Molecular Biology University College London).

In some embodiments, mAb2 comprises a heavy chain variable region (VH) CDR #1 having the amino acid sequence GYTFTHYGMN (SEQ ID NO:1); a VH CDR #2 having the amino acid sequence WVNTYTGEPTYADDFKG (SEQ ID NO:2); a VH CDR #3 having the amino acid sequence EGEGLGFGD (SEQ ID NO:3); a light chain variable region (VL) CDR #1 having the amino acid sequence RSSQSIVHSHGDTYLE (SEQ ID NO:4); a VL CDR #2 having the amino acid sequence KVSNRFS (SEQ ID NO:5); and a VL CDR #3 having the amino acid sequence FQGSHIPVT (SEQ ID NO:6). In some embodiments, the CDRs described herein are based on the Kabat numbering scheme.

In embodiments, mAb2 comprises a heavy chain variable region (VH) comprising the amino acid sequence shown as SEQ ID NO: 7; and a light chain variable region (VL) comprising the amino acid sequence shown as SEQ ID NO: 8.

In embodiments, mAb2 comprises a heavy chain comprising the amino acid sequence disclosed as SEQ ID NO: 9 and may also have a C-terminal lysine truncated heavy chain of SEQ ID NO: 20; and a light chain comprising the amino acid sequence disclosed as SEQ ID NO: 10.

In certain aspects, mAb2 comprises two identical heavy chains, each comprising the amino acid sequence shown as SEQ ID NO: 9; and two identical light chains, each comprising the amino acid sequence shown as SEQ ID NO: 10.

In certain aspects, mAb2 comprises two identical heavy chains, each comprising the amino acid sequence shown as SEQ ID NO: 20; and two identical light chains, each comprising the amino acid sequence shown as SEQ ID NO: 10.

In certain aspects, mAb2 comprises a first heavy chain comprising the amino acid sequence shown as SEQ ID NO: 9; a second heavy chain comprising the amino acid sequence shown as SEQ ID NO: 20; and two identical light chains, each comprising the amino acid sequence shown as SEQ ID NO: 10.

In one aspect, the anti-PD-1 antibody used in the methods described herein (e.g., mAb2) is a humanized, recombinant immunoglobulin gamma 1 (IgG1) L234A L235A monoclonal antibody described in U.S. Pat. No. 9,914,783, incorporated by reference herein.

In one aspect, the anti-PD-1 antibody used in the methods described herein (e.g., mAb2) is Budigalimab.

In some embodiments, the anti-PD-1 antibody used in the methods described herein is Nivolumab, Pembrolizumab, and Cemiplimab.

In one aspect, the present invention provides for the use of mAb2 for treating HIV infection in a human subject or subjects in a population of people living with HIV-1, and in some embodiments, in people living with HIV who are responsive to anti-retroviral therapy, by administering about 10 mg to about 20 mg (e.g., about 10 mg or about 20 mg) of mAb2.

In one aspect, the present invention provides for the use of an effective amount of mAb2 for treating HIV infection in a human subject or subjects in a population of people living with HIV-1, and in some embodiments, in people living with HIV who are responsive to anti-retroviral therapy, by administering about 10 mg to about 20 mg (e.g., about 10 mg or about 20 mg) of mAb2 for about 6 weeks.

In one aspect, the present invention provides for the use of an effective amount of mAb2 for treating HIV infection in a human subject or subjects in a population of people living with HIV-1, and in some embodiments, in people living with HIV who are responsive to anti-retroviral therapy and undergoing ATI, by administering about 10 mg to about 20 mg (e.g., about 10 mg or about 20 mg) of mAb2 every 2 weeks for about 6 weeks.

In one aspect, the present invention provides for the use of an effective amount of mAb2 for treating HIV infection in a human subject or subjects in a population of people living with HIV-1, and in some embodiments, in people living with HIV who are responsive to anti-retroviral therapy and undergoing ATI, by administering about 10 mg to about 20 mg (e.g., about 10 mg or about 20 mg) of mAb2 on Week 0 (Day 1), Week 2 (Day 15), Week 4 (Day 29), and Week 6 (Day 43).

In one aspect, the present invention provides a method of treating HIV infection in a human subject (e.g., a patient infected with HIV-1) that is living with HIV-1, and in some embodiments, in a human subject (e.g., a patient infected with HIV-1) living with HIV who is responsive to anti-retroviral therapy and undergoing ATI, the method comprising administering about 10 mg to about 20 mg (e.g., about 10 mg or about 20 mg) of mAb2 on Week 0 (Day 1), Week 2 (Day 15), Week 4 (Day 29), and Week 6 (Day 43).

In one aspect, the present invention provides a method of treating HIV infection in a human subject (e.g., a patient infected with HIV-1) that is living with HIV-1, and in some embodiments, in a human subject (e.g., a patient infected with HIV-1) living with HIV who is responsive to anti-retroviral therapy and undergoing ATI, the method comprising administering intravenously about 10 mg of mAb2 on Week 0 (Day 1), Week 2 (Day 15), Week 4 (Day 29), and Week 6 (Day 43).

In one aspect, the present invention provides a method of treating HIV infection in a human subject (e.g., a patient infected with HIV-1) that is living with HIV-1, and in some embodiments, in a human subject (e.g., a patient infected with HIV-1) living with HIV who is responsive to anti-retroviral therapy and undergoing ATI, the method comprising administering subcutaneously about 20 mg of mAb2 on Week 0 (Day 1), Week 2 (Day 15), Week 4 (Day 29), and Week 6 (Day 43).

In one aspect, the present invention provides for the use of an effective amount of mAb1 for treating HIV infection in a human subject or subjects in a population of people living with HIV-1, and in some embodiments, in people living with HIV who are responsive to anti-retroviral therapy and undergoing ATI.

In one aspect, the present invention provides for the use of an effective amount of mAb1 for treating HIV infection in a human subject or subjects in a population of people living with HIV-1, and in some embodiments, in people living with HIV who are responsive to anti-retroviral therapy and undergoing ATI, by administering about 800 mg to about 1600 mg (e.g., about 800 mg or about 1600 mg) of mAb1.

In one aspect, the present invention provides for the use of an effective amount of mAb1 for treating HIV infection in a human subject or subjects in a population of people living with HIV-1, and in some embodiments, in people living with HIV who are responsive to anti-retroviral therapy and undergoing ATI by administering about 800 mg of mAb1.

In one aspect, the present invention provides for the use of an effective amount of mAb1 for treating HIV infection in a human subject or subjects in a population of people living with HIV-1, and in some embodiments, in people living with HIV who are responsive to anti-retroviral therapy and undergoing ATI by administering about 1600 mg of mAb1.

In one aspect, the present invention provides for the use of an effective amount of mAb1 for treating HIV infection in a human subject or subjects in a population of people living with HIV-1, and in some embodiments, in people living with HIV who are responsive to anti-retroviral therapy and undergoing ATI, by administering about 800 mg to about 1600 mg (e.g., about 800 mg or about 1600 mg) of mAb1 for about 8 weeks.

In one aspect, the present invention provides for the use of an effective amount of mAb1 for treating HIV infection in a human subject or subjects in a population of people living with HIV-1, and in some embodiments, in people living with HIV who are responsive to anti-retroviral therapy and undergoing ATI by administering about 800 mg of mAb1 for about 8 weeks.

In one aspect, the present invention provides for the use of an effective amount of mAb1 for treating HIV infection in a human subject or subjects in a population of people living with HIV-1, and in some embodiments, in people living with HIV who are responsive to anti-retroviral therapy and undergoing ATI by administering about 1600 mg of mAb1 for about 8 weeks.

In one aspect, the present invention provides for the use of an effective amount of mAb1 for treating HIV infection in a human subject or subjects in a population of people living with HIV-1, and in some embodiments, in people living with HIV who are responsive to anti-retroviral therapy and undergoing ATI, by administering about 800 mg to about 1600 mg (e.g., about 800 mg or about 1600 mg) of mAb1 every 4 weeks for about 8 weeks.

In one aspect, the present invention provides for the use of an effective amount of mAb1 for treating HIV infection in a human subject or subjects in a population of people living with HIV-1, and in some embodiments, in people living with HIV who are responsive to anti-retroviral therapy and undergoing ATI by administering about 800 mg of mAb1 every 4 weeks for about 8 weeks.

In one aspect, the present invention provides for the use of an effective amount of mAb1 for treating HIV infection in a human subject or subjects in a population of people living with HIV-1, and in some embodiments, in people living with HIV who are responsive to anti-retroviral therapy and undergoing ATI by administering about 1600 mg of mAb1 every 4 weeks for about 8 weeks.

In one aspect, the present invention provides for the use of mAb1 for treating HIV infection in a human subject or subjects in a population of people living with HIV-1, and in some embodiments, in people living with HIV who are responsive to anti-retroviral therapy and undergoing ATI, by administering about 800 mg to about 1600 mg (e.g., about 800 mg or about 1600 mg) of mAb1 on Week 0 (Day 1), Week 4 (Day 29), and Week 8 (Day 57).

In one aspect, the present invention provides a method of treating HIV infection in a human subject (e.g., a patient infected with HIV-1) that is living with HIV-1, and in some embodiments, in a human subject (e.g., a patient infected with HIV-1) that is living with HIV who is responsive to anti-retroviral therapy and undergoing ATI, the method comprising administering about 800 mg to about 1600 mg (e.g., about 800 mg or about 1600 mg) of mAb1 on Week 0 (Day 1), Week 4 (Day 29), and Week 8 (Day 57).

In one aspect, the present invention provides a method of treating HIV infection in a human subject (e.g., a patient infected with HIV-1) that is living with HIV-1, and in some embodiments, in a human subject (e.g., a patient infected with HIV-1) living with HIV who is responsive to anti-retroviral therapy and undergoing ATI, the method comprising administering intravenously about 800 mg to about 1600 mg (e.g., about 800 mg or about 1600 mg) of mAb1 on Week 0 (Day 1), Week 4 (Day 29), and Week 8 (Day 57).

In one aspect, the present invention provides for the use of an effective amount of mAb2 and mAb1 for treating HIV infection in a human subject or subjects in a population of people living with HIV-1, and in some embodiments, in people living with HIV who are responsive to anti-retroviral therapy and undergoing ATI.

In one aspect, the present invention provides a method of treating HIV infection in a patient infected with HIV-1, the method comprising: administering an effective amount of mAb2 and mAb1, wherein after an initial dose mAb2 is administered at about 10 mg to about 20 mg (e.g., 10 mg or 20 mg) every 2 weeks for a duration of 6 weeks and after an initial dose mAb1 is administered at about 800 mg to about 1600 mg (e.g., about 800 mg or about 1600 mg) every 4 weeks for a duration of 8 weeks.

In one aspect, the present invention provides a method of treating HIV infection in a patient infected with HIV-1, the method comprising: administering an effective amount of mAb2 and mAb1, wherein mAb2 is administered at about 10 mg to about 20 mg (e.g., 10 mg or 20 mg) every 2 weeks for a duration of 6 weeks and mAb1 is administered at about 800 mg to about 1600 mg (e.g., about 800 mg or about 1600 mg) every 4 weeks for a duration of 8 weeks.

In one aspect, the present invention provides a method of treating HIV infection in a patient infected with HIV-1, the method comprising: administering an effective amount of mAb2 and mAb1, wherein mAb2 is administered at about 10 mg every 2 weeks for a duration of 6 weeks and mAb1 is administered at about 800 mg every 4 weeks for a duration of 8 weeks.

In one aspect, the present invention provides a method of treating HIV infection in a patient infected with HIV-1, the method comprising: administering an effective amount of mAb2 and mAb1, wherein mAb2 is administered at about 20 mg every 2 weeks for a duration of 6 weeks and mAb1 is administered at about 800 mg every 4 weeks for a duration of 8 weeks.

In one aspect, the present invention provides a method of treating HIV infection in a patient infected with HIV-1, the method comprising: administering an effective amount of mAb2 and mAb1, wherein mAb2 is administered at about 10 mg on Week 0 (Day 1), Week 2 (Day 15), Week 4 (Day 29), and Week 6 (Day 43) and mAb1 is administered at about 800 mg on Week 0 (Day 1), Week 4 (Day 29), and Week 8 (Day 57).

In one aspect, the present invention provides a method of treating HIV infection in a patient infected with HIV-1, the method comprising: administering an effective amount of mAb2 and mAb1, wherein mAb2 is administered at about 10 mg on Week 0 (Day 1), Week 2 (Day 15), Week 4 (Day 29), and Week 6 (Day 43) and mAb1 is administered at about 1600 mg on Week 0 (Day 1), Week 4 (Day 29), and Week 8 (Day 57).

In one aspect, the present invention provides a method of treating HIV infection in a patient infected with HIV-1, the method comprising: administering an effective amount of mAb2 and mAb1, wherein mAb2 is administered intravenously at about 10 mg on Week 0 (Day 1), Week 2 (Day 15), Week 4 (Day 29), and Week 6 (Day 43) and mAb1 is administered intravenously at about 800 mg on Week 0 (Day 1), Week 4 (Day 29), and Week 8 (Day 57).

In one aspect, the present invention provides a method of treating HIV infection in a patient infected with HIV-1, the method comprising: administering an effective amount of mAb2 and mAb1, wherein mAb2 is administered intravenously at about 10 mg on Week 0 (Day 1), Week 2 (Day 15), Week 4 (Day 29), and Week 6 (Day 43) and mAb1 is administered intravenously at about 1600 mg on Week 0 (Day 1), Week 4 (Day 29), and Week 8 (Day 57).

In one aspect, the present invention provides a method of treating HIV infection in a patient infected with HIV-1, the method comprising: administering an effective amount of mAb2 and mAb1, wherein mAb2 is administered at about 20 mg on Week 0 (Day 1), Week 2 (Day 15), Week 4 (Day 29), and Week 6 (Day 43) and mAb1 is administered at about 800 mg on Week 0 (Day 1), Week 4 (Day 29), and Week 8 (Day 57).

In one aspect, the present invention provides a method of treating HIV infection in a patient infected with HIV-1, the method comprising: administering an effective amount of mAb2 and mAb1, wherein mAb2 is administered at about 20 mg on Week 0 (Day 1), Week 2 (Day 15), Week 4 (Day 29), and Week 6 (Day 43) and mAb1 is administered at about 1600 mg on Week 0 (Day 1), Week 4 (Day 29), and Week 8 (Day 57).

In one aspect, the present invention provides a method of treating HIV infection in a patient infected with HIV-1, the method comprising: administering an effective amount of mAb2 and mAb1, wherein mAb2 is administered subcutaneously at about 20 mg on Week 0 (Day 1), Week 2 (Day 15), Week 4 (Day 29), and Week 6 (Day 43) and mAb1 is administered intravenously at about 800 mg on Week 0 (Day 1), Week 4 (Day 29), and Week 8 (Day 57).

In one aspect, the present invention provides a method of treating HIV infection in a patient infected with HIV-1, the method comprising: administering an effective amount of mAb2 and mAb1, wherein mAb2 is administered subcutaneously at about 20 mg on Week 0 (Day 1), Week 2 (Day 15), Week 4 (Day 29), and Week 6 (Day 43) and mAb1 is administered intravenously at about 1600 mg on Week 0 (Day 1), Week 4 (Day 29), and Week 8 (Day 57).

In one aspect, the present invention provides a method of treating HIV infection in a patient infected with HIV-1, the method comprising: administering an effective amount of mAb2 and mAb1, wherein after an initial dose mAb2 is administered at about 10 mg to about 20 mg (e.g., 10 mg or 20 mg) every 2 weeks for a duration of 6 weeks and after an initial dose mAb1 is administered at about 800 mg to about 1600 mg (e.g., about 800 mg or about 1600 mg) every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg to about 20 mg (e.g., 10 mg or 20 mg) every 2 weeks for a duration of 6 weeks and mAb1 is administered at about 800 mg to about 1600 mg (e.g., about 800 mg or about 1600 mg) every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein after an initial dose mAb2 is administered at about 10 mg every 2 weeks for a duration of 6 weeks and after an initial dose mAb1 is administered at about 800 mg every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg every 2 weeks for a duration of 6 weeks and mAb1 is administered at about 800 mg every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg every 2 weeks for a duration of 6 weeks and mAb1 is administered at about 1600 mg every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg every 2 weeks for a duration of 6 weeks and mAb1 is administered at about 800 mg every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg every 2 weeks for a duration of 6 weeks and mAb1 is administered at about 1600 mg every 4 weeks for a duration of 8 weeks to achieve a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg on Week 0 (Day 1), Week 2 (Day 15), Week 4 (Day 29), and Week 6 (Day 43) and mAb1 is administered at about 800 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg on Week 0 (Day 1), Week 2 (Day 15), Week 4 (Day 29), and Week 6 (Day 43) and mAb1 is administered at about 1600 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered intravenously at about 10 mg on Week 0 (Day 1), Week 2 (Day 15), Week 4 (Day 29), and Week 6 (Day 43) and mAb1 is administered intravenously at about 800 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered intravenously at about 10 mg on Week 0 (Day 1), Week 2 (Day 15), Week 4 (Day 29), and Week 6 (Day 43) and mAb1 is administered intravenously at about 1600 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg on Week 0 (Day 1), Week 2 (Day 15), Week 4 (Day 29), and Week 6 (Day 43) and mAb1 is administered at about 800 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg on Week 0 (Day 1), Week 2 (Day 15), Week 4 (Day 29), and Week 6 (Day 43) and mAb1 is administered at about 1600 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered subcutaneously at about 20 mg on Week 0 (Day 1), Week 2 (Day 15), Week 4 (Day 29), and Week 6 (Day 43) and mAb1 is administered intravenously at about 800 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered subcutaneously at about 20 mg on Week 0 (Day 1), Week 2 (Day 15), Week 4 (Day 29), and Week 6 (Day 43) and mAb1 is administered intravenously at about 1600 mg on Week 0 (Day 1), Week 4 (Day 29), and Week 8 (Day 57), and wherein the patient achieves a HIV-1 viral load of less than about 5,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg to about 20 mg (e.g., 10 mg or 20 mg) every 2 weeks for a duration of 6 weeks and mAb1 is administered at about 800 mg to about 1600 mg (e.g., about 800 mg or about 1600 mg) every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg every 2 weeks for a duration of 6 weeks and mAb1 is administered at about 800 mg every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg every 2 weeks for a duration of 6 weeks and mAb1 is administered at about 1600 mg every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg every 2 weeks for a duration of 6 weeks and mAb1 is administered at about 800 mg every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg every 2 weeks for a duration of 6 weeks and mAb1 is administered at about 1600 mg every 4 weeks for a duration of 8 weeks to achieve a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg on Week 0 (Day 1), Week 2 (Day 15), Week 4 (Day 29), and Week 6 (Day 43) and mAb1 is administered at about 800 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg on Week 0 (Day 1), Week 2 (Day 15), Week 4 (Day 29), and Week 6 (Day 43) and mAb1 is administered at about 1600 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg on Week 0 (Day 1), Week 2 (Day 15), Week 4 (Day 29), and Week 6 (Day 43) and mAb1 is administered at about 800 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg on Week 0 (Day 1), Week 2 (Day 15), Week 4 (Day 29), and Week 6 (Day 43) and mAb1 is administered at about 1600 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered intravenously at about 10 mg on Week 0 (Day 1), Week 2 (Day 15), Week 4 (Day 29), and Week 6 (Day 43) and mAb1 is administered intravenously at about 800 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered intravenously at about 10 mg on Week 0 (Day 1), Week 2 (Day 15), Week 4 (Day 29), and Week 6 (Day 43) and mAb1 is administered intravenously at about 1600 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered subcutaneously at about 20 mg on Week 0 (Day 1), Week 2 (Day 15), Week 4 (Day 29), and Week 6 (Day 43) and mAb1 is administered intravenously at about 800 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered subcutaneously at about 20 mg on Week 0 (Day 1), Week 2 (Day 15), Week 4 (Day 29), and Week 6 (Day 43) and mAb1 is administered intravenously at about 1600 mg on Week 0 (Day 1), Week 4 (Day 29), and Week 8 (Day 57), and wherein the patient achieves a HIV-1 viral load of less than about 1,000 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg to about 20 mg (e.g., 10 mg or 20 mg) every 2 weeks for a duration of 6 weeks and mAb1 is administered at about 800 mg to about 1600 mg (e.g., about 800 mg or about 1600 mg) every 4 weeks for a duration of 8 weeks to achieve a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg every 2 weeks for a duration of 6 weeks and mAb1 is administered at about 800 mg every 4 weeks for a duration of 8 weeks to achieve a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg every 2 weeks for a duration of 6 weeks and mAb1 is administered at about 1600 mg every 4 weeks for a duration of 8 weeks to achieve a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg every 2 weeks for a duration of 6 weeks and mAb1 is administered at about 800 mg every 4 weeks for a duration of 8 weeks to achieve a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg every 2 weeks for a duration of 6 weeks and mAb1 is administered at about 1600 mg every 4 weeks for a duration of 8 weeks to achieve a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg on Week 0 (Day 1), Week 2 (Day 15), Week 4 (Day 29), and Week 6 (Day 43) and mAb1 is administered at about 800 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 10 mg on Week 0 (Day 1), Week 2 (Day 15), Week 4 (Day 29), and Week 6 (Day 43) and mAb1 is administered at about 1600 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered intravenously at about 10 mg on Week 0 (Day 1), Week 2 (Day 15), Week 4 (Day 29), and Week 6 (Day 43) and mAb1 is administered intravenously at about 800 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered intravenously at about 10 mg on Week 0 (Day 1), Week 2 (Day 15), Week 4 (Day 29), and Week 6 (Day 43) and mAb1 is administered intravenously at about 1600 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg on Week 0 (Day 1), Week 2 (Day 15), Week 4 (Day 29), and Week 6 (Day 43) and mAb1 is administered at about 800 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered at about 20 mg on Week 0 (Day 1), Week 2 (Day 15), Week 4 (Day 29), and Week 6 (Day 43) and mAb1 is administered at about 1600 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered subcutaneously at about 20 mg on Week 0 (Day 1), Week 2 (Day 15), Week 4 (Day 29), and Week 6 (Day 43) and mAb1 is administered intravenously at about 800 mg on Week 0 (Day 1), Week 4 (Day 29), and Week 8 (Day 57), and wherein the patient achieves a HIV-1 viral load of less than about 200 copies/mL without antiretroviral treatment.

In one aspect, the present invention provides a method of treating HIV infection in a patient infected with HIV-1, the method comprising: administering an effective amount of mAb2 and mAb1, wherein mAb2 is administered subcutaneously at about 20 mg on Week 0 (Day 1), Week 2 (Day 15), Week 4 (Day 29), and Week 6 (Day 43) and mAb1 is administered intravenously at about 1600 mg on Week 0 (Day 1), Week 4 (Day 29), and Week 8 (Day 57), and wherein the patient achieves a HIV-1 viral load of less than about 200 copies/mL without antiretroviral treatment.

### Definitions

As used in the specification and the appended claims, unless specified to the contrary, the following terms have the meaning indicated.

Where a numeric range is recited herein, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated.

The term “about” generally refers to a range of numbers that one of skill in the art would consider equivalent to the recited value (i.e., having the same function or result). In many instances, the term “about” may include numbers that are rounded to the nearest significant figure. In certain instances, the term “about” may be used to denote values falling within ±20% of the recited values, e.g., within ±15%, ±10%, ±7.5%, ±5%, ±4%, ±3%, ±2% or ±1% of the recited values.

The term “baseline” means the first measurement of the targeted variable just before the administration of the studied therapy.

Unless the context requires otherwise, the terms “comprise,” “comprises,” and “comprising” are used on the basis and clear understanding that they are to be interpreted inclusively, rather than exclusively, such that they indicate the inclusion of the recited feature but without excluding one or more other such features.

The term “patient”, “subject”, “individual” and the like refers to humans.

The term “treating” used herein means that the administration of a monoclonal antibody that binds to PD-1 (e.g., mAb2) and/or a monoclonal antibody that bind α4β7 (e.g., mAb1) is sufficient to reduce or ameliorate the severity and/or duration of HIV infection, or one or more symptoms thereof.

As used herein, the term “effective amount” or “therapeutically effective amount” refers to the amount of a monoclonal antibody that binds to PD-1 (e.g., mAb2) and/or a monoclonal antibody that bind α4β7 (e.g., mAb1) that is sufficient to reduce or ameliorate the severity and/or duration of HIV infection, or one or more symptoms thereof. The therapeutically effective amount of a monoclonal antibody that binds to PD-1 (e.g., mAb2) and/or a monoclonal antibody that bind α4β7 (e.g., mAb1) may, for example, reduces the total HIV viral load to less than about 5,000 copies/mL, less than 1,000 copies/mL, or less than 200 copies/mL without antiretroviral treatment. The therapeutically effective amount of a monoclonal antibody that binds to PD-1 (e.g., mAb2) and/or a monoclonal antibody that bind α4β7 (e.g., mAb1) may, for example, reduces the total HIV viral load to less than about 5,000 copies/mL, less than 1,000 copies/mL, or less than 200 copies/mL without antiretroviral treatment at week 24 or after about 25 weeks. The therapeutically effective amount of a monoclonal antibody that binds to PD-1 (e.g., mAb2) and/or a monoclonal antibody that bind α4β7 (e.g., mAb1) may, for example, reduces the total HIV viral load to less than about 5,000 copies/mL, less than 1,000 copies/mL, or less than 200 copies/mL without antiretroviral treatment for at least one year.

The term “and/or” as used in a phrase such as “A and/or B” herein is intended to mean “A and B”, “A or B”, “A” or “B”.

The term “ATI,” as used herein means analytical treatment interruption.

The term “mAb1,” as used herein, refers to a monoclonal antibody which is reactive to human α4β7 (integrin), or anti-α4β7, as described herein (e.g., as described in the embodiments and Examples).

The term “mAb2,” as used herein, means a monoclonal antibody which is reactive to human PD-1 receptor, or anti-PD-1, as described herein (e.g., as described in the embodiments and Examples).

The use of the terms “a” and “an” and “the” and similar referents in the context of describing the invention (especially in the context of the following claims) are to be construed to cover both the singular and the plural, unless otherwise indicated herein or clearly contradicted by context.

### ADDITIONAL EXEMPLARY EMBODIMENTS

Embodiment 1. A method of treating human immunodeficiency virus (HIV) infection in a patient infected with HIV-1, the method comprising administering to the patient a therapeutically effective amount of a monoclonal antibody which binds to human programmed death receptor 1 (PD-1).

Embodiment 2. The method of embodiment 1, wherein the monoclonal antibody which binds to PD-1 is mAb2 which comprises (i) a heavy chain variable region (VH) comprising three CDRs: VH CDR #1, VH CDR #2, and VH CDR #3; and (ii) a light chain variable region (VL) comprising three CDRs: VL CDR #1, VL CDR #2, and VL CDR #3, wherein: VH CDR #1 is GYTFTHYGMN (SEQ ID NO:1); VH CDR #2 is WVNTYTGEPTYADDFKG (SEQ ID NO:2); VH CDR #3 is EGEGLGFGD (SEQ ID NO:3); VL CDR #1 is RSSQSIVHSHGDTYLE (SEQ ID NO:4); VL CDR #2 is KVSNRFS (SEQ ID NO:5); and VL CDR #3 is FQGSHIPVT (SEQ ID NO:6).

Embodiment 3. The method of embodiment 2, wherein the mAb2 comprises (i) a VH comprising the amino acid sequence of SEQ ID NO: 7; and (ii) a VL comprising the amino acid sequence of SEQ ID NO: 8.

Embodiment 4. The method of embodiment 2 or 3, wherein the mAb2 comprises (i) a heavy chain (HC) comprising the amino acid sequence of SEQ ID NO: 9; and (ii) a light chain (LC) comprising the amino acid sequence of SEQ ID NO: 10.

Embodiment 5. The method of embodiment 2 or 3, wherein the mAb2 comprises (i) a heavy chain (HC) comprising the amino acid sequence of SEQ ID NO: 20; and (ii) a light chain (LC) comprising the amino acid sequence of SEQ ID NO: 10.

Embodiment 6. The method of any one of embodiments 1-5, wherein the monoclonal antibody which binds to PD-1 is administered to the patient in an amount of from about 10 mg to about 20 mg.

Embodiment 7. The method of embodiment 6, wherein the monoclonal antibody that binds to PD-1 is administered to the patient in an amount of about 10 mg.

Embodiment 8. The method of embodiment 6, wherein the monoclonal antibody that binds to PD-1 is administered to the patient in an amount of about 20 mg.

Embodiment 9. The method of any one of embodiments 1-8, wherein the monoclonal antibody that binds to PD-1 is administered to the patient via subcutaneous (SC) injection or intravenous (IV) infusion.

Embodiment 10. The method of any one of embodiments 1-9, wherein the monoclonal antibody that binds to PD-1 is administered to the patient via IV infusion in an amount of about 10 mg at Week 0 (Day 1), Week 2 (Day 15), Week 4 (Day 29), and Week 6 (Day 43).

Embodiment 11. The method of any one of embodiments 1-9, wherein the monoclonal antibody that binds to PD-1 is administered to the patient via SC injection in an amount of about 20 mg at Week 0 (Day 1), Week 2 (Day 15), Week 4 (Day 29), and Week 6 (Day 43).

Embodiment 12. The method of any one of embodiments 1-11, wherein after about 25 weeks the patient achieves an HIV viral load of less than about 5,000 copies/mL.

Embodiment 13. The method of embodiment 12, wherein the HIV-1 viral load of less than about 5,000 copies/mL is achieved without antiretroviral treatment.

Embodiment 14. The method of any one of embodiments 1-13, wherein the HIV-1 viral load of less than 1,000 copies/mL is achieved without antiretroviral treatment.

Embodiment 15. The method of any one of embodiments 1-14, wherein the HIV-1 viral load of less than 200 copies/mL is achieved without antiretroviral treatment.

Embodiment 16. The method of any one of embodiments 1-14, wherein the HIV-1 viral load is less than about 1,000 copies/mL for at least one year.

Embodiment 17. The method of any one of embodiments 1-16, wherein the HIV-1 viral load is less than about 200 copies/mL for at least one year.

Embodiment 18. A method of treating human immunodeficiency virus (HIV) infection in a patient infected with HIV-1, the method comprising administering to the patient a therapeutically effective amount of a monoclonal antibody which binds to human α4β7.

Embodiment 19. The method of embodiment 18, wherein the monoclonal antibody which binds to α4β7 is mAb1 which comprises i) a heavy chain variable region (VH) comprising three CDRs: VH CDR #1, VH CDR #2, and VH VDR #3; and (ii) a light chain variable region (VL) comprising three CDRs: VL CDR #1, VL CDR #2, and VL VDR #3, wherein: VH CDR #1 is GFNIKNTYMH (SEQ ID NO:11); VH CDR #2 is RIDPAKGHTEYAPKFLG (SEQ ID NO:12); VH CDR #3 is VDV; VL CDR #1 is HASQDISDNIG (SEQ ID NO:13); VL CDR #2 is HGTNLED (SEQ ID NO:14); and VL CDR #3 is VQYAQFPWT (SEQ ID NO:15).

Embodiment 20. The method of embodiment 19, wherein the monoclonal antibody which binds to α4β7 is mAb1 which comprises (i) a VH comprising the amino acid sequence of SEQ ID NO: 16; and (ii) a VL comprising the amino acid sequence of SEQ ID NO: 17.

Embodiment 21. The method of embodiment 19 or 20, wherein the mAb1 comprises (i) a heavy chain (HC) comprising the amino acid sequence of SEQ ID NO: 18; and (ii) a light chain (LC) comprising the amino acid sequence of SEQ ID NO: 19.

Embodiment 22. The method of embodiment 19 or 20, wherein the mAb1 comprises (i) a heavy chain (HC) comprising the amino acid sequence of SEQ ID NO: 21; and (ii) a light chain (LC) comprising the amino acid sequence of SEQ ID NO: 19.

Embodiment 23. The method of any one of embodiments 18-22, wherein the monoclonal antibody which binds to α4β7 is administered in an amount of from about 800 mg to about 1600 mg.

Embodiment 24. The method of embodiment 23, wherein the monoclonal antibody which binds to α4β7 is administered in an amount of about 800 mg.

Embodiment 25. The method of embodiment 23, wherein the monoclonal antibody which binds to α4β7 is administered in an amount of about 1600 mg.

Embodiment 26. The method of any one of embodiments 18-25, wherein the monoclonal antibody which binds to α4β7 is administered via subcutaneous (SC) injection or intravenous (IV) infusion.

Embodiment 27. The method of any one of embodiments 18-26, wherein the monoclonal antibody which binds to α4β7 is administered via IV infusion in an amount of about 800 mg at Week 0 (Day 1), Week 4 (Day 29), and Week 8 (Day 57).

Embodiment 28. The method of any one of embodiments 18-26, wherein the monoclonal antibody which binds to α4β7 is administered via IV infusion in an amount of about 1600 mg at Week 0 (Day 1), Week 4 (Day 29), and Week 8 (Day 57).

Embodiment 29. The method of any one of embodiments 18-28, wherein after about 25 weeks the patient achieves an HIV viral load of less than about 5,000 copies/mL.

Embodiment 30. The method of embodiment 29, wherein the HIV-1 viral load of less than about 5,000 copies/mL is achieved without antiretroviral treatment.

Embodiment 31. The method of any one of embodiments 18-30, wherein the HIV-1 viral load of less than 1,000 copies/mL is achieved without antiretroviral treatment.

Embodiment 32. The method of any one of embodiments 18-31, wherein the HIV-1 viral load of less than 200 copies/mL is achieved without antiretroviral treatment.

Embodiment 33. The method of any one of embodiments 18-31, wherein the HIV-1 viral load is less than about 1,000 copies/mL for at least one year.

Embodiment 34. The method of any one of embodiments 18-33, wherein the HIV-1 viral load of less than about 200 copies/mL for at least one year.

Embodiment 35. A method of treating human immunodeficiency virus (HIV) infection in a patient infected with HIV-1, the method comprising: administering to the patient a therapeutically effective amount of a first monoclonal antibody which binds to human PD-1; and administering to the patient a therapeutically effective amount of a second monoclonal antibody which binds to human α4β7.

Embodiment 36. The method of embodiment 35, wherein the first monoclonal antibody is mAb2 which comprises (i) a VH comprising three CDRs: VH CDR #1, VH CDR #2, and VH CDR #3; and (ii) a VL comprising three CDRs: VL CDR #1, VL CDR #2, and VL CDR #3, wherein: VH CDR #1 is GYTFTHYGMN (SEQ ID NO:1); VH CDR #2 is WVNTYTGEPTYADDFKG (SEQ ID NO:2); VH CDR #3 is EGEGLGFGD (SEQ ID NO:3); VL CDR #1 is RSSQSIVHSHGDTYLE (SEQ ID NO:4); VL CDR #2 is KVSNRFS (SEQ ID NO:5); and VL CDR #3 is FQGSHIPVT (SEQ ID NO:6).

Embodiment 37. The method of embodiment 36, wherein the mAb2 comprises (i) a VH comprising the amino acid sequence of SEQ ID NO: 7; and (ii) a VL comprising the amino acid sequence of SEQ ID NO: 8.

Embodiment 38. The method of embodiment 36 or 37, wherein the mAb2 comprises (i) a heavy chain (HC) comprising the amino acid sequence of SEQ ID NO: 9; and (ii) a light chain (LC) comprising the amino acid sequence of SEQ ID NO: 10.

Embodiment 39. The method of embodiment 36 or 37, wherein the mAb2 comprises (i) a heavy chain (HC) comprising the amino acid sequence of SEQ ID NO: 20; and (ii) a light chain (LC) comprising the amino acid sequence of SEQ ID NO: 10.

Embodiment 40. The method of any one of embodiments 35-39, wherein the second monoclonal antibody is mAb1 which comprises i) a VH comprising three CDRs: VH CDR #1, VH CDR #2, and VH CDR #3; and (ii) a VL comprising three CDRs: VL CDR #1, VL CDR #2, and VL CDR #3, wherein: VH CDR #1 is GFNIKNTYMH (SEQ ID NO:11); VH CDR #2 is RIDPAKGHTEYAPKFLG (SEQ ID NO:12); VH CDR #3 is VDV, VL CDR #1 is HASQDISDNIG (SEQ ID NO:13); VL CDR #2 is HGTNLED (SEQ ID NO:14); and VL CDR #3 is VQYAQFPWT (SEQ ID NO:15).

Embodiment 41. The method of embodiment 40, wherein the mAb1 comprises (i) a VH comprising the amino acid sequence of SEQ ID NO: 16; and (ii) a VL comprising the amino acid sequence of SEQ ID NO: 17.

Embodiment 42. The method of embodiment 40 or 41, wherein the mAb1 comprises (i) a heavy chain (HC) comprising the amino acid sequence of SEQ ID NO: 18; and (ii) a light chain (LC) comprising the amino acid sequence of SEQ ID NO: 19.

Embodiment 43. The method of embodiment 40 or 41, wherein the mAb1 comprises (i) a heavy chain (HC) comprising the amino acid sequence of SEQ ID NO: 21; and (ii) a light chain (LC) comprising the amino acid sequence of SEQ ID NO: 19.

Embodiment 44. The method of any one of embodiments 35-43, wherein the first monoclonal antibody is administered via intravenous infusion or via subcutaneous injection.

Embodiment 45. The method of any one of embodiments 35-44, wherein the second monoclonal antibody is administered via intravenous infusion or via subcutaneous injection.

Embodiment 46. The method of any one of embodiments 35-45, wherein the first monoclonal antibody is administered in an amount of about 10 mg.

Embodiment 47. The method of any one of embodiments 35-45, wherein the first monoclonal antibody is administered in an amount of about 20 mg.

Embodiment 48. The method of any one of embodiments 35-47, wherein the second monoclonal antibody is administered in an amount of about 800 mg.

Embodiment 49. The method of any one of embodiments 35-47, wherein the second monoclonal antibody is administered in an amount of about 1600 mg.

Embodiment 50. The method of any one of embodiments 35-49, wherein the first monoclonal antibody and the second monoclonal antibody are administered at the same time.

Embodiment 51. The method of any one of embodiments 35-49, wherein the first monoclonal antibody and the second monoclonal antibody are administered sequentially.

Embodiment 52. The method of any one of embodiments 35-51, wherein the first monoclonal antibody is administered once every 2 weeks for about 6 weeks.

Embodiment 53. The method of any one of embodiments 35-52, wherein the second monoclonal antibody is administered once every 4 weeks for about 8 weeks.

Embodiment 54. The method of any one of embodiments 35-53, wherein the method comprises: administering the first monoclonal antibody in an amount of about 10 mg or 20 mg on Week 0 (Day 1), Week 2 (Day 15), Week 4 (Day 29), and Week 6 (Day 43); and administering the second monoclonal antibody in an amount of about 800 mg or 1600 mg on Week 0 (Day 1), Week 4 (Day 29), and Week 8 (Day 57).

Embodiment 55. The method of any one of embodiments 35-54, wherein the method comprises: administering the first monoclonal antibody via IV infusion in an amount of about 10 mg on Week 0 (Day 1), Week 2 (Day 15), Week 4 (Day 29), and Week 6 (Day 43); and administering the second monoclonal antibody via IV infusion in an amount of about 800 mg on Week 0 (Day 1), Week 4 (Day 29), and Week 8 (Day 57).

Embodiment 56. The method of any one of embodiments 35-54, wherein the method comprises: administering the first monoclonal antibody via IV infusion in an amount of about 10 mg on Week 0 (Day 1), Week 2 (Day 15), Week 4 (Day 29), and Week 6 (Day 43); and administering the second monoclonal antibody via IV infusion in an amount of about 1600 mg on Week 0 (Day 1), Week 4 (Day 29), and Week 8 (Day 57).

Embodiment 57. The method of any one of embodiments 35-54, wherein the method comprises: administering the first monoclonal antibody via SC injection in an amount of about 20 mg on Week 0 (Day 1), Week 2 (Day 15), Week 4 (Day 29), and Week 6 (Day 43); and administering the second monoclonal antibody via IV infusion in an amount of about 800 mg on Week 0 (Day 1), Week 4 (Day 29), and Week 8 (Day 57).

Embodiment 58. The method of any one of embodiments 35-54, wherein the method comprises: administering the first monoclonal antibody via SC injection in an amount of about 20 mg on Week 0 (Day 1), Week 2 (Day 15), Week 4 (Day 29), and Week 6 (Day 43); and administering the second monoclonal antibody via IV infusion in an amount of about 1600 mg on Week 0 (Day 1), Week 4 (Day 29), and Week 8 (Day 57).

Embodiment 59. The method of any one of embodiments 35-58, wherein the time to viral rebound following interruption of antiretroviral treatment is increased compared with treatment prior to the administration of both anti-PD-1 and anti-α4β7 antibodies, wherein the viral rebound is indicated by a viral load greater than about 1000 copies/mL.

Embodiment 60. The method of any one of embodiments 35-59, wherein the peak viral load at viral rebound before starting antiretroviral treatment is decreased compared with treatment prior to the administration of both anti-PD-1 and anti-α4β7 antibodies, wherein the viral rebound is indicated by a viral load greater than about 1000 copies/mL.

Embodiment 61. The method of any one of embodiments 35-60, wherein after about 25 weeks the patient achieves an HIV viral load of less than about 5,000 copies/mL.

Embodiment 62. The method of embodiment 61, wherein the HIV-1 viral load of less than about 5,000 copies/mL is achieved without antiretroviral treatment.

Embodiment 63. The method of any one of embodiments 35-62, wherein the HIV-1 viral load of less than 1,000 copies/mL is achieved without antiretroviral treatment.

Embodiment 64. The method of any one of embodiments 35-63, wherein a HIV-1 viral load of less than about 1000 copies/mL is achieved without antiretroviral treatment at week 24.

Embodiment 65. The method of any one of embodiments 35-64, wherein the HIV-1 viral load of less than 200 copies/mL is achieved without antiretroviral treatment.

Embodiment 66. The method of any one of embodiments 35-64, wherein the HIV-1 viral load of less than about 1,000 copies/mL for at least one year.

Embodiment 67. The method of any one of embodiments 35-66, wherein the HIV-1 viral load of less than about 200 copies/mL for at least one year.

Embodiment 68. The methods of any one of embodiments 1-67, wherein the patient is an adult patient.

Embodiment 69. The methods of any one of embodiments 1-68, wherein the patient is living with HIV who is virologically suppressed on anti-retroviral therapy (ART) undergoing analytical treatment interruption.

### EXAMPLES

In order that the invention described herein may be more fully understood, the following examples are set forth.

### Example 1 Study A

This Examples describes a study of single dose subcutaneous (SC) and intravenous (IV) administration of anti-PD-1 monoclonal antibody mAb2 in adult people living with HIV (PLWH).

### Study Objectives:

The objectives of this study included: 1) evaluating the safety and tolerability of SC and IV administration of mAb2 having heavy chains shown as SEQ ID NO:9 and may also have a C-terminal lysine truncated heavy chain of SEQ ID NO: 20 and light chains shown as SEQ ID NO:10 single doses in subjects who were virologically suppressed on ART, and 2) evaluating the PK and immunogenicity after SC and IV administration of mAb2 single doses in subjects who were virologically suppressed on ART.

Additional objectives for this study included evaluating receptor saturation in peripheral blood cells, evaluating the impact of mAb2 on the immune (including HIV specific) response, and evaluating mAb2 in latency reversal of the viral reservoir (cells harboring pro-viral deoxyribonucleic acid).

### Background and Rationale:

In this Phase 1b study, the safety, PK, and pharmacodynamics (PD) of subcutaneous (SC) and intravenous (IV) administration of single doses of mAb2, were evaluated in adult people living with human immunodeficiency virus (PLWH) who were virologically suppressed on combination anti-retroviral therapy. This study also enabled a direct comparison with respect to safety, PK, and PD between the IV and SC administrative routes of mAb2.

### Investigational Plan:

This was a multi-center, Phase 1b, randomized, double-blind and placebo-controlled single-dose study to evaluate the safety, PK, and PD of mAb2 SC and IV administration in adult PLWH who were virologically suppressed on stable ART.

The subjects received a single dose of the study drug followed by a 24-week post treatment period. Subjects remained on their ART regimen for the entire study. Approximately 32 subjects who met eligibility criteria were randomized in parallel (1:1:1:1 ratio) to 1 of 4 treatment groups:

Group 1: Placebo SC+Placebo IV (n=8)

Group 2: mAb2 10 mg SC+Placebo IV (n=8)

Group 3: mAb2 20 mg SC+Placebo IV (n=8)

Group 4: Placebo SC+mAb2 10 mg IV (n=8)

On the day of dosing, subjects received the study drug by SC route of administration (mAb2 or placebo) in the abdomen, followed by IV route of administration (mAb2 or placebo) to maintain blinding. Week 0 (Day 1) blood samples were collected prior to SC dosing and after dosing with respect to timing of completing IV administration.

### Key Eligibility Criteria:

1. Subjects were 18 to 65 years of age, with a condition of generally good health, body mass index ≥18.0 to <35.0 kg/m².
2. Laboratory values met acceptable criteria.
3. Subjects were HIV-1 infected and used antiretroviral therapy (ART) for at least 12 months prior to screening and were on a current ART regimen for at least 8 weeks prior to screening (regimen could not include Celsentri® (Maraviroc, Pfizer, New York, NY, USA)).
4. CD4 cell count was ≥450 cells/μL at screening and during the 12 months prior to screening.
5. Plasma HIV-1 RNA was below the lower limit of quantification at screening and at least 6 months prior to screening.
6. Subjects must not have had signs/symptoms associated with SARS CoV-2 infection.
7. Subjects had absence of current SARS CoV-2 infection by any viral nucleic acid test (polymerase chain reaction) completed within 7 days prior to the Week 0 (Day 1) dose.
8. Subjects must not have had history or ongoing diagnosis of acquired immunodeficiency syndrome (AIDS)-defining illness.
9. Subjects must not have had history of or active immunodeficiency (other than HIV).
10. Subjects must not have had active autoimmune disease or history of autoimmune disease that has required systemic treatment.
11. Subjects must not have had prior therapy or exposure to mAb2 or any other immune checkpoint inhibitor (e.g., anti-PD-1, anti-PD-L1, anti-PD-L2, anti-CTLA-4).
12. Subjects must not have had clinically significant medical disorders that might expose the subjects to undue risk of harm, confound study outcomes, or prevent the subject from completing the study, including but not limited to significant or unstable cardiac, neurologic, or pulmonary disease, chronic active infectious disease except for HIV, chronic liver disease, poorly controlled diabetes mellitus, and history of Stevens-Johnson Syndrome.
13. Subjects must not have had active or suspected malignancy or history of malignancy (other than basal cell skin cancer or cervical carcinoma in situ) in the past 5 years.
14. Subjects must not have had history of or active tuberculosis (TB) at screening.
15. Subjects must not have had history of positive TB skin test or interferon gamma release assay (IGRA) which is considered clinically significant by the investigator.
16. Subjects must not have had known psychiatric or substance abuse disorders that would interfere with adherence to study requirements.
17. Subjects must have agreed to use an effective barrier method of protection (male and/or female condom) during sexual activity from Week 0 (Day 1) through last study visit for the purposes of prevention of HIV transmission.
18. Female subjects must not have been pregnant, breastfeeding, or considering becoming pregnant during the study.
19. Subjects must not have received immunomodulatory or immunosuppressive (including IV or orally administered [PO] steroids at any dose, but excluding steroids that are inhaled, topical or via local injection) therapy within 24 weeks prior to the first dose of study drug.

### Treatment Duration:

Eligible subjects received a single dose of study drug (mAb2 or placebo) via IV administration and/or a single dose of study drug (mAb2 or placebo) via SC administration.

### Evaluation:

The study results were evaluated by specific endpoints described below. Adverse Event (AE), Immune-Related Adverse Event (IRAE), and PK Endpoints:

The AE endpoints of this study were drug-related Grade 3 or higher adverse events (AEs), study drug-related IRAEs (immune-related adverse events), and pharmacokinetics.

### Safety Endpoints:

Safety endpoints included frequency and severity of adverse events (AEs) and adverse events of special interest (AESIs), including immune related adverse events (IRAEs), infusion-related reactions, injection site reactions and hepatotoxicity. Safety evaluations included vital sign measurements, physical examinations, and clinical laboratory testing (including hematology and metabolic panels, thyroid function, hemolysis, plasma HIV-1 ribonucleic acid (RNA), and CD4+, T cell, B cell, and NK cell counts [TBNK panel]) throughout the study. Virologic failures were also evaluated.

### Pharmacokinetic Endpoints:

The pharmacokinetic endpoints for mAb2 were the maximum observed plasma concentration (Cₘₐₓ), time to Cₘₐₓ (Tₘₐₓ) following administration of mAb2, area under the plasma concentration-time curve (AUC), and terminal phase elimination half-life (t_{1/2}) after the last dose.

Immunogenicity of mAb2 was assessed by using a tiered approach for detecting anti-drug antibodies (ADA). Neutralizing anti-drug antibodies (nAb) may have been evaluated if useful for interpretation of results.

### Pharmacodynamic Endpoints:

1. Peripheral PD-1 receptor saturation (%) on CD4+ and CD8+ T cell subsets pre- and post-study drug treatment.
2. Immunomodulatory effects pre- and post-study drug treatment.
3. Induction of proliferation and activation markers.
4. Ex-vivo HIV-specific T cell response.
5. Latency reversal: changes in HIV transcription pre- and post-study drug treatment.

### Results:

The adverse events observed in the study are shown in Table 1A. There were no reported deaths, no drug-related serious adverse event (SAE) and no AE ≥Grade 3 related to the study drug. There were no infusion-related reactions or injection site reactions reported. There was no drug related hepatic AESI.

All subjects completed the study. The severity and types of AEs reported were expected based on mode of action, consistent with those occurring in this study population. No new safety signals were observed.

TABLE 1A
MAb2 in PLWH: Overview of Subjects with Treatment
Emergent Adverse Events in Study A.

| | 10 mg SC | 20 mg SC | 10 mg IV | Placebo |
|---|---|---|---|---|
| n (% of patients) | (n = 8) | (n = 8) | (n = 8) | (n = 8) |
| Any AE | 6 (75%) | 5 (62.5%) | 6 (75%) | 5 (62.5%) |
| Drug-related AE* | 1 (12.5%) | 2 (25%) | 4 (50%) | 2 (25%) |
| SAE | 0 | 0 | 0 | 1 (12.5%) |
| Drug-related SAE | 0 | 0 | 0 | 0 |
| AE leading to death | 0 | 0 | 0 | 0 |
| AE grade 3 or higher | 0 | 0 | 1 (12.5%) | 1 (12.5%) |
| Hepatic-related AESI | 0 | 0 | 1 (12.5%) | 0 |
| IRAEs (any grade) | 0 | 1 (12.5%) | 0 | 0 |
| | | Grade 2 | | |

As assessed by the principal investigator.

For the 20 mg SC single dose, one IRAE was observed, a lichenoid keratosis, with an onset on day 15-day 51. The highest-grade AE observed was grade 2, which resolved. This AE was considered possibly related to the study drug. Overall, no dose-limiting toxicity was observed in this study.

Effects of mAb2 Treatments on HIV Disease Status:

HIV-RNA was monitored as per protocol at Week 0 (Day 1) and Weeks 1, 2, 4, 8, 12, and 24 (Days 8, 15, 29, 57, 85, 169). Subjects have not stopped their ART in this study. There was no virological failure during the study period.

### Laboratory Summary:

There was no clinically significant trend observed for changes in CD4 T cell count or hematology, or urinalysis parameters.

Three subjects, due to elevated creatine met criteria for potentially clinically significant chemistry values with Grade ≥2, (2 from the placebo group and 1 from the 10 mg IV group). None of those creatinine elevations were considered clinically significant as an AE.

In addition, one other subject experienced a Grade 1 AE of increased blood creatinine (creatinine elevations Grade 1 by Division of AIDS (DAIDS)) attributed to dehydration that resolved with hydration treatment in 29 days. This AE was not considered as possibly related to the study drug by the PI.

One subject who received a 10 mg IV dose had a reversible high Thyroid-stimulating hormone (TSH) at Week 0 (Day 1) (predose) and Week 6 (Day 43) with Triiodothyronine (T3) and thyroxine (T4) in normal range. Two subjects in the placebo group and one subject in 10 mg IV group had a single measurement of lower TSH. One subject had a single high fasting glucose measurement. None of these laboratory abnormalities were considered as clinically significant as an AE or IRAE.

### Vital Signs Summary

No clinically important trends in vital signs were observed, and no subjects met the criteria for potentially significant values during the treatment period. There were no vital signs-related Adverse Events reported

### Electrocardiogram (ECG) Summary

ECGs were evaluated for entry into the study at baseline and could be performed as needed for any post-baseline safety evaluation. No subject necessitated a post-baseline ECG evaluation. There were no cardiac or ECG-related AEs reported.

Assessment of mAb2 Exposure-Response Relationships for Safety Immune-Related Adverse Events

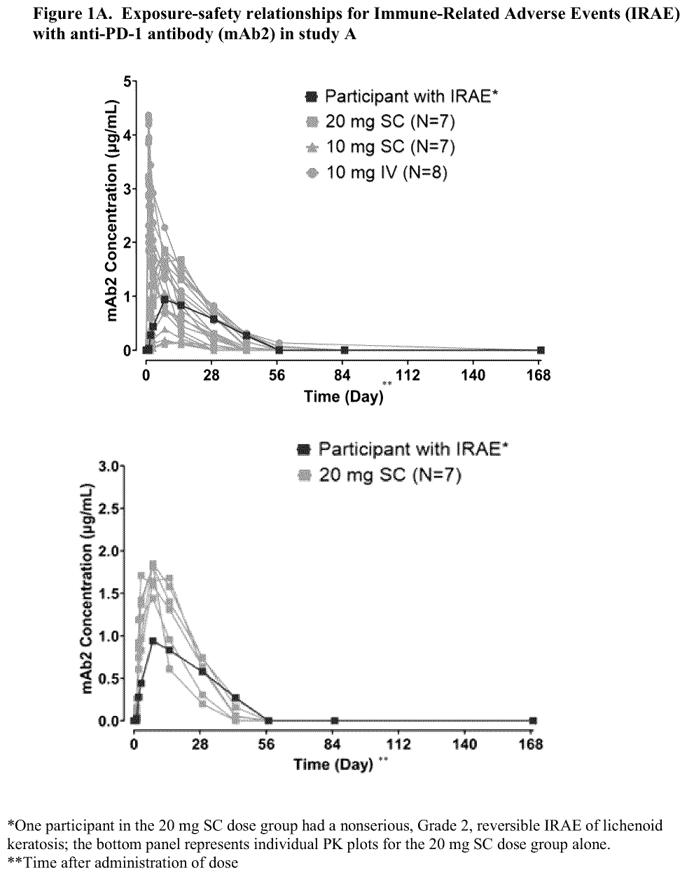

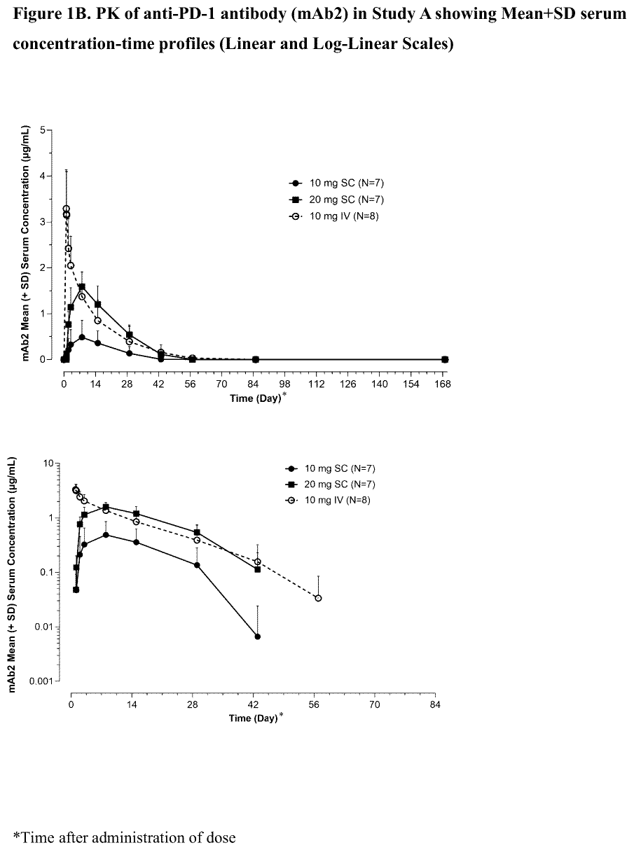

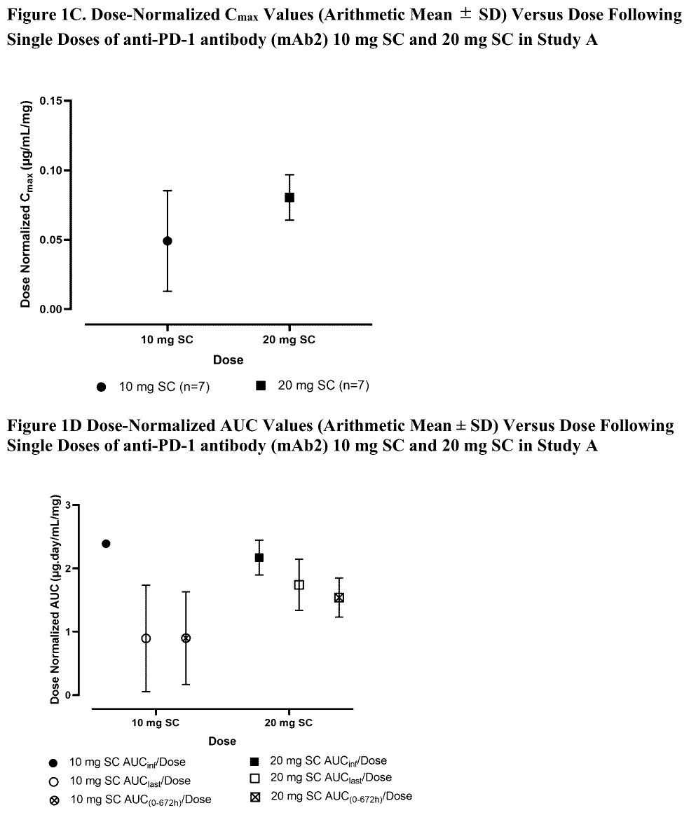

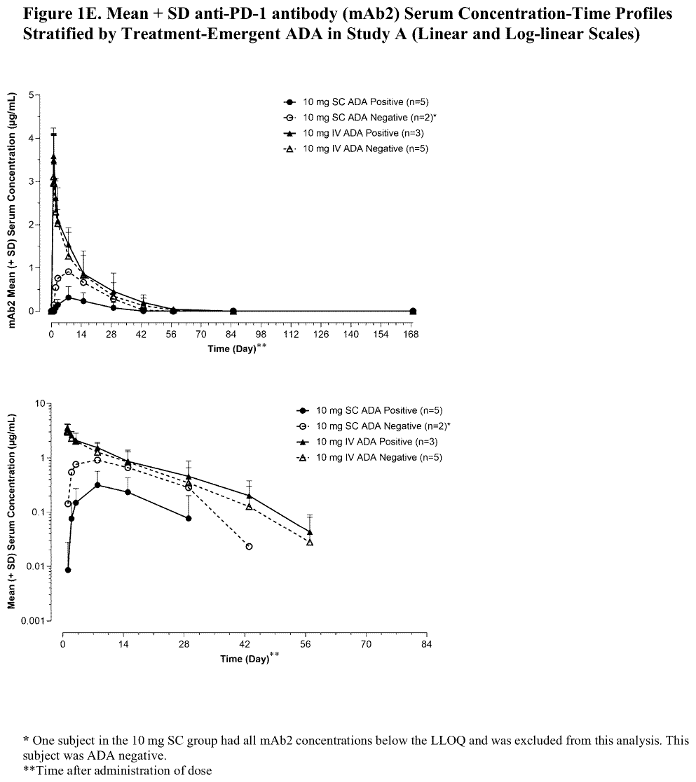

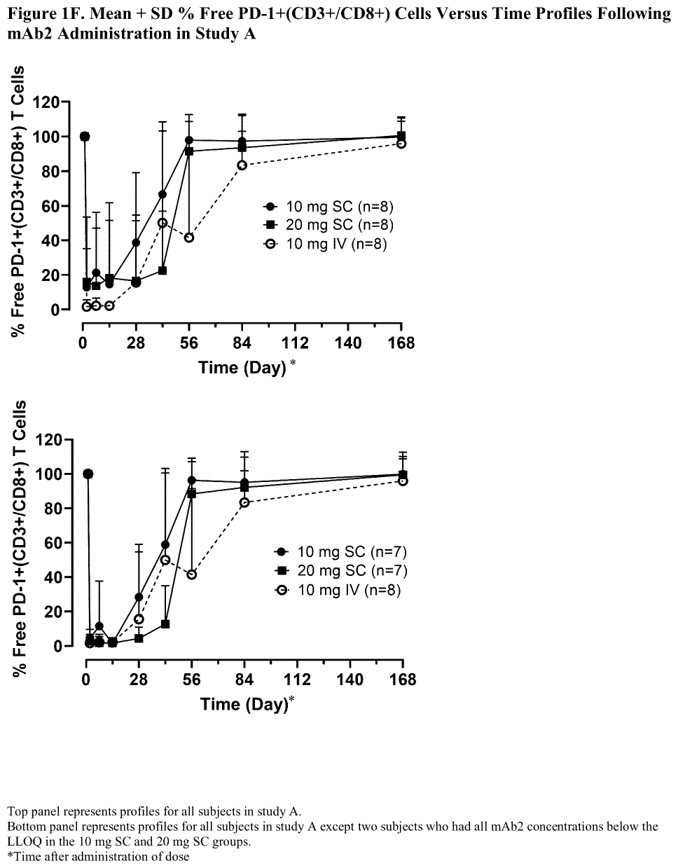

Exposure-response relationships for safety in Study A were evaluated by comparing the exposures of subjects who demonstrated safety signals (IRAEs) with those who did not. Individual plots of mAb2 PK stratified by safety (IRAEs) are depicted in FIG. 1A. No apparent trends or exposure-response relationships were identified. The exposures of the subject with a safety signal (IRAE) in Study A were comparable with the exposures of the remaining subjects in their dose group, i.e., 20 mg SC.

PK of mAb2 in Study A

PK of mAb2 in Study A was evaluated and is presented in FIG. 1B. In study A, one subject each in the 10 mg SC and 20 mg SC dose groups had all mAb2 concentrations below the lower limit of quantitation (LLOQ) and were excluded from this analysis. A summary of the PK parameters of mAb2 in Study A is shown in Table 1B below.

TABLE 1B
Geometric Mean (Mean, CV %) Pharmacokinetic Parameters of mAb2 in Study A

| | Group 2 | | Group 3 | | Group 4 | |
|---|---|---|---|---|---|---|
| | mAb2 | | mAb2 | | mAb2 | |
| | 10 mg SC | | 20 mg SC | | 10 mg IV | |
| PK Parameters (unit) | (N = 7)^{c} | | (N = 7)^{c} | | (N = 8) | |
| Cₘₐₓ (μg/mL) | 0.372 | (0.49, 74.0) | 1.57 | (1.61, 20.3) | 3.20 | (3.30, 26.0) |
| Tₘₐₓ^{a} (h) | 194.9 | (146.8-311.6) | 146.5 | (46.3-336) | 0.25 | (0.25-4.0) |
| t_{1/2}^{b} (day) | 6.47 | (0)^{d} | 9.32 | (4.03)^{e} | 8.02 | (3.79) |
| AUC_{0-672 h} (μg · day/mL) | 6.49 | (9.00, 81.5) | 30.2 | (30.8, 20.1) | 28.0 | (30.2, 40.5) |
| AUCₜ (μg · day/mL) | 5.69 | (8.95, 93.9) | 34.0 | (34.8, 23.3) | 30.4 | (34.2, 49.4) |
| AUC_{inf} (μg · day/mL) | 23.9 | (23.9, NR)^{d} | 43.1 | (43.3, 12.7)^{f} | 32.4 | (36.3, 48.8) |
| Cₘₐₓ/dose (μg/mL/mg) | 0.037 | (0.049, 74.0) | 0.079 | (0.08, 20.3) | 0.32 | (0.33, 26.0) |
| AUC_{0-672 h}/dose | 0.649 | (0.9, 81.5) | 1.51 | (1.54, 20.1) | 2.80 | (3.02, 40.5) |
| (μg · day/mL/mg) | | | | | | |
| AUCₜ/dose (μg · day/mL/mg) | 0.569 | (0.895, 93.9) | 1.70 | (1.74, 23.3) | 3.04 | (3.42, 49.4) |
| AUC_{inf}/dose | 2.39 | (2.39, NR)^{d} | 2.15 | (2.17, 12.7)^{f} | 3.24 | (3.63, 48.8) |
| (μg · day/mL/mg) | | | | | | |

AUC = area under the concentration curve;
AUC_{0-672 h} = AUC from time 0 to 672 hours;
AUC_{inf} = AUC from time 0 to infinity;
AUCₜ = AUC from time 0 until the last observable concentration;
Cₘₐₓ = maximum observed concentration;
CV = coefficient of variation;
IV = intravenous;
LLOQ = lower limit of quantitation;
NR = not reported;
PK = pharmacokinetic;
SC = subcutaneous;
t_{1/2} = apparent elimination half-life;
Tₘₐₓ = time to maximum observed concentration
^{a}Tₘₐₓ presented as median and range.
^{b}t_{1/2} presented as harmonic mean and pseudo standard deviation.
^{c}One subject each in the 10 mg SC and 20 mg SC dose groups had all mAb2 concentrations below the LLOQ and were excluded from the analysis.
^{d}N = 1
^{e}N = 4
^{f}N = 3
Note:
The protocol-defined IV infusion duration was at least 30 minutes.

Following administration of single doses of mAb2 10 mg SC, 20 mg SC, and 10 mg IV, the geometric mean Cₘₐₓ was 0.372, 1.57, and 3.2 μg/mL, respectively. Following administration of single doses of mAb2 10 mg SC, 20 mg SC, and 10 mg IV, the maximum mAb2 serum concentrations were observed at approximately 147 to 312 hours (6 to 13 days), 46 to 336 hours (2 to 14 days), and 0.25 to 4.0 hours, respectively. The apparent elimination half-life for the 10 mg SC, 20 mg SC, and 10 mg IV doses was approximately 6.5, 9, and 8 days, respectively. Dose proportionality of mAb2 in the SC dose groups was investigated using an ANCOVA of the log-transformed dose-normalized Cₘₐₓ, AUCₜ, AUC_{0-672 h}, and AUC_{inf} values. With the exception of AUC_{inf}, there was a significant difference in mAb2 dose-normalized Cₘₐₓ, AUCₜ, and AUC_{0-672 h} for the 20 mg SC dose compared to the 10 mg SC dose. Based on the ANCOVA on dose-normalized Cₘₐₓ, AUCₜ and AUC_{0-672 h}, these results suggest that exposures following 20 mg SC dosing were more than dose-proportional relative to exposures following 10 mg SC dosing. The AUC_{inf} was calculable for 1 out of 7 subjects evaluable for PK in the 10 mg SC group. The variability in exposures following 10 mg SC dosing was >70% CV as compared to 20 mg SC dosing (<25% CV). FIGS. 1C and 1D depict dose-normalized Cₘₐₓ and AUC values versus dose following single doses of anti-PD1 antibody (mAb2) 10 mg SC and 20 mg SC.

Bioavailability Assessment for mAb2 SC Regimens

Bioavailability following SC administration of mAb2 was assessed using ANCOVA by comparing the exposure data from the 10 mg SC and 10 mg IV dose groups and from 20 mg SC and 10 mg IV dose groups. The ratio of the central values and the 90% confidence intervals for AUCₜ, and AUC_{inf} (or the respective dose-normalized values for 20 mg SC) are presented in Table 1C.

Following SC administration of 10 mg mAb2, the exposure (AUC_{inf}) was approximately 53% of the exposure following 10 mg IV administration. Following SC administration of 20 mg mAb2, the dose-normalized exposure (AUC_{inf}/dose) was approximately 62% of the dose-normalized exposure following 10 mg IV administration.

TABLE 1C
Ratio of Central Values and 90% Confidence Intervals for AUC for
mAb2 Following Single Doses of 10 mg SC, 20 mg SC, and 10 mg IV

| | | | | | | 90% |
|---|---|---|---|---|---|---|
| | Test vs. | | Central Value | | Point | Confidence |
| Group | Reference | PK Parameter | Test | Reference | Estimate | Interval |
| mAb2 10 mg | 10 mg SC versus | AUCₜ | 5.69 | 30.4 | 0.1875 | 0.0880- |
| | 10 mg | (μg · day/mL) | | | | 0.3992 |
| SC | IV | AUC_{inf} | 23.9 | 32.4 | 0.5319 | 0.2289- |
| | | (μg · day/mL) | | | | 1.2359 |
| mAb2 20 mg | 20 mg SC | AUCₜ/Dose | 1.70 | 3.04 | 0.5792 | 0.4238- |
| | versus | (μg · day/mL/mg) | | | | 0.7916 |
| SC | 10 mg IV | AUC_{inf}/Dose | 2.15 | 3.24 | 0.6170 | 0.3801- |
| | | (μg · day/mL/mg) | | | | 1.0017 |

AUC = area under the concentration curve;
AUCₜ = AUC from time 0 to the last observable concentration;
AUC_{inf} = AUC from time 0 to infinity;
IV = intravenous;
SC = subcutaneous;
vs. = versus

### Immunogenicity Assessment

Assessment of immunogenicity (i.e., anti-drug antibody [ADA] formation) at Week 0 (Day 1) (pre-dose), Weeks 2, 4, 6, 8, 12, and 24 (Days 15, 29, 43, 57, 85, 169) was conducted, and 8/24 subjects were ADA positive across treatment Group 2 (10 mg SC) and Group 4 (10 mg IV). No subjects were ADA positive in treatment Group 3 (20 mg SC).

All subjects were ADA negative at baseline. Following administration of single SC or IV doses of mAb2, there was approximately 63% and 38% of treatment-emergent ADA across Group 2 (10 mg SC) and Group 4 (10 mg IV), respectively. There was no treatment-emergent ADA observed in Group 3 (20 mg SC) as shown in Table 1D below.

TABLE 1D
Incidence of ADA at Baseline and Treatment-Emergent ADA

| | Dose Group | | |
|---|---|---|---|
| | Group 2 mAb2 | Group 3 mAb2 | Group 4 mAb2 |
| | 10 mg SC | 20 mg SC | 10 mg IV |
| | (N = 8) | (N = 8) | (N = 8) |
| Baseline | 0% (0/8) | 0% (0/8) | 0% (0/8) |
| Incidence | | | |
| % (n/N) | | | |
| Treatment- | 63% (5/8) | 0% (0/8) | 38% (3/8) |
| Emergent ADA^{a} | | | |
| % (n/A) | | | |

^{a}Treatment-emergent ADA is defined when a subject was (1) ADA negative or missing assessment at baseline (prior to the first mAb2 dose) and became ADA positive at one or more time point post-baseline; or (2) ADA positive at baseline and showed a 4-fold or greater increase in titer values relative to baseline.

mAb2 exposures of subjects with positive ADA titers following a single dose of 10 mg SC trended lower than those with negative ADA titers, while exposures of subjects with positive ADA titers following a single dose of 10 mg IV were largely overlapping with or slightly higher than those with negative ADA titers as shown in FIG. 1E. One subject in the 10 mg SC group had all mAb2 concentrations below the LLOQ and was excluded from this analysis. This subject was ADA negative.

### Pharmacodynamic Results

In the 10 mg SC group, >95% receptor saturation on CD8+ T cells was observed for 14 days in 6/8 subjects and for 28 days in 1/8 subjects, while receptor saturation was incomplete in one subject, who had all mAb2 concentrations below the LLOQ (Table 1B). In the 20 mg SC group, >95% receptor saturation on CD8+ T cells was observed for 42 days in 5/8 subjects and for 28 days in 2/8 subjects, while receptor saturation was incomplete in one subject, who had all mAb2 concentrations below the LLOQ (Table 1B). In the 10 mg IV group, >95% receptor saturation on CD8+ T cells was observed for 56 days in 3/8 subjects, for 42 days in 1/8 subjects, for 28 days in 3/8 subjects and for 14 days in 1/8 subjects. The plots of mean (+SD) % free PD-1 receptor versus time are shown in FIG. 1F.

### Example 2 Study B

This Example described a study of multiple doses of the anti-PD-1 antibody mAb2 in HIV-1 infected adults.

### Study Objectives:

Objectives of this study included 1) evaluating the safety and tolerability of multiple doses of mAb2 (having heavy chains shown as SEQ ID NO:9 and may also have a C-terminal lysine truncated heavy chain of SEQ ID NO: 20 and light chains shown as SEQ ID NO:10) versus placebo in HIV-1 infected adult subjects and 2) evaluating the pharmacokinetics (PK) of multiple doses of mAb2 in HIV-1 infected adult subjects.

The exploratory objectives for this study were to evaluate peripheral receptor saturation (RS), the impact of mAb2 on the immune (including HIV-1-specific) response, the potential for mAb2 in latency reversal of the viral reservoir (cells harboring proviral DNA) (Stage I only), the impact of mAb2 on size of the viral reservoir, and the impact of mAb2 on viral load kinetics during ART interruption and ART restart.

### Background and Rationale

In this randomized, double-blind, placebo-controlled Phase 1b study, the safety, pharmacokinetics, and pharmacodynamics of multiple doses of mAb2 were evaluated in HIV-1 infected subjects without malignancy while virologically suppressed on ART and while experiencing viral rebound during an intensively-monitored treatment interruption of ART. The study was conducted in 2 stages; the safety of lower doses of mAb2 in Stage I was first established prior to evaluation of higher doses in Stage II.

### Investigational Plan

This was a Phase 1b, randomized, double-blind, placebo-controlled multiple-dose study of mAb2 in HIV-1 infected adults virologically suppressed on stable ART, with a planned intensively-monitored ART interruption.

This study was divided into two stages (Stage I and Stage II). The Stage I periods were as follows:
1. Study Drug Dosing Period (ART Suppressed); and
2. Post-Treatment Period (ART Interruption/ART Restart).

In the Study Drug Dosing Period for Stage I, subjects (n=25) were randomized in parallel (1:2:2) to receive either placebo (n=5), 2 mg mAb2 (n=10), or 10 mg mAb2 (n=10), respectively, intravenously (IV) once every 4 weeks (Q4W) for 2 doses at Week 0 (Day 1) and Week 4 (Day 29). Starting at Week 4 (Day 29) (following the second study drug dose), subjects entered the Post-Treatment Period and underwent an intensively-monitored ART interruption planned for 12 weeks, the final duration of which was based on ART restart criteria.

An assessment of the safety, PK, and PD data from Stage I was made prior to proceeding to Stage II, based on pre-specified criteria.

In Stage II, subjects (n=15) were randomized in parallel (1:2) to receive either placebo (n=5) or 10 mg mAb2 IV Q2W (n=10) for 4 doses at Week 0 (Day 1), Week 2 (Day 15), Week 4 (Day 29), and Week 6 (Day 43). Starting at Week 0 (Day 1) (following the first study drug dose), subjects underwent an intensively-monitored ART interruption planned for 12 weeks, the final duration of which was based on ART restart criteria.

For both Stages I and II, subject(s) who completed 12 weeks of ART interruption and did not meet ART restart criteria may have had an extended ART interruption up to a total of 36 weeks per the investigator's discretion and subject preference.

### Key Eligibility Criteria:

1. Subjects were male or female and in generally good health, 18 to 65 years of age, body mass index ≥18.0 to <35.0 kg/m².
2. HIV-1 infected subjects were on ART for at least 12 months prior to screening and on current ART regimen for at least 8 weeks prior to screening (regimen could not include a non-nucleoside reverse transcriptase inhibitor [NNRTI], maraviroc, or long-acting ART regimens).
3. Plasma HIV-1 RNA was below lower limit of quantification (LLOQ) at screening and at least 6 months prior to screening.
4. CD4+ T cell count was ≥500 cells/μL at screening and at least once during the 12 months prior to screening.
5. The subjects were willing to undergo ART interruption.
6. Subjects agreed to use an effective barrier method of protection (male and/or female condoms) during sexual activity for protection against HIV-1 transmission throughout the study.
7. Subjects had a CD4+ T cell nadir of ≥200 cells/μL during chronic infection.
8. Subjects did not have known resistance to ≥2 classes of ART.
9. Subjects did not have history of AIDS-defining illness.
10. Subjects did not have active or suspected malignancy or history of malignancy (other than basal cell skin cancer or cervical carcinoma in situ) in the past 5 years.
11. Subjects did not have history of or active immunodeficiency (other than HIV).
12. Subject did not have active autoimmune disease or history of autoimmune disease that has required systemic treatment.
13. Subject did not have prior receipt of immunomodulatory or immunosuppressive (including IV/PO steroids at any dose, but excluding steroids that are inhaled, topical or via local injection) therapy within 6 months prior to the first dose of study drug.
14. Subjects did not have prior therapy/exposure to mAb2 or any other immune checkpoint inhibitor (e.g., anti-PD-1, anti-PD-L1, anti-PD-L2, anti-CTLA-4).
15. Subjects did not have current hepatitis B virus or hepatitis C virus infection that might expose the subjects to undue risk of harm, confound study outcomes, or prevent the subject from completing the study (including but not limited to significant or unstable cardiac, neurologic or pulmonary disease, chronic active infectious disease except for HIV, chronic liver disease, poorly controlled diabetes mellitus and history of Stevens-Johnson Syndrome, toxic epidermal necrolysis (TEN), or drug reaction with eosinophilia and systemic symptoms (DRESS)).
16. Subjects did not have known psychiatric or substance abuse disorders that would interfere with adherence to study requirements.
17. Female subjects were not pregnant, breastfeeding, or considering becoming pregnant during the study.

### Treatment Duration:

In Stage I, subjects received two doses of mAb2 or placebo intravenously (IV) Q4W. In Stage II, subjects received four doses of mAb2 or placebo intravenously (IV) Q2W. The total study duration was approximately 32 to 42 weeks for most subjects.

### Evaluation

The study results were evaluated by specific endpoints described below.

AE, IRAE, and PK endpoints:

Study drug-related Grade 3 or higher adverse events (AEs), study drug related IRAEs, and pharmacokinetics were evaluated.

### Safety Endpoints:

Safety endpoints included frequency and severity of adverse events (AEs) and adverse events of special interest (AESIs), including immune related adverse events (IRAEs), infusion-related reactions, injection site reactions and hepatotoxicity. Safety evaluations included vital sign measurements, physical examinations, and clinical laboratory testing (including hematology and metabolic panels, thyroid function, hemolysis, plasma HIV-1 ribonucleic acid (RNA), and CD4+ T cell, B cell, and NK cell counts [TBNK panel]) throughout the study. Virologic failures were also evaluated.

### Pharmacokinetic Endpoints

The pharmacokinetic endpoints for mAb2 were the maximum observed plasma concentration (Cₘₐₓ) and time to Cₘₐₓ (Tₘₐₓ) following each dose of mAb2, observed concentration at the end of the dosing intervals (C_{trough}), area under the serum concentration-time curve over the dosing intervals (AUCₜₐᵤ), and terminal phase elimination half-life (t_{1/2}) following the last dose.

Immunogenicity of mAb2 was assessed by using a tiered approach for detecting anti-drug antibodies (ADA).

### Pharmacodynamic Endpoints

Blood samples were used to evaluate target engagement to inform PK and PD and to explore changes in biologic activities that may reflect the mechanism of action.
1. Peripheral PD-1 receptor saturation (%) on CD4+ and CD8+ T cell subsets by study drug. Blood samples were collected to determine PD-1 receptor expression and saturation by study drug on T cell subsets (e.g., naïve, central/effector/transient memory) by methods such as fluorescence activated cell sorting (FACS) analysis (markers including but not limited to CD3, CD4, CD8, CD28, CD95, PD-1)
2. Immunomodulatory effects pre- and post-study drug treatment;
   a. Induction of proliferation and activation markers;
   b. Serum cytokine/chemokines levels; and
   c. Ex vivo HIV-specific T cell response. Blood samples were collected to evaluate induction of proliferation and activation markers by FACS analysis (markers including but not limited to CD3, CD4, CD8, Ki67, OX40, CD25, HLA-DR on T cell subsets [e.g., naïve, central/effector/transient memory]). Serum samples were collected to evaluate changes in a panel of inflammation-related serum cytokine/chemokines markers (e.g., hs-CRP, D-Dimer, IL-6, CXCL9, CXCL10).
      Blood samples were collected for ex-vivo analysis of HIV-specific T cell functionality (e.g., cytokine response).
   d. Latency reversal (Stage I only):
      changes in HIV transcription and/or residual viremia in plasma pre- and post-study drug treatment.
   Blood samples were collected to measure latency reversal by assays including but not limited to quantitative polymerase chain reaction (PCR)-based evaluation of cell-associated HIV RNA and residual viremia (e.g., single copy RNA assay).
3. Size of HIV reservoir: changes in proviral DNA pre- and post-study drug treatment. Blood samples were collected to measure size of HIV reservoir using assays including, but not limited to, quantitative PCR-based evaluation of intact proviral DNA and total proviral DNA.
4. Viral load kinetics during ART Interruption:
   time to HIV-1 rebound to ≥1000 copies/mL during ART interruption;
   time to HIV-1 rebound to ≥200 copies/mL during ART interruption;
   meeting ART restart criteria (based on HIV-1 RNA, CD4+ T cell count, or other reasons [e.g., subject preference]) prior to Week 12 of ART interruption;
   HIV-1 RNA levels <200 copies/mL at 2, 4, 6, 8, and 12 weeks post-ART interruption; and
   HIV-1 RNA levels <50 copies/mL at 2, 4, 6, 8, and 12 weeks post-ART interruption.
5. Time to viral re-suppression after ART restart.

### Results

Assessed endpoints included: frequency and severity of adverse events, adverse events of special interest (AESIs) such as IRAEs, retroviral rebound syndrome, infusion-related reactions, and hepatotoxicity.

The adverse events observed in the study are shown in Table 2A. No deaths, no serious adverse events (SAEs) and no adverse events AEs ≥Grade 3 related to study drug were reported. There were no infusion-related adverse events and no hepatic-related adverse events reported in the study.

Two subjects receiving mAb2 experienced IRAEs (one each), both assessed as having a reasonable possibility of being related to study drug. One case of thyroiditis (Grade 1, Day 59) occurred in the mAb2 10 mg IV group of Stage 2 and resolved within 84 days without intervention. One case of hyperthyroidism (Grade 1, Day 3) occurred in the mAb2 10 mg IV group of Stage 2 that resolved within 69 days without intervention; in this case, the study drug was withdrawn due to the adverse event.

One case of (Retroviral Rebound Syndrome) RRS (Grade I, estimated onset Day 29) occurred in the mAb2 10 mg IV group of Stage 2 that resolved with ART restart.

TABLE 2A
mAb2 in PLWH: Overview of Subjects with Treatment
Emergent Adverse Events (AEs) in Study B.

| | Study B Stage 1 (MAD Q4W) | | | Study B Stage 2 (MAD Q2W) | |
|---|---|---|---|---|---|
| | | 10 mg | | 10 mg | |
| | 2 mg IV | IV Q4W | Placebo | IV Q2W | Placebo |
| n (% of patients) | (n = 10) | (n = 10) | (n = 5) | (n = 11) | (n = 5) |
| Any AE | 6 (60%) | 6 (60%) | 2 (40%) | 11 (100%) | 4 (80%) |
| Drug-related AE | 2 (20%) | 2 (20%) | 0 | 5 (45.5%) | 3 (60%) |
| SAE | 0 | 0 | 1 (20%) | 0 | 0 |
| Drug-related SAE | 0 | 0 | 0 | 0 | 0 |
| AE leading to | 0 | 0 | 0 | 1 (9.1%) | 0 |
| discontinuation of SD | | | | | |
| AE grade 3 or higher | 1 (10%) | 0 | 1 (20%) | 0 | 0 |
| Hepatic-related AESI | 0 | 0 | 0 | 0 | 0 |
| IRAEs (any grade) | 0 | 1 (10%) | 0 | 1 (9.1%) | 0 |
| AE corresponding to | 0 | 0 | 0 | 1 (9.1%) | 0 |
| RRS | | | | | |

No subject met the Department of Health and Human Services (DHHS) Guidelines defined virological failure criteria and no change from baseline ART regimen due to the presence of resistant virus was needed in the study for any subject. This study evaluated mAb2 multiple doses that were significantly lower than the doses used for oncologic indications in order to enhance tolerability and safety profile in PLWH which is a new population. Overall, no new safety signals associated with the use of mAb2 in the HIV-1 subject population were detected.

### Laboratory Summary

There were no clinically significant trends observed for changes in CD4 T cell count or hematology, chemistry, or urinalysis parameters. One subject that received placebo had a Grade 2 CD4 count decline during Analytical Treatment Interruption (ATI) period at Day 50. CD4 was normalized above baseline values on Day 64 after ART restart. No AE was reported related to this CD4 count decline.

No subject that received the active drug met criteria for potentially clinically significant values for hemoglobin with Grade ≥2 and no trends on hematocrit and or hemoglobin decline were observed. There were no AEs related to hemolysis or anemia reported in the study.

Potentially clinically significant Grade 2 creatinine elevation was observed in non-sustained single timepoints in 4 subjects. None were considered clinically significant as an AE by the PI.

### Vital Signs Summary

There was no clinically significant trend observed during the infusion monitoring period or in the study overall for any vital sign parameter. There were no vital signs-related AEs reported.

### ECG Summary

ECGs were evaluated for entry into the study at baseline and could be performed as needed for any post-baseline safety evaluation. No subject necessitated a post-baseline ECG evaluation. There were no ECG-related AEs reported. One subject reported an AE of tachycardia during the study. The event was mild and considered by the investigator as not related to the study drug.

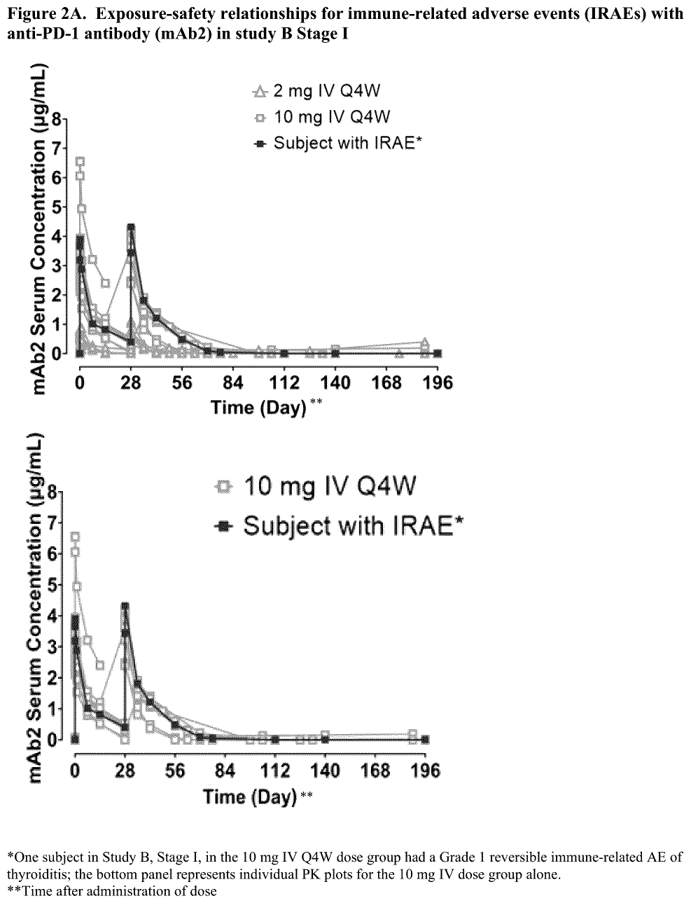

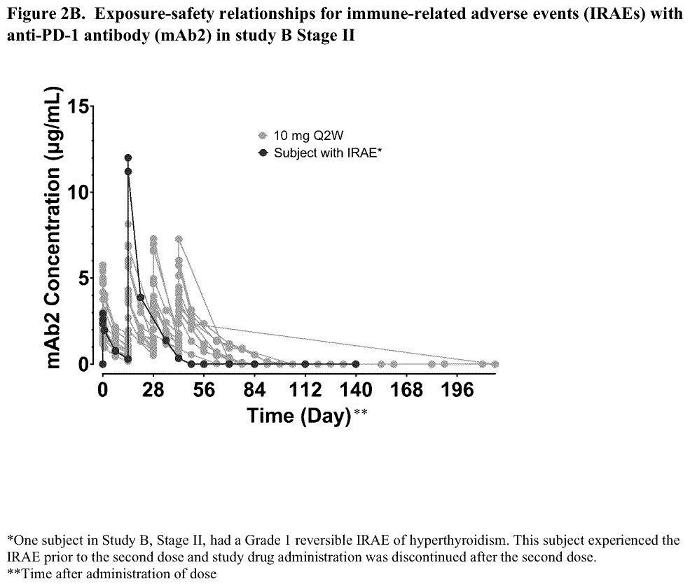

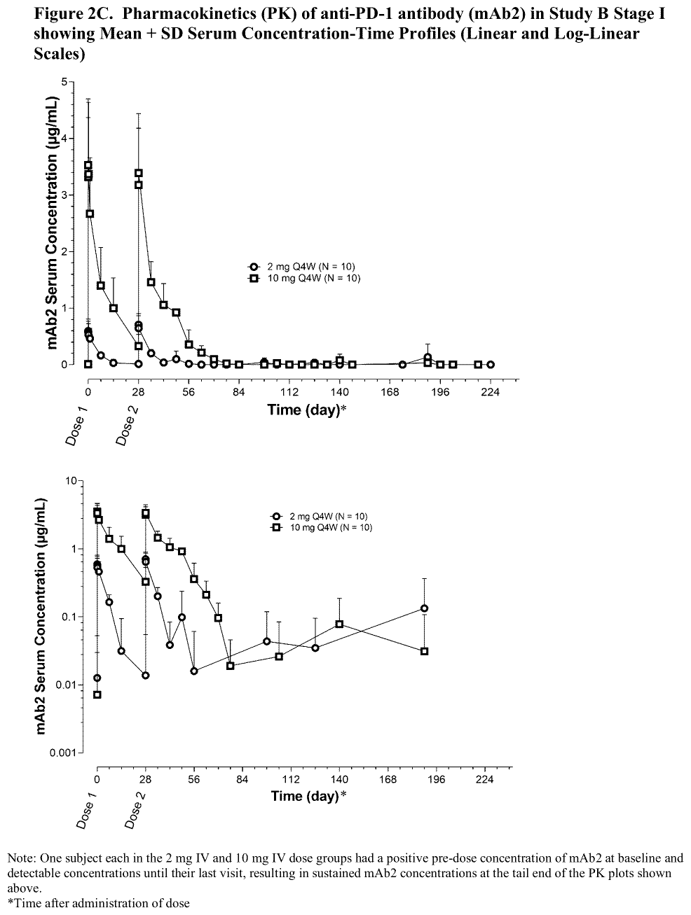

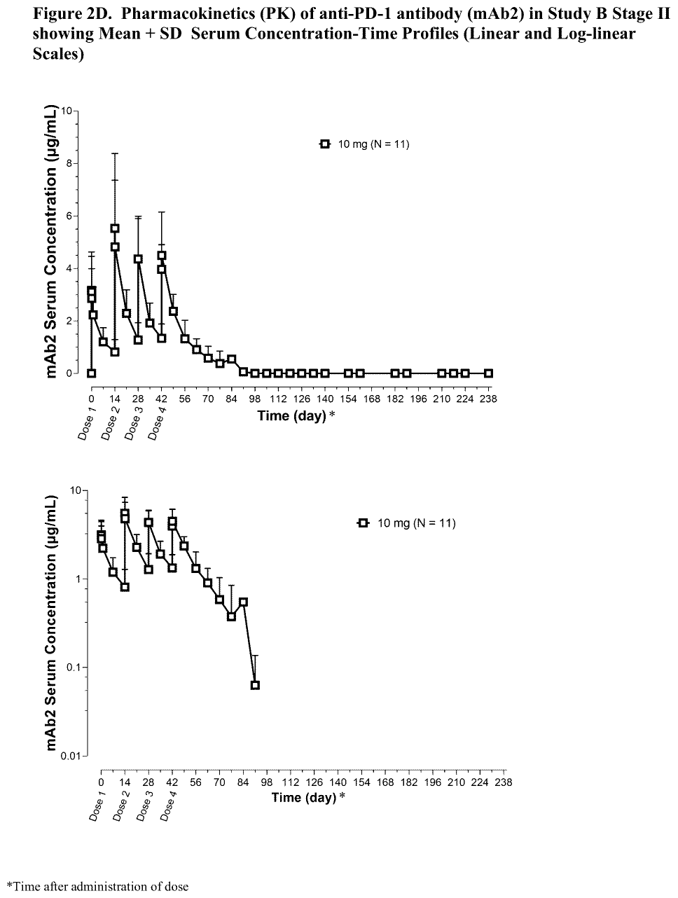

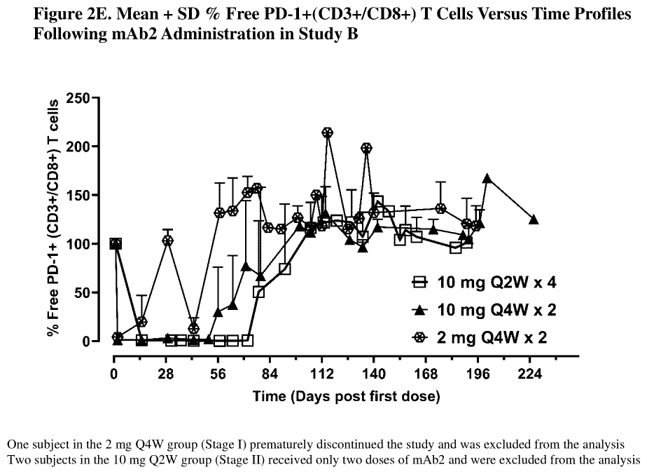

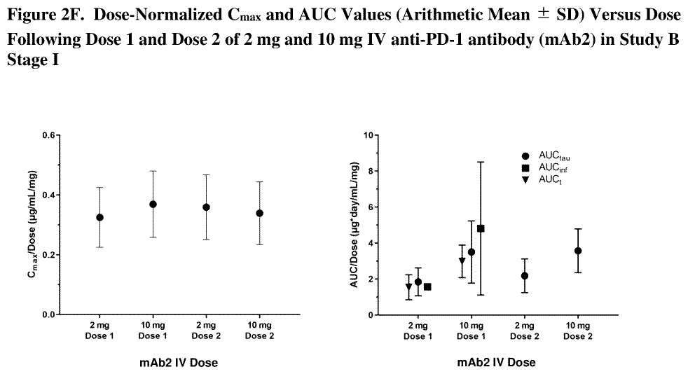

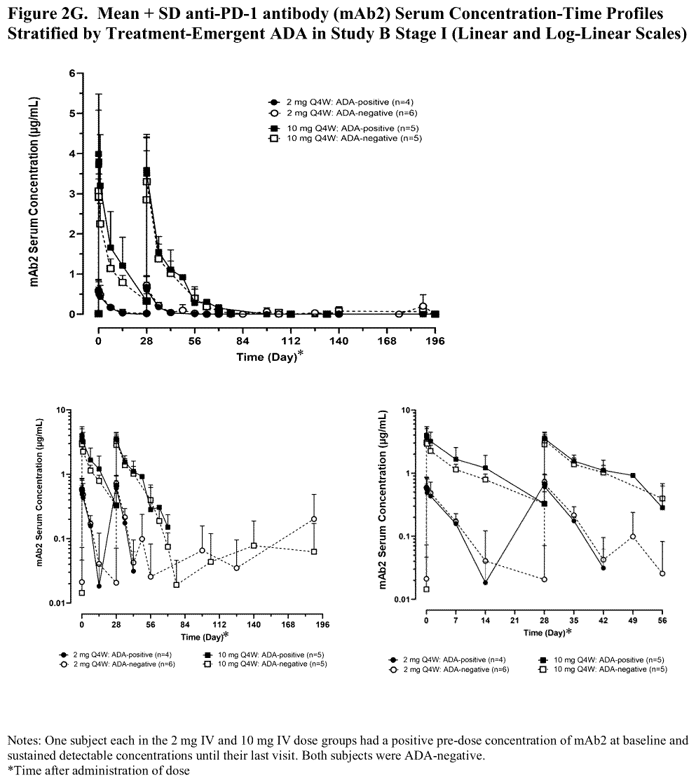

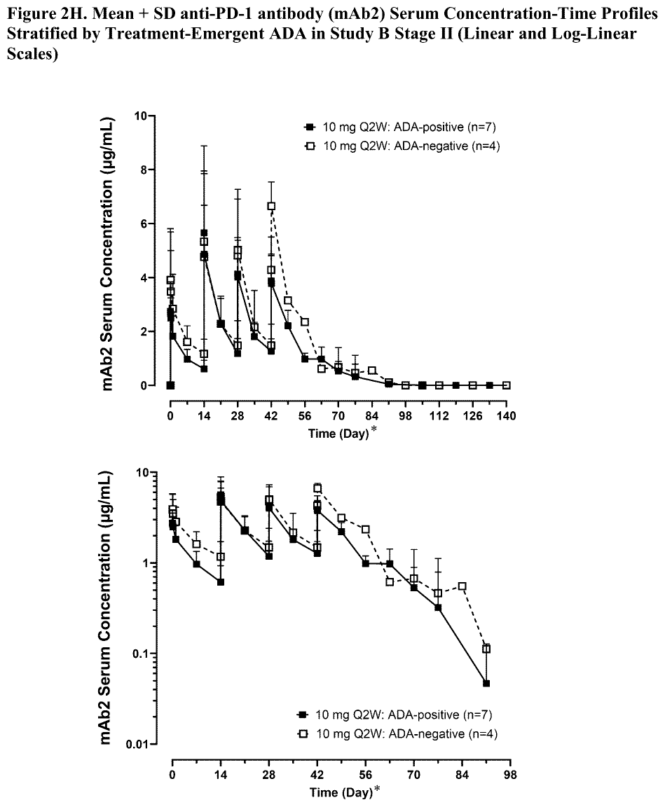

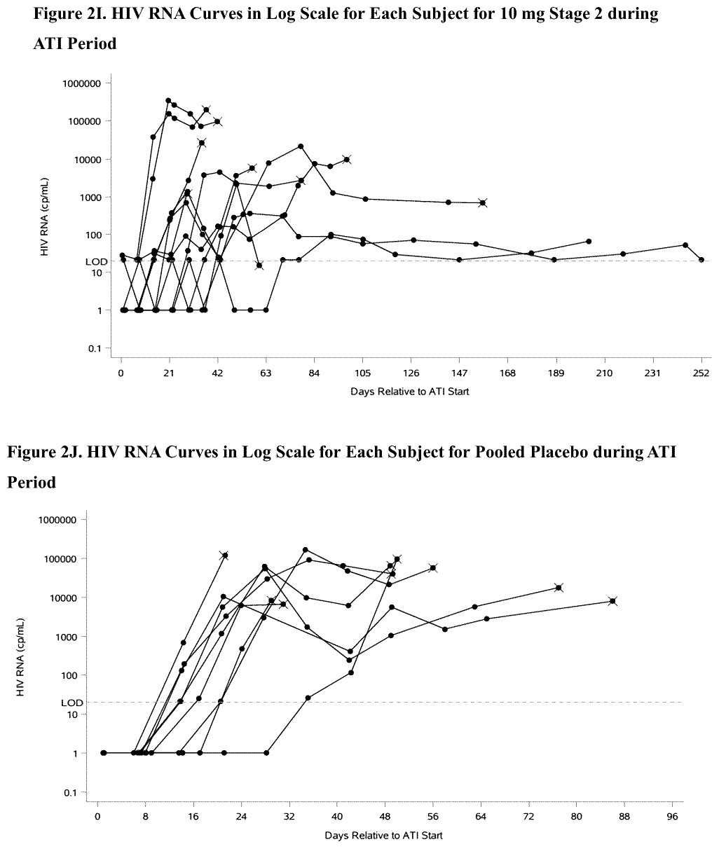

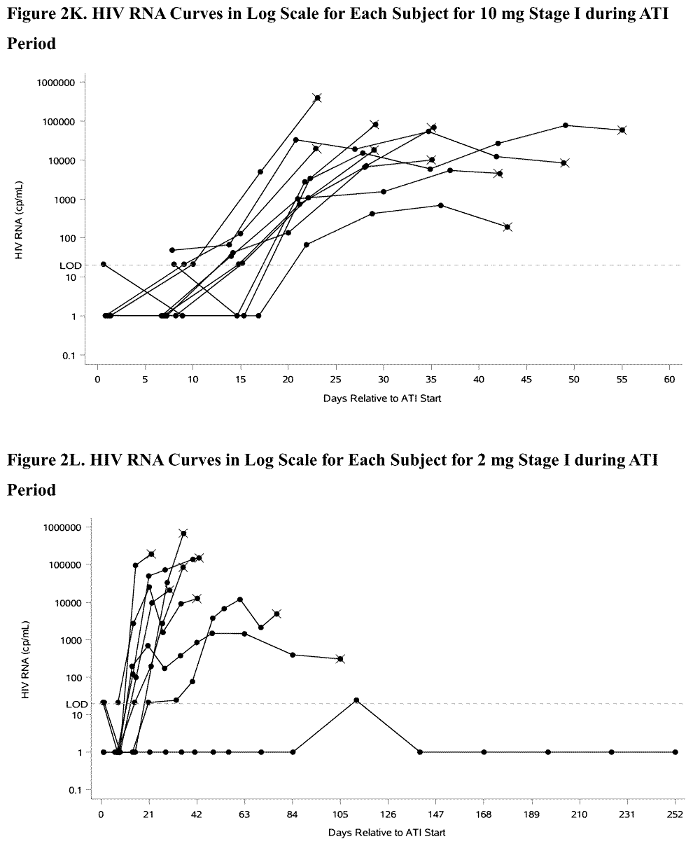

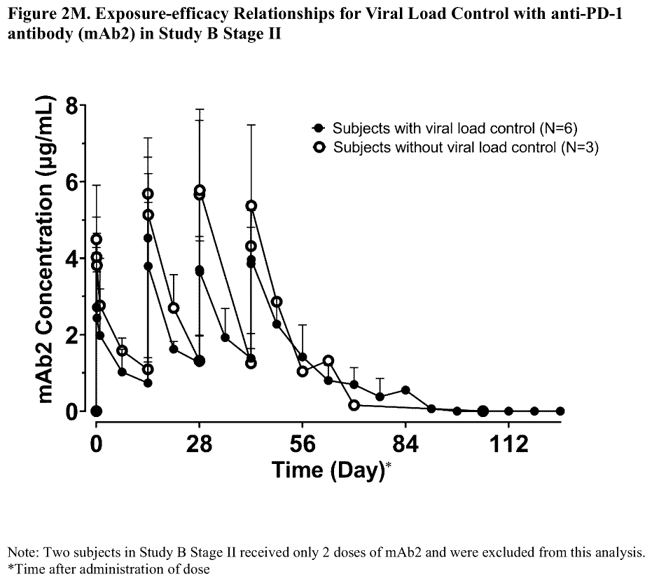

Assessment of mAb2 exposure-response relationships for safety (IRAEs): Potential exposure-response relationships for safety in Study B (Stages I and II) were evaluated by comparing the exposures of subjects who demonstrated safety signals (IRAEs) with those who did not. Individual plots of mAb2 PK were stratified by safety (IRAEs) and are depicted in FIGS. 2A and 2B. No apparent trends or exposure-response relationships were identified. The exposures of subjects with safety signals for IRAEs across Study B were comparable with the exposures of the remaining subjects in their respective dose groups.

PK of mAb2 in Study B

The PK profiles for mAb2 in Study B (Stages I and II) are presented in FIGS. 2C and 2D.

The PK parameters of mAb2 in Study B (Stages I and II) are presented in Tables 2B and 2C.

TABLE 2B
Geometric Mean (Mean, % CV) Pharmacokinetic Parameters of mAb2 from Study B Stage I.

| | PK Parameters Following Dose 1 | | | | PK Parameters Following Dose 2 | | | |
|---|---|---|---|---|---|---|---|---|
| | (Week 0 (Day 1)) | | | | (Week 4 (Day 29)) | | | |
| Pharmacokinetic | 2 mg IV | | 10 mg IV | | 2 mg IV | | 10 mg IV | |
| Parameter (unit) | (N = 10) | | (N = 10) | | (N = 9)^{a} | | (N = 9)^{b} | |
| Cₘₐₓ (μg/mL) | 0.602 | (0.65, 31) | 3.57 | (3.69, 30) | 0.684 | (0.719, 30)^{f} | 3.2 | (3.39, 31)^{f} |
| Tₘₐₓ^{c} (h) | 3.4 | (1.5-167) | 2.6 | (1.3-5.5) | 1.6 | (1.3-3.1)^{f} | 1.3 | (1.1-191)^{f} |
| C_{trough} (μg/mL) | 0.124 | (0.0138, 300)^{f} | 0.401 | (0.479, 100)^{f} | 0.128 | (0.016, 283)ⱼ | 0.342 | (0.359, 72)^{g} |
| AUCₜₐᵤ | 3.48 | (3.68, 43)^{g} | 32 | (35, 49) | 4.07 | (4.37, 43)^{j} | 33.5 | (35.7, 34)^{f} |
| (μg · day/mL) | | | | | | | | |
| AUC_{inf} | 3.13 | (3.13, 8)^{h} | 39.7 | (48.1, 77)ⁱ | — | | — | |
| (μg · day/mL) | | | | | | | | |
| Cₘₐₓ/dose | 0.301 | (0.325, 31) | 0.357 | (0.369, 30) | 0.342 | (0.359, 30)^{f} | 0.32 | (0.339, 31)^{f} |
| (μg/mL/mg) | | | | | | | | |
| AUCₜₐᵤ/dose | 1.74 | (1.84, 43)^{g} | 3.2 | (3.5, 49) | 2.04 | (2.18, 43)^{j} | 3.35 | (3.57, 34)^{f} |
| (μg · day/mL/mg) | | | | | | | | |
| AUC_{inf}/dose | 1.56 | (1.57, 8)^{h} | 3.97 | (4.81, 77)ⁱ | — | | — | |
| (μg · day/mL/mg) | | | | | | | | |

| | | | | | |
|---|---|---|---|---|---|
| t_{1/2}^{d} (day) | — | — | 3.24, 4.62^{e} | 5.57 | (1.09)^{k} |

AUC = area under the concentration-time curve; AUC_{inf} = AUC from time 0 to infinity; AUCₜₐᵤ = area under the concentration-time curve over the dosing interval; Cₘₐₓ = maximum observed concentration; C_{trough} = observed concentration at the end of the dosing interval; CV = coefficient of variation; IV = intravenous; PK = pharmacokinetic; t_{1/2} = terminal elimination half-life; Tₘₐₓ = time to maximum observed concentration; — = not calculated.
^{a}One participant prematurely discontinued prior to the second dose and was not included in the Dose 2 summary.
^{b}One participant was excluded from the Dose 2 summary as they had a dosing interval error (i.e., Dose 2 was delayed by 3 weeks)
^{c}Tₘₐₓ presented as median (minimum-maximum)
^{d}t_{1/2} presented as harmonic mean and pseudo standard deviation
^{e}N ≤ 2 are presented as individual values
^{f}N = 9
^{g}N = 6
^{h}N = 4
ⁱN = 5
^{j}N = 8
^{k}N = 7

TABLE 2C
Geometric Mean (Mean, % CV) Pharmacokinetic Parameters of mAb2 from Study B Stage II.

| | PK Parameters for 10 mg IV Q2W (N = 11)^{a} | | | | | | | |
|---|---|---|---|---|---|---|---|---|
| | Dose 1 | | Dose 2 | | Dose 3 | | Dose 4 | |
| Pharmacokinetic | (Week 0 | | (Week 2 | | (Week 4 | | (Week 6 | |
| Parameter (unit) | (Day 1)) | | (Day 15)) | | (Day 29)) | | (Day 43)) | |
| Cₘₐₓ (μg/mL) | 3.1 | (3.35, 43) | 4.62 | (5.23, 55) | 4.26 | (4.49, 36)^{e} | 4.35 | (4.55, 33)^{e} |
| Tₘₐₓ^{b} (h) | 2.4 | (0.6-4.8) | 0.7 | (0.6-2.6) | 2.3 | (0.6-2.6)^{e} | 1.5 | (0.6-2.6)^{e} |
| C_{trough} (μg/mL) | 0.68 | (0.814, 58) | 1.26 | (1.40, 46) | 1.24 | (1.34, 41)^{e} | 1.21 | (1.32, 53)^{f} |
| AUCₜₐᵤ | 18 | (19.9, 48) | 33.8 | (36.8, 46)^{d} | 27.1 | (27.7, 25)^{f} | 36.7 | (39.5, 44)^{e} |
| (μg · day/mL) | | | | | | | | |
| Cₘₐₓ/dose | 0.31 | (0.335, 43) | 0.462 | (0.523, 55) | 0.426 | (0.449, 36)^{e} | 0.435 | (0.455, 33)^{e} |
| (μg/mL/mg) | | | | | | | | |
| AUCₜₐᵤ/dose | 1.8 | (1.99, 48) | 3.38 | (3.68, 46)^{d} | 2.71 | (2.77, 25)^{f} | 3.67 | (3.95, 44)^{e} |
| (μg · day/mL/mg) | | | | | | | | |

| | | | |
|---|---|---|---|
| t_{1/2}^{c} (day) | — | 7.99 | (3.42)^{g} |

AUC = area under the concentration curve; AUC_{inf} = AUC from time 0 to infinity; AUCₜₐᵤ = area under the concentration-time curve over the dosing interval; Cₘₐₓ = maximum observed concentration; C_{trough} = observed concentration at the end of the dosing interval; CV = coefficient of variation; IV = intravenous; PK = pharmacokinetic; t_{1/2} = terminal elimination half-life; Tₘₐₓ = time to maximum observed concentration; — = not calculated.
^{a}Two participants who only received the first two doses were excluded from the summary for Doses 3 and 4
^{b}Tₘₐₓ presented as median (minimum-maximum)
^{c}t_{1/2} presented as harmonic mean and pseudo standard deviation
^{d}N = 10
^{e}N = 9
^{f}N = 4
^{g}N = 6

Following administration of 2 mg IV and 10 mg IV doses of mAb2 in Study B Stage I, the geometric mean Cₘₐₓ was 0.602 and 3.57 μg/mL after the first dose, and 0.684 and 3.2 μg/mL after the second dose, respectively. The maximum mAb2 serum concentrations were observed at approximately 1 to 3 hours from the start of infusion. The terminal elimination half-life was approximately 3-5 days and 6 days for the 2 mg IV and 10 mg IV regimens, respectively.

In Study B Stage II, the Cₘₐₓ after the first and last (4^{th}) dose of mAb2 was 3.1 and 4.35 g/mL and time to maximum observed serum concentration was at a median of approximately 1 to 2 hours from the start of infusion. The terminal elimination half-life was approximately 8 days.

### PD-1 Receptor Saturation

Baseline expression of PD-1 on CD4+ and CD8+ T cells and subsets were comparable across dose groups. The expression of PD-1 was higher on central/transient memory (CD28+/CD95+) as compared to naïve cells (CD28+/CD95−). The duration of PD-1 receptor saturation was comparable across CD4+ and CD8+ T cells and memory subsets. The duration of 95% PD-1 receptor saturation by mAb2 on CD8+ T cells after the last dose is shown in Table 2D.

TABLE 2D
Median (Mean, SD) Duration of >95% PD-1 Receptor
Saturation on CD8+ T cells

| | Stage I | Stage I | Stage II |
|---|---|---|---|
| | 2 mg IV | 10 mg IV | 10 mg IV |
| | (N = 3) | (N = 9) | (N = 10) |
| Duration | 15.0 (10.7, 7.51) | 29.0 (28.6, 13.9) | 29.5 (32.3, 11.0) |
| (day) | | | |
| of >95% | | | |
| PD-1 RS | | | |

In the 2 mg IV group in Stage I, >95% receptor saturation on CD8+ T cells was observed for <15 days. In the 10 mg IV group in Stages I and II, >95% receptor saturation on CD8+ T cells was observed for a median 29 days and 30 days, respectively. Hence, with 10 mg Q2W×4 doses, the total period of >95% receptor saturation was around 70 days. The plot of mean (+SD) % free PD-1 receptor versus time is shown in FIG. 2E. For the 10 mg Q2W (×4 doses) regimen, PD-1 saturation returned to baseline levels (˜100% free receptor), consistent with decline in mAb2 concentrations to sub-saturating levels (<0.1 μg/mL), by approximately Week 14 (Day 98) post-ATI.

Dose-Proportionality Assessment of mAb2 in Study B Stage I

Dose proportionality of mAb2 between the 2 mg IV and 10 mg IV doses in Stage I was investigated using an ANCOVA of the log-transformed dose-normalized Cₘₐₓ and AUC values. There was a significant difference in mAb2 dose-normalized AUC values (AUCₜₐᵤ, AUCₜ, and AUC_{inf} for Dose 1; AUCₜₐᵤ for Dose 2) between the 2 mg IV and 10 mg IV doses. The results of the ANCOVA of dose-normalized AUC values suggest that exposures following 10 mg IV dosing were more than dose-proportional relative to exposure following 2 mg IV dosing, indicating target-mediated drug disposition at the 2 mg IV dose level. FIG. 2F depicts the dose-normalized Cₘₐₓ and AUC values following Dose 1 and Dose 2 of 2 mg and 10 mg IV mAb2 in Stage I of Study B.

Dose proportionality was also assessed between the preliminary exposures of the doses evaluated in Study B with those observed at the higher oncologic doses of up to 500 mg IV in the mAb2 development program for oncology. Exposures between 10 mg IV mAb2 and the higher doses administered in cancer patients in the oncology development program were approximately dose-proportional based on AUC overdosing intervals of 4 weeks and 2 weeks. However, exposures for the 2 mg IV mAb2 dose were less than dose-proportional based on AUC calculations, indicating PK non-linearity at this dose level, characteristic of target-mediated drug disposition.

Accumulation Assessment of mAb2

Repeated measures and analyses were performed to assess accumulation of mAb2 after administration of mAb2 on Week 0 (Day 1) (Dose 1) versus Week 4 (Day 29) (Dose 2) in Stage I and Week 0 (Day 1) (Dose 1) versus Week 6 (Day 43) (Dose 4) in Stage II. The ratio of the central values (estimate) and the 90% confidence intervals for the first dose versus the last dose accumulation tests of the natural logarithm of Cₘₐₓ and AUCₜₐᵤ for mAb2 are presented in Table 2E.

TABLE 2E
Analysis of Repeated Measures and 90% Confidence Intervals to
Assess Accumulation Ratio Following the Last Dose Versus the First Dose
for the Natural Logarithm of Cₘₐₓ and AUCₜₐᵤ for mAb2

| | | | | 90% |
|---|---|---|---|---|
| | | Pharmacokinetic | Point | Confidence |
| Treatment | Test vs. Reference | Parameter (unit) | Estimate | Interval |
| Stage I: | Dose 2 (Week 4 (Day 29)) | Cₘₐₓ (μg/mL) | 1.15 | 0.989-1.337 |
| 2 mg IV Q4W | vs. Dose 1 (Week 0 (Day | AUCₜₐᵤ (μg · day/mL) | 1.331 | 0.962-1.841 |
| | 1)) | | | |
| Stage I: | Dose 2 (Week 4 (Day 29)) | Cₘₐₓ (μg/mL) | 0.961 | 0.791-1.169 |
| 10 mg IV Q4W | vs. Dose 1 (Week 0 (Day | AUCₜₐᵤ (μg · day/mL) | 1.155 | 1.019-1.308 |
| | 1)) | | | |
| Stage II: | Dose 4 (Week 6 (Day 43)) | Cₘₐₓ (μg/mL) | 1.518 | 1.236-1.864 |
| 10 mg IV Q2W | vs. Dose 1 (Week 0 (Day | AUCₜₐᵤ (μg · day/mL) | 2.155 | 1.766-2.63 |
| | 1)) | | | |

The results indicate negligible accumulation of mAb2 with a Q4W dosing interval at the 2 mg IV and 10 mg IV dose levels in Stage I, based on Cₘₐₓ and AUCₜₐᵤ following Dose 2 and Dose 1. Given that subjects were suppressed on ART during the first 4 weeks in Stage I of the study and underwent ATI after Week 4, the consistency in exposures during both periods indicates no apparent impact of ART status or viremia on mAb2 exposures over the dosing interval of 4 weeks. In Stage II of the study, accumulation of approximately 1.5-fold and 2-fold was observed based on Cₘₐₓ and AUCₜₐᵤ, respectively, following Dose 4 as compared to Dose 1, suggesting drug accumulation with a Q2W dosing interval at the 10 mg IV dose level of mAb2.

### Immunogenicity Assessment

Assessment of immunogenicity (i.e., ADA formation) was conducted at Week 0 (Day 1) (pre-dose), and throughout the treatment, post-treatment, and viral rebound periods in Study B. The treatment-emergent ADA incidence for each dosing regimen of mAb2 evaluated in PLWH in Study B is summarized in Table 2F below.

TABLE 2F
Summary of ADA Incidence Following Multiple
Doses of mAb2 in Study B Stages I and II

| | Dose Group | | |
|---|---|---|---|
| | Stage I | | Stage II |
| | 2 mg IV Q4W | 10 mg IV Q4W | 10 mg IV Q2W |
| Baseline | 0% (0/10) | 0% (0/10) | 0% (0/11) |
| Incidence | | | |
| % (n/N) | | | |
| Treatment- | 40% (4/10) | 50% (5/10) | 64% (7/11) |
| Emergent ADA^{a} | | | |
| % (n/A) | | | |

^{a}Treatment-emergent ADA is defined when a subject was (1) ADA negative or missing assessment at baseline (prior to the first mAb2 dose) and became ADA positive at one or more time point post-baseline; or (2) ADA-positive at baseline and showed a 4-fold or greater increase in titer values relative to baseline.

Sixteen out of thirty-one (16/31) subjects were ADA-positive across all mAb2 treatment arms in the study. All subjects were ADA-negative at baseline. Following administration of IV doses of mAb2, there was approximately 40%, 50%, and 64% of treatment-emergent ADA in the 2 mg IV and 10 mg IV treatment arms in Stage I and the 10 mg IV treatment arm in Stage II, respectively.

mAb2 concentration-time profiles stratified by treatment-emergent ADA status (ADA-positive or -negative) in Stages I and II of the study are depicted in FIGS. 2G and 2H. Overall, the exposures of subjects who were ADA-positive largely overlapped with those who were ADA-negative across all treatment regimens in Stages I and II, suggesting no apparent impact of ADA status on the PK of mAb2.

Suppression of Viral Load Following Treatment with mAb2

HIV-1 viral load was monitored in all participants following ART interruption. ART restart was determined using protocol-defined criteria; participants could be restarted anytime if requested. Delay in viral rebound and/or viral control was observed following completed IV treatment with mAb2 from Study B in subjects that undergone ATI (FIG. 2I).

In those individuals who received two doses Q4W of mAb2 in stage I, no viral control post viral rebound was observed. This was similar to the placebo group (FIGS. 2J to 2L). A single subject from Stage I (2 mg IV Q4W) did not experience rebound and maintained a VL <50 copies/mL off ART (by subject and PI preference) through the end of the study (FIG. 2L).

Also, median time to viral rebound to ≥1000 copies/mL was 36 days in the Stage 11 10 mg IV group (N=11) compared to 21 days in the combined Stage I and Stage II placebo groups (N=10). In contrast, of 11 participants starting Q2W×4 doses, 9 completed their doses and experienced viral rebound (>200 cp/mL). Of these, however, 6 participants experienced delayed viral rebound (>21 days; placebo median) and/or off-ART viral control (<1000 cp/mL) post-rebound, with 4 not meeting ART-restart criteria and 2 re-starting ART based on personal request. Of the 4 not meeting ART-restart criteria, 2 opted to continue ATI until the end of the study and maintained viral control of <200 cp/mL through 29-36 weeks post-ATI initiation (FIG. 2I).

Exposure-response relationships for efficacy (viral load (VL) control) in Study B (Stage II only) were evaluated by comparing the exposures of subjects who demonstrated efficacy signals (viral load control) with those who did not. Individual plots of mAb2 PK stratified by efficacy are depicted in FIG. 2M. No apparent trends or exposure-response relationships were identified. For subjects who received all 4 doses of mAb2 10 mg IV in Study B Stage II, the exposures of subjects who demonstrated efficacy signals of viral load control were comparable to the exposures of those who did not have viral load control.

### Example 3 Study C

A randomized, double-blind, placebo-controlled, Phase 1a study was conducted to evaluate the safety, pharmacokinetics, and pharmacodynamics of single ascending doses of mAb1 (anti-α4β7 integrin monoclonal antibody) in healthy adult volunteers.

### Study Objectives:

The main objectives of this study were to evaluate the safety, tolerability, pharmacokinetics, and immunogenicity of single-ascending doses of mAb1 (having heavy chains shown as SEQ ID NO:18 and may also have a C-terminal lysine truncated heavy chain of SEQ ID NO: 21 and light chains shown as SEQ ID NO:19) in healthy adult volunteers.

### Background and Rationale

mAb1 demonstrated a favorable safety profile in vitro and in preclinical studies. Evaluation of safety, tolerability, pharmacokinetics, and immunogenicity of single-ascending doses (SADs) of mAb1 in healthy adult human volunteers allowed for the selection of dose(s) to further evaluate in Phase 1b and 2.

### Investigational Plan

This first-in-human, double-blind, randomized, placebo-controlled, single-ascending dose (SAD) study was conducted in 7 groups in healthy adult volunteers (Groups 1, 2, 2a, 3, 4, 5, and 6). Groups 1, 2, 3, 4, and 6 had 8 subjects in each group, wherein 6 subjects received mAb1 and 2 subjects received placebo via intravenous (IV) infusion. Group 5 had planned to have up to 8 subjects enrolled. Six subjects were actually enrolled, wherein 5 subjects received mAb1 and 1 subject received placebo via IV infusion. Group 2a had 9 subjects, wherein 6 subjects received mAb1 and 3 subjects received placebo via subcutaneous (SC) injection.

### Study Groups:

Group 1: mAb1 200 mg IV single dose or placebo
Group 2: mAb1 400 mg IV single dose or placebo
Group 2a: mAb1 800 mg SC single dose or placebo (dosing commenced after group 2 confinement has ended)
Group 3: mAb1 800 mg IV single dose or placebo
Group 4: mAb1 1200 mg IV single dose or placebo
Group 5: mAb1 1800 mg IV single dose or placebo
Group 6: mAb1 50 mg IV single dose or placebo

Approximately 55 adult male and female volunteers in general good health were enrolled in this study.

### Discussion of Study Design and Choice of Control Groups

A double-blinded, placebo-controlled study design was used and was generally acknowledged as the standard for unbiased estimates of treatment (mAb1 versus matching placebo) differences. The dosing schedule was designed such that the successively higher doses were administered only after the review of at least 2 weeks (through Week 2 (Day 15)) of safety data of subjects from the preceding dose group (within the same route of administration) in conjunction with available pharmacokinetic, safety, and tolerability data from beyond 2 weeks in previously dosed group(s).

### Dose Escalation and Pausing Criteria

The decision to escalate to a new dosing group was determined after review of all available safety data from the preceding group(s) within the same route of administration (Groups 2, 3, 4, and 5 for IV infusion and Group 2a for SC injection).

Dose escalation for Groups 2 through Group 5 took place for the subsequent dose group after at least 2 weeks (through Week 2 (Day 15)) of safety data of all subjects from the preceding dose group were evaluated. Planned dose levels were changed after review of pharmacokinetic, safety and tolerability data from all subjects from the preceding group(s) in conjunction with available data from beyond 2 weeks in previously dosed group(s). Dose escalation evaluation of 2 weeks of safety data were not used to enroll groups with lower dose levels.

For all groups, if one of the following dose cohort level criteria was met, and the subject was confirmed to have been receiving mAb1, dosing for all subjects in that dose group or a higher dose group were paused until a safety evaluation of the event(s) was completed; dosing and enrollment of lower dose group(s) were continued as long as pause criteria for these group(s) have not been met.

### Clinical and Post Dose Systemic Adverse Events

1. Any subject administered active study drug experienced a severe (Grade 3) or higher clinical adverse event, considered associated to study drug.
2. Two or more subjects administered active study drug experienced a Grade 2 clinical adverse event of a similar nature, considered related to study drug.
3. A subject who received active study drug experienced a ≥Grade 3 injection site reaction, considered related to study drug.
4. Two or more subjects administered active study drug experienced Grade 2 suspected hypersensitivity reactions, or one subject administered active study drug experiences ≥Grade 3 suspected hypersensitivity reaction. Events were graded by the level of intervention described in the National Institutes of Health (NIH) Division of AIDS (DAIDS) Table for Grading the Severity of Adult and Pediatric Adverse Events ([DAIDS][v2.1][July 2017]), allergic reaction/cytokine release syndrome adverse event categories or, if applicable, anaphylaxis or serum sickness adverse event categories.

### Laboratory or Vital Sign Abnormalities, ECG Changes

1. Two or more subjects administered active study drug experienced a confirmed Grade 3 or higher hematologic laboratory abnormality related adverse event of similar nature considered related to study drug.
2. Two or more subjects administered active study drug experienced a confirmed Grade 2 or higher non-hematologic laboratory or vital sign abnormality related adverse event of similar nature considered related to study drug.
3. Any subject administered active study drug having confirmed ALT or AST >3×upper limit of normal (ULN) and total bilirubin >2×ULN or international normalized ratio (INR) >1.5, for which no alternative etiology was identified.
4. Any subject administered active study drug having confirmed ALT or AST >3×ULN with the appearance of fatigue, nausea, vomiting, right upper quadrant pain or tenderness, fever, rash, and/or eosinophilia (>5%), for which no alternative etiology was identified.
5. Any subject administered active study drug having a confirmed ALT or AST >8×ULN, for which no alternative etiology was identified.
6. Any subject administered active study drug having confirmed ECG changes that were considered clinically significant and considered related to study drug, a confirmed absolute QTcF value >500 msec for which the investigator considered related to study drug, or a confirmed absolute QTcF value change from baseline >60 msec.

### Appropriateness of Measurements

Standard pharmacokinetic, clinical, and laboratory procedures were utilized in this study.

### Suitability of Subject Population

The selection of subjects in general good health was standard for pharmacokinetic studies, promotes compliance, and reduced variability. Healthy volunteers were considered appropriate for this first-in-human study based on the following:

The selection of a relatively homogeneous population presented no confounding effects of advancing age, concomitant disease, or concomitant therapy.

Lower probability of prior exposure to an antibody-based immunotherapy was sought.

Subject risk was mitigated by the protocol-defined eligibility criteria, study design features, and specified safety monitoring procedures.

### Selection of Doses in the Study

The dose selection in this study was based on the collective data from preclinical toxicology, pharmacokinetic, and pharmacodynamic studies in cynomolgus monkeys.

Predicted safety margins for the starting and the top dose levels, as well as the lowest dose level, are summarized in Table 3A. The safety margins were estimated based on the projected mAb1 human pharmacokinetics and the observed toxicological and toxicokinetic data from the mAb1 Good Laboratory Practice (GLP)-compliant 13-week cynomolgus monkey study, with the highest tested dose of 100 mg/kg IV every week (Q1W) being the no-observed-adverse-effect-level (NOAEL).

By comparing exposures (i.e., AUC_{inf}), the safety margin for the 200 mg starting dose was estimated to be >19-fold and for the 1800 mg top dose was estimated to be >2.1-fold. These safety margin estimates, including both Cₘₐₓ and AUC_{0-inf}, supported the testing of these doses in the study for mAb1 (Table 3A).

In this study, members of Group 2a received SC injections, to evaluate the absolute subcutaneous bioavailability of mAb1. Characterization of the pharmacokinetic profile with SC administration of mAb1 informed possible later clinical development phases.

Dose escalation for Group 2 through Group 5 took place for the subsequent dose group after evaluating at least 2 weeks (through Week 2 (Day 15)) of safety data of all subjects from the preceding dose group. In addition, within each dose group, full enrollment proceeded after sentinel dosing. Planned dose levels were subject to change after review of pharmacokinetic, safety and tolerability data from all subjects from the preceding group(s) in conjunction with available data from beyond 2 weeks in previously dosed group(s).

TABLE 3A
Estimated dose-based and exposure-based multiples
for proposed clinical doses of mAb1 for Study C.

| | | | | AUC-Based^{c} | |
|---|---|---|---|---|---|
| | | Dose-Based^{a} | Cₘₐₓ-Based^{b} | AUC_{tau, ss} | AUC_{tau, ss} × 4 |
| Human Dose | | NOAEL = 100 | First Dose 2250 | 26,700 μg · | 106,800 μg · |
| (mg/Kg) | | mg/Kg | μg/mL | day/mL | day/mL |
| 50 mg | (0.83 mg/kg) | 120 | 80-124 | 76-84 | 308-336 |
| 200 mg | (3.3 mg/kg) | 30 | 20-31 | 19-21 | 77-84 |
| 400 mg | (6.7 mg/kg) | 15 | 9.8-15 | 9.6-10 | 38-41 |
| 800 mg | (13.3 mg/kg) | 7.5 | 4.9-7.6 | 4.8-5.2 | 19-21 |
| 1200 mg | (20 mg/kg) | 5.0 | 3.2-5.0 | 3.2-3.5 | 13-14 |
| 1800 mg | (30 mg/kg) | 3.3 | 2.2-3.4 | 2.1-2.3 | 8.5-9.3 |

^{a}Calculated as the ratio of human equivalent dose at the NOAEL (100 mg/kg) to proposed human dose.
^{b}Calculated as the ratio of the observed Cₘₐₓ after first administered dose at the NOAEL to the predicted human Cₘₐₓ.
^{c}Two AUC-based exposure multiples were calculated: (i) ratio of the AUCₜ at steady-state in the GLP toxicology study at the NOAEL (that is, monkey AUC₀₋₁₆₈ after the 13^{th} dose) to the predicted human exposure AUC_{inf} after a single dose and (ii) ratio of four times the AUCₜ at steady-state (that is, monkey AUC_{0-168 h} after the 13^{th} dose multiplied by 4) in the GLP toxicology study at the NOAEL to the predicted human exposure AUC_{inf} after a single dose. The factor of 4 adjustment was applied to account for differences in dosing frequency in the GLP toxicology study versus planned clinical dosing frequency.

This study was conducted in a double-blind manner with respect to mAb1 and matching placebo.

### Eligibility Criteria

Subjects who met all of the following criteria were included in the study. Anything other than a positive response to the questions below resulted in exclusion from study participation.

### Consent:

1. Subjects voluntarily signed and dated an informed consent, approved by an institutional review board (IRB), prior to the initiation of any screening or study-specific procedures.

### Demographic:

2. Subject was between 18 and 55 years of age inclusive at the time of screening.
3. Subject had a Body Mass Index (BMI) ≥18.0 to ≤29.9 kg/m² after rounding to the tenths decimal. BMI was calculated as weight in kg divided by the square of height measured in meters.

### Contraception Eligibility and Guidance:

4. If female, subject must have been either:
Permanently surgical sterile (bilateral oophorectomy, bilateral salpingectomy or hysterectomy) OR Postmenopausal, defined as age ≤55 years with no menses for 12 or more months without an alternative medical cause AND a follicle-stimulating hormone (FSH) level >40 IU/L.
5. Female subjects who were not breastfeeding, pregnant or planning a pregnancy.
6. Male subjects who were sexually active with a female partner of childbearing potential must agree to use condoms, from Week 0 (Day 1) through the Day 140 Follow-up phone call (Day 112 for Group 6) even if the male subject has undergone a successful vasectomy.
7. Male subjects who were not considering fathering a child or donating sperm during the study from Week 0 (Day 1) through the Day 140 (Day 112 for Group 6) Follow-up phone call.

### Laboratory Assessments and ECG Criteria

8. Laboratory values that met the following criteria:
total bilirubin, serum aspartate transaminase (AST) and alanine transaminase (ALT) ≤the upper limit of normal (ULN) at the screening visit and upon initial confinement.
negative test result for hepatitis A virus immunoglobulin M (HAV-IgM), hepatitis B surface antigen (HBsAg), hepatitis C virus (HCV) antibody (Ab) and human immunodeficiency virus antibody (HIV Ab) at screening visit.
absolute neutrophil count (ANC), absolute lymphocyte count (ALC), and platelets ≥the lower limit of normal (LLN), white blood cell count (WBC) ≥3.60 thousand/mm³ and hemoglobin ≥11.0 g/dL for females or 13.0 g/dL for males at the screening visit and upon initial confinement.
negative screen for drugs of abuse or alcohol or cotinine at screening and upon initial confinement.
for all female subjects who were not postmenopausal; a negative urine pregnancy test at the screening visit and a negative serum pregnancy test upon initial confinement and prior to the first dose of study drug.
for post-menopausal women, no pregnancy test was required.
no clinically significant abnormality in chest x-ray, including calcified granuloma and/or pleural scarring (chest x-ray was only required for any subject with one or more positive TB risk factors).
no other laboratory results that the investigator determined were clinically significant.
9. No clinically significant ECG abnormalities including:
no evidence of 2nd or 3rd degree AV block at screening visit and upon initial confinement.
QT interval corrected for heart rate (QTc) using Fridericia's correction formula (QTcF) is ≤430 msec (males) or ≤450 msec (females) at screening visit and upon initial confinement.

### Subject History/Physical Exam and Vital Signs

10. A condition of generally good health based on the results of a medical history, physical examination, vital signs, laboratory profile, and a 12-lead ECG.
11. No history of epilepsy, any clinically significant cardiac, respiratory (except mild asthma as a child), renal, hepatic, gastrointestinal, hematologic or psychiatric disease or disorder, or any uncontrolled medical illness.
12. No history of any clinically significant sensitivity or allergy to any medication or food, including no history of allergic reaction or anaphylaxis to therapeutic proteins, vaccines, or other parenteral treatments.
13. No evidence of dysplasia or history of malignancy (including lymphoma and leukemia) other than successfully treated non-metastatic cutaneous squamous cell, basal cell carcinoma or localized carcinoma in situ of the cervix.
14. No history or evidence of active tuberculosis (TB) disease or latent TB infection.
15. Negative TB skin test or QuantiFERON-TB Gold In-Tube (GIT assay) within 90 days prior to study drug administration.
16. No history of any clinically significant illness/infection/major febrile illness, hospitalization, or any surgical procedure within 30 days prior to the first dose of study drug.
17. Had not donated blood (including plasmapheresis), lost ≥550 mL blood volume, or received a transfusion of any blood product within 8 weeks prior to study drug administration.
18. No consumption of alcohol, grapefruit products, Seville oranges, starfruit products or quinine/tonic water within the 72-hour period prior to study drug administration.
19. No use of tobacco or nicotine-containing products within 180 days prior to the first dose of study drug.
20. No history of clinically significant (per investigator's judgment) drug or alcohol abuse within the last 6 months.
21. No history of an allergic reaction or significant sensitivity to constituents of the study drug (and its excipients) and/or other products in the same class.
22. Was not currently enrolled in another interventional clinical study.
23. Had not been previously enrolled in this study.
24. In the opinion of the investigator, this subject was a suitable candidate for enrollment in the study.

### Concomitant Medications

25. Subject must not have been treated with any investigational drug within 30 days or 5 half-lives of the drug (whichever is longer) prior to the first dose of study drug or is currently enrolled in another clinical study.
26. Subject must not have received any live vaccine within 4 weeks prior to the first dose of study drug, or expected need of live vaccination during study participation including at least 4 weeks after the last dose of study drug.
27. Subject must not require any over-the-counter and/or prescription medication, vitamins and/or herbal supplements on a regular basis.
28. Subject must not have used any medications, vitamins and/or herbal supplements within the 2-week period prior to study drug administration.
29. No receipt of any drug by injection within 30 days or within a period defined by 5 half-lives, whichever was longer, prior to study drug administration.
30. No exposure to antibody-based immunotherapy or previous enrollment in antibody-based immunotherapy clinical trials within 30 days or within a period defined by 5 half-lives, whichever was longer, prior to the first dose of study drug.

### Evaluation

The study results were evaluated by specific safety endpoints described herein. The safety endpoints were frequency and severity of adverse events (AEs), where safety evaluations included vital signs measurements, physical examinations, electrocardiogram (ECG) variables, and clinical laboratory testing throughout the study.

### Pharmacokinetic Endpoints

Serial blood samples were collected throughout the study for the evaluation of pharmacokinetic and immunogenicity endpoints.

The following pharmacokinetic parameter values for mAb1 were estimated using noncompartmental methods: maximum observed serum concentration (Cₘₐₓ) and time to Cₘₐₓ (Tₘₐₓ) following a single dose of mAb1, area under the serum concentration-time curve (AUC) from time 0 to the time of last measurable concentration (AUCₜ), AUC from time 0 to infinite time (AUC_{inf}), terminal phase elimination rate constant (β), and terminal phase elimination half-life (t_{1/2}). Blood samples were used to evaluate target engagement to inform pharmacokinetics and pharmacodynamics and to explore changes in biologic activities that may be associated with the mechanism of action. Samples (blood) were collected at specific time points.

Pharmacodynamic endpoints included the following were evaluated as changes from baseline (pre-dose) to post-dose (i.e., after study drug administration).
1. Changes in α4β7 receptor coverage by study drug on CD4+ T cells.
2. Changes in percentage of peripheral 07+ T cells.
3. Changes in lymphocyte counts (e.g., T cells, B cells, and NK [TBNK] cells including CD4 and CD8 counts).
   Clinical Pharmacokinetics of mAb1 in Healthy Subjects

The PK of mAb1 was evaluated in healthy adult subjects in the Phase 1 SAD Study C. The PK parameters from each group included: Group 1 (200 mg IV infusion), Group 2 (400 mg IV infusion), Group 3 (800 mg IV infusion), Group 4 (1200 mg IV infusion), Group 5 (1800 mg IV infusion), Group 6 (50 mg IV infusion), and Group 2a (800 mg SC injection). The PK parameters were summarized in Table 3B. Serum samples were assayed for mAb1 concentrations using validated immunoassay methods with LLOQ of 3.9 ng/mL.

Overall, the disposition of mAb1 in healthy subjects was similar to a typical monoclonal antibody. The terminal half-life ranged from approximately 18 to 25 days for doses ranging from 800 mg to 1800 mg IV. The linear PK was observed over a dose range from 400 mg to 1800 mg IV.

TABLE 3B
The Geometric Mean (Mean, % CV) Pharmacokinetic Parameters of
mAb1 after Single IV or SC Administration in Healthy Subjects.

| | G1 | G2 | G3 | G4 | G5 | G6 | G2a SC |
|---|---|---|---|---|---|---|---|
| | 200 mg IV | 400 mg IV | 800 mg IV | 1200 mg IV | 1800 mg IV | 50 mg IV | 800 mg |
| PK parameters | (N = 6) | (N = 6) | (N = 6) | (N = 6) | (N = 5^{a}) | (N = 6) | (N = 6) |
| Cₘₐₓ | 67.4 | 156 | 312 | 416 | 579 | 18.4 | 76.3 |
| (μg/mL) | (68.8, 22) | (158, 15) | (325, 34) | (420, 15) | (588, 21) | (18.6, 16) | (87.8, 48) |
| AUCₜ | 765 | 2120 | 5190 | 8490 | 9880 | 177 | 2630 |
| (μg · day/mL) | (785, 25) | (2260, 36) | (5310, 22) | (8630, 21) | (10400, 38) | (183, 30) | (3030, 50) |
| AUC_{inf} | 767 | 2150 | 5370 | 8860 | 10300 | 178 | 2760 |
| (μg · day/mL) | (787, 25) | (2310, 39) | (5510, 23) | (9010, 21) | (10800, 39) | (185, 30) | (3240, 53) |
| Tₘₐₓ | 5 | 6 | 4 | 5 | 3.5 | 2.5 | 120 |
| (h)^{b} | (2-14) | (2-6) | (2.5-4) | (2-14) | (3-5) | (2-4) | (48-168) |
| t_{1/2} | 9.47 | 12.2 | 21.5 | 24.8 | 18.3 | 5.69 | 16.0 |
| (day)^{c} | (6.41) | (5.50) | (5.26) | (4.72) | (6.13) | (3.93) | (17.5) |
| Cₘₐₓ/Dose | 0.337 | 0.390 | 0.389 | 0.347 | 0.322 | 0.368 | 0.0953 |
| (μg/mL/mg) | (0.344, 22) | (0.394, 15) | (0.407, 34) | (0.350, 15) | (0.327, 21) | (0.371, 16) | (0.110, 48) |
| AUCₜ/Dose | 3.82 | 5.29 | 6.49 | 7.08 | 5.49 | 3.53 | 3.28 |
| (μg · day/mL/mg) | (3.92, 25) | (5.64, 36) | (6.64, 22) | (7.19, 21) | (5.77, 38) | (3.66, 30) | (3.79,50) |
| AUC_{inf}/Dose | 3.83 | 5.37 | 6.71 | 7.38 | 5.70 | 3.56 | 3.45 |
| (μg · day/mL/mg) | (3.94, 25) | (5.77, 39) | (6.88, 23) | (7.51, 21) | (6.01, 39) | (3.70, 30) | (4.04,53) |

G = group;
AUC_{inf} = AUC from time 0 to infinity;
AUCₜ = AUC from time 0 until the last observable concentration;
Cₘₐₓ = maximum observed concentration;
IV = intravenous;
N = the number of subjects dosed with mAb1;
Tₘₐₓ = time to maximum observed concentration;
t_{1/2} = apparent elimination half-life
^{a}N = 5 for Cₘₐₓ, Tₘₐₓ and Cₘₐₓ/Dose; N = 4 for all other parameters in 1800 mg IV group.
One subject prematurely discontinued and had mAb1 concentration data only up to Day 28.
^{b}Median (minimum-maximum)
^{c}Harmonic mean (Pseudo SD)

### ADA Assessment in Healthy Subjects

The treatment-emergent anti-drug antibody (ADA) incidence after a single dose of mAb1 observed in Study C is summarized in Table 3C. The LLOQ of the immunogenicity assay was 10 titer units. The following criteria defined an ADA positive subject within the study period:

For subjects who had an unquantifiable ADA titer value (i.e., <LLOQ) or missing assessment at baseline (i.e., prior to mAb1 dose), subject were classified as an ADA positive subject if a quantifiable ADA titer value was observed at one or more time points post baseline.

For subjects who had a quantifiable ADA titer value (i.e., ≥LLOQ) at baseline (i.e., prior to mAb1 dose), subject was classified as an ADA positive if 4-fold or greater increase in ADA titer values was observed at post-baseline visits relative to the baseline. In Study C, no subject had a quantifiable ADA titer value at baseline.

TABLE 3C
Summary of ADA Incidence After Single Dose of mAb1 in Study C in Healthy Subjects.

| Dose Group | 50 mg IV | 200 mg IV | 400 mg IV | 800 mg IV | 800 mg SC | 1200 mg IV | 1800 mg IV |
|---|---|---|---|---|---|---|---|
| (N) | (N = 6) | (N = 6) | (N = 6) | (N = 6) | (N = 6) | (N = 6) | (N = 3)^{a} |
| ADA Positive | 4/6 | 4/6 | 2/6 | 1/6 | 1/6 | 2/6 | 1/3 |
| (n/N) | (66.7%) | (66.7%) | (33.3%) | (16.7%) | (16.7%) | (33.3%) | (33.3%) |

ADA = Anti-drug antibody;
IV = intravenous;
N = subjects receiving active drug and had completed at least 5 ADA assessment visits within the study;
n = ADA positive subjects;
SC = subcutaneous
^{a}a total of five subjects received active drug in 1800 mg IV cohorts. Two subjects had insufficient data to conclude their immunogenicity status within the study period.

The impact of ADA on mAb1 exposures was assessed and showed that ADA positive subjects tended to have lower exposures compared to ADA negative subjects for subjects in the <800 mg dose groups. In the 1200 mg IV dose group, one subject had a transient ADA titer value of 10 titer (equal to the LLOQ) detected at only one ADA assessment visit. In the 1800 mg IV dose group, one subject had a transient ADA titer value of 10 titer detected at only one ADA assessment visit. The immunogenicity analysis suggests that the types of ADA may impact on mAb1 exposures.

Overall PK Summary for mAb1 in Healthy Subjects and PLWH (Studies C and D)

Summarized below were key PK findings based on analysis of data from the mAb1 Phase 1a Study C in healthy subjects and Phase 1b Study D in PLWH:

The disposition of mAb1 in healthy subjects was similar to a typical monoclonal antibody. The terminal half-life ranged from approximately 18 to 25 days for doses ranging from 800 mg to 1800 mg IV. The linear PK was observed over a dose range from 400 mg to 1800 mg IV.

ADA analysis in healthy subjects showed a trend in dose <800 mg cohorts ADA positive subjects had lower exposures compared to ADA negative subjects. The types of ADA may impact on mAb1 exposures.

The accumulation ratios for Cₘₐₓ and AUCₜₐᵤ for HIV aviremic adult subjects who received two doses of 800 mg IV Q4W were 1.07 and 1.516, respectively. The accumulation ratios for Cₘₐₓ and AUCₜₐᵤ for HIV aviremic adult subjects who received three doses of 1600 mg IV Q4W were 1.723 and 1.967, respectively.

When PK parameters for healthy subjects, viremic, and aviremic PLWH following a single dose of 800 mg IV mAb1 were compared, viremic PLWH showed the lowest AUC_{inf} and shortest terminal half-life among the three populations.

Pharmacodynamics of mAb1 in Healthy Subjects and PLWH (Studies C and D)

Baseline α4β7 expression in Study C and Study D:

α4β7 expression was measured with the anti-α4β7 antibody. Percentage of α4β7+ cells were comparable across CD4+ and CD8+ T cell subsets (total, naïve or central/transient memory) across treatment arms in healthy subjects and PLWH at baseline (Table 3D). α4β7 expression, as measured by Molecules of Equivalent Soluble Fluorochrome (MESF) flow cytometry, was higher on the CD4+ T cell central/transient memory population (CD28+/CD95+). The mAb1 achieved immediate saturation of α4β7 integrin receptors on CD4 memory cells after completion of each single dose infusion and within 2 hours of SC dosing. Nearly complete α4β7 integrin receptor saturation (>90%) was achieved at all dose levels, and the duration of α4β7 receptor saturation was concentration-dependent, with full receptor saturation sustained for at least 28 days post dosing in all dose groups. No α4β7 receptor saturation was observed for subjects receiving placebo.

The mAb1-mediated receptor internalization was measured with a non-competing antibody targeting the β7 subunit. A reduction in 37 expression was observed on CD4 and CD8 cells, which indicated mAb1-mediated receptor internalization. The mAb1-mediated 37 internalization was observed at all doses and appeared partial.

TABLE 3D
Baseline α4β7 Integrin Expression in Studies
C and D as Measured by MESF Flow Cytometry.

| | Treatment | PBO IV | 50 mg | 200 mg | 400 mg | 800 mg | 1200 mg | 1800 mg |
|---|---|---|---|---|---|---|---|---|
| | Group | or SC | IV | IV | IV | IV | IV | IV |
| Study C | N^{a} | 13 | 5 | 6 | 6 | 5 | 6 | 5 |
| | Median | 16.47 | 14.44 | 19.21 | 13.28 | 13.72 | 17.61 | 16.63 |
| | (SD) % | (5.65) | (2.82) | (2.14) | (4.34) | (3.24) | (2.63) | (3.17) |
| | α4β7 on | | | | | | | |
| | CD4+ Memory | | | | | | | |
| | cells^{b} | | | | | | | |
| | Median (SD) | 13,846 | 12,753 | 14,166 | 12,806 | 11,575 | 12,824 | 12,815 |
| | α4β7 MESF on | (1960) | (3510) | (2523) | (1131) | (853) | (828) | (680) |
| | CD4+ | | | | | | | |
| | Memory cells^{b} | | | | | | | |

| | Treatment | Viremic | Viremic | Aviremic | Aviremic | Aviremic |
|---|---|---|---|---|---|---|
| | Group | 800 mg | 1600 mg | PBO | 800 mg | 1600 mg |
| Study D | N^{a} | 5 | 3 | 10 | 12 | 10 |
| | Median | 16.64 | 16.66 | 12.41 | 13.32 | 14.75 |
| | (SD) % α4β7 | (5.60) | (0.55) | (3.86) | (3.35) | (4.95) |
| | on | | | | | |
| | CD4+ Memory | | | | | |
| | cells^{b} | | | | | |
| | Median (SD) | 11,939 | 10,769 | 12,151 | 12,866 | 12,325 |
| | α4β7 MESF on | (1127) | (940) | (1794) | (2434) | (2807) |
| | CD4+ | | | | | |
| | Memory cells^{b} | | | | | |

^{a}N = number of subjects with available baseline data
^{b}CD4+ T cell central/transient memory population (CD28+/CD95+) 1

### Exposure-Response Relationship in Healthy Subjects (Study C)

The PD results indicate that mAb1 achieved immediate saturation of α4β7 integrin receptors expressed on CD4+ and CD8+ T cells in peripheral blood in healthy subjects after completion of each single dose infusion. PK/PD relationship using peripheral CD4+ T cell central/transient memory population as representative for receptor saturation showed nearly complete α4β7 integrin receptors saturation (>90%) on peripheral CD4+ T cell central/transient memory population was achieved at all dose levels, and the duration of α4β7 integrin receptors saturation was mAb1 concentration dependent, with full receptor saturation sustained for at least 28 days across all IV doses. Recovery of free α4β7 integrin receptors on CD4+ T cell central/transient memory population was associated with decreasing mAb1 concentrations. The PK/PD relationship was consistently seen across all dose levels. Nearly complete α4β7 integrin receptors saturation (>90%) was achieved at EC₉₀ of approximately 3 μg/mL mAb1 concentrations.

### Example 4 Study D

A phase 1b study was conducted to evaluate the safety, pharmacokinetics, and pharmacodynamics of single and multiple doses of an anti-α4β7 integrin monoclonal antibody (mAb1) in People Living With HIV-1 (PLWH).

### Study Objectives:

The main objectives of this study included evaluating the safety and tolerability of the full-length anti-α4β7 integrin monoclonal antibody mAb1 having heavy chains shown as SEQ ID NO:18 and may also have a C-terminal lysine truncated heavy chain of SEQ ID NO: 21 and light chains shown as SEQ ID NO:19 single ascending doses in HIV viremic subjects, evaluating the safety and tolerability of multiple ascending doses in subjects who were HIV virally suppressed (aviremic) on antiretroviral therapy (ART), and evaluating the pharmacokinetics and immunogenicity after single ascending doses or multiple ascending doses of mAb1 in all subjects.

### Background and Rationale

In this study, anti-α4β7 integrin antibody mAb1 was evaluated in persons living with HIV-1 (PLWH).

The safety, tolerability, pharmacokinetic, and pharmacodynamic profiles of mAb1 were determined in HIV viremic subjects and HIV aviremic subjects allowed for the selection of dose(s) to be further evaluated in a proposed Phase 2 study.

### Investigational Plan

This was a Phase 1b study with two parts in adult PLWH, including an open-label, single-ascending, intravenous (IV) dose study of mAb1 in viremic subjects with no previous ART experience or who have been off ART for >3 months prior to screening (Part A), and a randomized, double-blind, placebo controlled (for IV dosing) or open-label (for subcutaneous [SC] dosing) multiple-ascending dose study of mAb1 in aviremic subjects who were on stable ART (Part B). Part A and Part B of the study were conducted in parallel. Approximately 52 subjects were enrolled in this study.

In Part A, two dose levels of mAb1 were administered by IV infusion and were assessed in up to 12 viremic subjects with no previous ART experience or who have been off of ART for >3 months prior to screening. Sequential enrollment was performed to enroll up to 6 subjects each for the 800 and 1600 mg mAb1 IV dose groups with no placebo control. In Part A, subjects remained off ART through Day 28, unless any of the following occurred:
two dose levels of HIV-related/AIDS-associated opportunistic infection (OI) requiring rapid initiation of ART (e.g., pneumocystis pneumonia (PCP), meningitis);
pregnancy; and
confirmed decline in CD4 count >30% from baseline or confirmed absolute CD4+ T-cell count <250 cells/mm³.

Subject might initiate ART (prescribed by the treating physician; ART were not provided in this study) after Day 28 at the discretion of the subject and their physician.

In Part B, multiple-ascending doses of mAb1 were evaluated in subjects who were aviremic in the presence of ART. Part B included cohorts of subjects receiving study drug via intravenous (IV) infusion or subcutaneous (SC) injection.

For the IV infusion cohort, two dose levels of mAb1 or placebo were administered on Days 1, 29, and 57 (every 4 weeks [Q4W]) and were assessed in approximately 30 subjects. Sequential enrollment was performed to enroll approximately 15 subjects each for the 800 and 1600 mg mAb1 IV dose groups. Within each IV dose group, subjects were randomized in a 2:1 ratio to active or placebo. For the IV infusion cohort, the study site and subjects remained blinded for the duration of the study.

For the SC injection cohort, 800 mg of mAb1 was administered on Days 1, 29, and 57 (Q4W) and assessed in approximately 10 subjects. The SC injection cohort did not have a placebo control. Subjects in Part B remained on ART throughout the study.

### Key Eligibility Criteria:

Subjects met all of the following criteria in order to be included in the study. Anything other than a positive response to the questions below resulted in exclusion from study participation. A subject who failed screening may be re-screened once at investigator's discretion. If the subject failed a screening a second time, they were excluded from the study.

### Consent:

1. Subjects voluntarily signed and dated an informed consent, approved by an independent ethics committee (IEC)/institutional review board (IRB), prior to the initiation of any screening or study-specific procedures.

### Demographic:

2. Subject was between 18 and 65 years of age inclusive at the time of screening.
3. Subject had a Body Mass Index (BMI) is ≥18.0 to ≤35.0 kg/m² after rounding to the tenths decimal. BMI was calculated as weight in kg divided by the square of height measured in meters.
4. Subject weighed at least 35 kg.
5. Subjects were in a condition of general good health based upon the results of a medical history, physical examination, vital signs, laboratory profile and a 12-lead ECG.
6. Subject did not have clinically significant laboratory values at screening that would pose undue risk for the subject or interfere with safety assessments (per the investigator).

### Contraception Eligibility and Guidance:

7. Subjects had a negative serum pregnancy test at screening and negative urine pregnancy test at the Pre-Dose Visit (Part A and Part B) for females of childbearing potential only. Note, females of non-childbearing potential did not require any pregnancy tests. Non-childbearing potential is defined as:
a) Postmenopausal, age >55 years with no menses for 12 or more months without an alternative medical cause.
b) Postmenopausal, age ≤55 years with no menses for 12 or more months without an alternative medical cause and a follicle-stimulating hormone (FSH) level >40 IU/L.
c) Permanently surgically sterile (bilateral oophorectomy, bilateral salpingectomy, or hysterectomy).
8. Female subjects of childbearing potential agreed to use an effective barrier method of protection during sexual activity from Week 0 (Day 1) through the last study visit for the purposes of prevention of HIV transmission. In addition, female subjects of childbearing potential used an additional protocol-specified effective method of contraception for the purposes of birth control from Week 0 (Day 1) through the last study visit (including for at least 24 weeks after the last dose of study drug for subjects who discontinue the study early.
9. Female subjects of non-childbearing potential agreed to use an effective barrier method of protection during sexual activity from Week 0 (Day 1) through the last study visit to prevent HIV transmission. Non-childbearing potential is defined in eligibility criterion.
10. Female subjects of childbearing potential gave consent to abide by contraception requirements.
11. Female subjects were not pregnant, breastfeeding, or considering becoming pregnant during the study.
12. Male subjects who were sexually active with a female partner of childbearing potential agreed to use condoms from Week 0 (Day 1) through the last study visit for the purposes of both contraception and prevention of HIV transmission, even if the male subject had undergone a successful vasectomy.
13. Male subjects were prohibited from considering fathering a child or donating sperm during the study from Week 0 (Day 1) through the last study visit.
   Laboratory assessment
14. Laboratory Assessments and ECG Criteria:

Laboratory values at screening met the following criteria:
a) Negative HIV-1 Ab test;
b) Negative test result for hepatitis B surface antigen (HBsAg);
c) Absence of current hepatitis B virus (HBV) infection on screening tests, defined as: a positive hepatitis B surface antigen (HBsAg), or HBV DNA >lower limit of quantitation (LLOQ) in subjects with isolated positive anti-hepatitis B core (HBc) antigen (i.e., negative HbsAg and anti-hepatitis B surface Ab [HBs]);
d) Negative test result for hepatitis C virus (HCV) antibody (Ab) (may include subjects with a positive HCV Ab who have documented completion of a recommended HCV treatment regimen at least 12 weeks prior to screening and a documented negative HCV RNA at screening)
e) Absence of current HCV infection on screening tests defined as: detectable HCV RNA result if HCV Ab positive. Subjects with a history of hepatitis C who achieved sustained virologic response (SVR), or no detectable amount of HCV, after 12 weeks of treatment (SVR12) after anti-viral therapy may be enrolled if all eligibility criteria are met.
f) CD4+ count of ≥350 cells/μL at screening and at least once during the 48 weeks prior to screening
g) Absolute neutrophil count (ANC) and absolute lymphocyte count (ALC) ≥the lower limit of normal (LLN)
h) Hemoglobin level ≥10 g/dL (males) and >9.5 g/dL (females) (National Institutes of Health (NIH) Division of AIDS (DAIDS) Table for Grading the Severity of Adult and Pediatric Adverse Events ([DAIDS][v2.1][July 2017])
i) Calculated creatinine clearance (using Cockcroft-Gault method) of ≥60 mL/min
j) Platelets ≥125,000 cells per mm
k) ALT and aspartate aminotransferase (AST) ≤1.25×upper limit of normal (ULN)
l) Total bilirubin ≤1.1×ULN (unless taking atazanavir or known history of Gilbert's Syndrome)
m) International normalized ratio (INR) ≤1.1×ULN
15. Negative screen for drugs of abuse and alcohol at screening. Subjects with a positive marijuana screen may be included after evaluation by the investigator that the use would not interfere with adherence to study requirements, and that usage is not on a regular or chronic basis.
16. No other laboratory results that the investigator determines are clinically significant.
17. Absence of clinically significant abnormality detected on electrocardiogram (ECG) regarding rate, rhythm, or conduction (e.g., QT interval corrected for heart rate using Fridericia's formula [QTcF] should be <450 msec for males and <460 msec for females).

### Subject History

### Disease (HIV) Activity-Part a (Viremic) Subjects

18. The following HIV-specific laboratory parameters must be met at screening:
A) Positive test result for anti-HIV Ab at screening.
B) Plasma HIV-1 RNA between 1,000-200,000 copies/mL at screening.
19. Were naive to ART or have been off of ART for: 12 weeks or 5 half-lives of the drug (whichever is longer) prior to screening with documentation of at least one plasma HIV-1 RNA measurement greater than or equal to the lower limit of quantification (LLOQ) during the off-ART period.
20. Willing to hold off on initiation of ART throughout the screening period and until 4 weeks after dosing.

### Disease (HIV) Activity—Part B (Aviremic) Subjects

21. The following HIV-specific laboratory parameters were met at screening:
A) Positive test result for anti-HIV Ab at screening;
B) Subject had plasma HIV-1 RNA below the lower limit of quantification at screening; and at least 24 weeks prior to screening. A single unconfirmed “blip” (i.e., plasma HIV-1 RNA value greater than or equal to the lower limit of quantification but <200 copies/mL) was allowed if preceded and followed by values below the lower limit of quantification (by an approved plasma HIV-1 RNA quantitative assay including but not limited to COBAS® Ampliprep/COBAS® Taqman HIV-1 Test v2.0).
22. Must have been HIV-1-infected on ART for at least 48 weeks prior to screening and on current ART regimen for at least 12 weeks prior to screening.

### Non-HIV History—Part A and Part B

23. No history or ongoing diagnosis of AIDS-defining illness.
24. No history of or active immunodeficiency (other than HIV).
25. No active autoimmune disease or history of autoimmune disease that required systemic treatment (i.e., with use of disease-modifying agents, corticosteroids, or immunosuppressive drugs). Replacement hormonal therapy (e.g., T4) was not considered a form of systemic treatment.
26. Subject did not have had prior therapy/exposure to mAb1.
27. Subject was not exposed to Ab-based immunotherapy or previous enrollment in antibody-based immunotherapy clinical trials within 4 weeks or within a period defined by 5 half-lives, whichever is longer, prior to the first dose of study drug.
28. Subject had no clinically significant medical disorders (other than HIV-1 infection) that might expose the subject to undue risk of harm, confound study outcomes or prevent the subject from completing the study, including but not limited to:
A) Significant or unstable cardiac disease (e.g., congestive heart failure, angina, myocardial infarction;
B) Significant pulmonary disease (e.g., COPD, asthma requiring systemic therapy);
C) Significant neurologic disease (e.g., stroke, dementia);
D) Chronic active infectious disease (except for HIV) (e.g., sinusitis, chronic chest infection);
E) Chronic liver disease; and
F) Poorly controlled diabetes mellitus.
29. Subject had no history of any clinically significant illness/infection/major febrile illness, hospitalization, or any surgical procedure within 4 weeks prior to the first dose of study drug.
30. Subject had no active or suspected malignancy or history of malignancy (other than basal cell skin cancer or cervical carcinoma in situ) in the past 5 years.
31. Subject had no history or evidence of active tuberculosis (TB) disease or untreated latent TB infection at screening.
32. Subject had no history of positive TB skin test or interferon gamma release assay (IGRA) which was considered clinically significant by the investigator.
A) Subject with a history of a positive TB skin test or IGRA must have documentation of completion of a Centers for Disease Control and Prevention (CDC) recommended treatment course for latent TB. Any subject with suspicion for or diagnosis of active TB was excluded;
B) Subject must have a negative result for an IGRA at screening unless subject had a documented negative IGRA result within 12 weeks prior to screening and nothing has changed in subject's medical history to warrant a repeat test at screening; and
C) If positive IGRA at screening, subject must have no clinically significant abnormality in chest x-ray, such as calcified granuloma and/or pleural scarring.
33. Subject had no history of a severe, life-threatening, or significant sensitivity to any excipients of the study drug.
34. Subject had no history of any clinically significant sensitivity or allergy to any medication or food, including no history of allergic reaction or anaphylaxis to therapeutic proteins, vaccines, or other parenteral treatments.
35. Subject had not donated blood (including plasmapheresis), lost ≥550 mL blood volume, or received a transfusion of any blood product within 8 weeks prior to study drug administration.
36. Subject had no known psychiatric or substance abuse disorders that would interfere with adherence to study requirements.
37. Subject was not currently enrolled in another interventional clinical study.
38. Has not been previously enrolled in this study.
39. In the opinion of the investigator, this subject was a suitable candidate for enrollment in this study.

### Concomitant Medications

40. Subjects must not have received immunomodulatory (e.g., interleukins, interferons, tumor necrosis factor modifiers) or immunosuppressive (including IV/PO steroids at any dose, but excluding steroids that were inhaled or topical) therapy within 24 weeks prior to the first dose of study drug.
41. Subject had not been treated with any investigational drug within 24 weeks prior to the first dose of study drug.
42. Subject had not received any live vaccine within 4 weeks prior to the first dose of study drug or expected need of live vaccination during study participation including at least 4 weeks after the last dose of study drug.

### COVID-19

43. Subject must not have shown signs/symptoms associated with COVID-19 infection.
44. Subject must have shown absence of current COVID-19 infection by any viral test (e.g., PCR, antigen) completed within 7 days prior to the Week 0 (Day 1) dose.

### Evaluation

The study results were evaluated by specific endpoints described herein.

Endpoints included the proportion of subjects who experienced study drug-related Grade 3 or higher adverse events and pharmacokinetics. Safety evaluations included adverse event monitoring including adverse events of special interest (AESIs): sustained decreases in CD4+ T cell counts, infusion-related reactions, HIV-related/acquired immune deficiency syndrome [AIDS]-associated opportunistic infections (OIs), hepatotoxicity, physical examinations, vital sign measurements, and clinical laboratory testing (including hematology and metabolic panels, plasma HIV-1 RNA, and CD4+ T cell counts) throughout the study.

Pharmacokinetic endpoints for mAb1 were as follows: Part A (single-ascending dose; SAD):
Pharmacokinetic parameters of mAb1 were estimated using non-compartmental methods and summarized.
Maximum observed serum concentration (Cₘₐₓ), the time to Cₘₐₓ (Tₘₐₓ), the area under the serum concentration-time curve (AUC) from time 0 to the time of last measurable concentration (AUCₜ), AUC from time 0 to infinite time (AUC_{inf}), terminal phase elimination rate constant (β), terminal phase elimination half-life (t_{1/2}).
Part B (multiple-ascending dose; MAD): pharmacokinetic parameters of mAb1 were estimated using noncompartmental methods and summarized.
Following the first, second, and third doses: Cₘₐₓ, Tₘₐₓ, observed concentration at the end of the 4-week dosing interval (C_{trough}), AUC during the 4-week dosing interval (AUCₜₐᵤ), and t_{1/2} (only for the third dose).
Immunogenicity of mAb1 was assessed by detecting anti-drug antibody (ADA). Neutralizing anti-drug antibody (nAb) were evaluated if useful for interpretation of the data.
Pharmacodynamic exploratory endpoint assessed α4β7 receptor coverage (%) by mAb1 evaluated as changes from baseline (prior to first study drug administration) and post baseline.

### Results:

The results of this trial are shown in Table 4A. There were no SAEs or deaths, and there was no discontinuation due to drug-related AE, ≥Grade 3 drug-related AE, or >Grade 2 AESIs (hepatoxicity, CD4 related decline, infusion or injection site reactions, HIV-related/AIDS-associated opportunistic infection). There were no new safety signals in this study, including no signal of hepatic injury.

In viremic people that received a single dose up to 1600 mg IV, the reported adverse events were mild or moderate and there was no grade 3 or higher adverse events reported.

In aviremic people that received up to three doses of 800 mg or 1600 mg IV, the majority of adverse events reported were mild. There were 2 grade 3 or higher adverse events reported (one worsening of diabetes and one tendon rupture) there were assessed as not related to the study drug.

In aviremic people that received up to three doses of 800 mg SC, also the majority of adverse events reported were mild. One adverse event of substance abuse that led to discontinuation of the study drug and of the study was reported and considered not related to the study drug. There was also a hepatic related adverse event nonrelated to the study drug reported in the same individual.

There were no clinically significant trends observed for baseline, post dose and change from baseline of hematology chemistry, urinalysis, and immunology (including CD4+ count). Potentially clinically significant Grade 2 laboratory abnormalities observed were considered not clinically significant as an AE by the PI and were not associated with any reported adverse events, except for one case of high glucose that was reported as adverse event worsening of diabetes. One grade 3 laboratory abnormality of liver enzyme (AST) elevation reported was associated to an adverse event of substance abuse and was considered as not related to the study drug.

There was no ART virological failure reported in the study.

### Vital Signs Summary

There was no clinically significant trend observed during the infusion monitoring period or in the study overall for any vital sign parameter in individuals who received study drug. There were no vital signs-related AEs reported.

### ECG Summary

ECGs were evaluated for entry into the study at baseline and could be performed as needed for any post-baseline safety evaluation. Only one subject had a post-baseline ECG, as part of an evaluation for an adverse event of chest discomfort (Grade 1, not related to study drug). The ECG was considered not clinically significant by the investigator. There were no ECG-related AEs reported.

TABLE 4A
Study D: Overview of Treatment Emergent Adverse Events Following Infusion with mAb1.

| | Part A: Viremic, | | | | | |
|---|---|---|---|---|---|---|
| | Single Dose | | Part B: Aviremic, Multiple Doses | | | |
| | 800 mg IV | 1600 mg IV | 800 mg IV | 1600 mg | 800 mg SC | Placebo IV |
| n (%) | (n = 5) | (n = 4) | (n = 12) | IV (n = 11) | (n = 11) | (n = 11) |
| Any AE | 4 | 2 | 8 | 10 | 3 | 10 |
| | (80%) | (50%) | (66.7%) | (90.9%) | (27.3%) | (90.9%) |
| Drug-related AE | 1 | 0 | 3 | 3 | 2 | 3 |
| | (20%) | | (25%) | (27.3%) | (18.2%) | (27.3%) |
| AE leading to | 0 | 0 | 0 | 0 | 1 | 0 |
| discontinuation of SD | | | | | (9.1%) | |
| Grade 3 or Higher | 0 | 0 | 1 | 1 | 1 | 0 |
| | | | (8.3%) | (9.1%) | (9.1%) | |
| Hepatic-related AESI | 0 | 0 | 0 | 0 | 1 | 0 |
| | | | | | (9.1%) | |

### Clinical Pharmacokinetics in PLWH

According to protocol, the PK of mAb1 in viremic subjects was investigated in the SAD cohort and PK of mAb1 in aviremic subjects was investigated in MAD cohorts in Study D. However, due to an FDA clinical hold in July 2021, no subjects in Group 3 (aviremic subjects) received three doses of 800 mg IV Q4W. Instead, seven subjects received one dose of 800 mg IV and five subjects received two doses of 800 mg IV Q4W of mAb1. The PK parameters by IV dose groups were summarized in Table 4B.

TABLE 4B
The geometric Mean (Mean, % CV) Pharmacokinetic Parameters of
mAb1 Following a Single Dose of 800 mg or 1600 mg IV in PLWH.

| Study Group | | |
|---|---|---|
| | | Study D Part B |
| | | Group 3 - subgroup |
| Study D Part A | Study D Part A | of subjects |
| Group 1 | Group 2 | received only 1 dose |
| 800 mg IV | 1600 mg IV | 800 mg IV |
| (N = 5) | (N = 4) | (N = 7) |
| Populations | | |
| Viremic PLWH | Viremic PLWH | Aviremic PLWH |

PK parameters

| | | | | | | |
|---|---|---|---|---|---|---|
| Cₘₐₓ | 287 | (289, 14) | 674 | (730, 41) | 275 | (277, 13) |
| (μg/mL) | | | | | | |
| AUCₜ | 2870 | (2950, 25) | 6440 | (6780, 37) | 3870 | (3960, 21) |
| (μg · day/mL) | | | | | | |
| AUC_{inf} | 2870 | (2950, 25) | 6440 | (6780, 37) | 3880 | (3970, 21) |
| (μg · day/mL) | | | | | | |
| Tₘₐₓ^{a} | 3.1 | (1.2-168.9) | 5.5 | (1.6-26.9) | 1.9 | (1.5-5.4) |
| (h) | | | | | | |
| t_{1/2}^{b} | 7.97 | (0.710) | 8.48 | (3.00) | 10.3 | (4.78) |
| (days) | | | | | | |
| Cₘₐₓ/Dose | 0.359 | (0.362, 14) | 0.421 | (0.456, 41) | 0.344 | (0.346, 13) |
| (μg/mL)/mg | | | | | | |
| AUCₜ/Dose | 3.59 | (3.68, 25) | 4.02 | (4.24, 37) | 4.84 | (4.95, 21) |
| (μg · day/mL)/mg | | | | | | |
| AUC_{inf}/Dose | 3.59 | (3.68, 25) | 4.02 | (4.24, 37) | 4.85 | (4.96, 21) |
| (μg · day/mL)/mg | | | | | | |

AUC_{inf} = AUC from time 0 to infinity;
AUCₜ = AUC from time 0 until the last observable concentration;
Cₘₐₓ = maximum observed concentration;
IV = intravenous;
N = the number of subjects dosed with mAb1;
PLWH = people living with HIV;
Tₘₐₓ = time to maximum observed concentration;
t_{1/2} = apparent elimination half-life
^{a}Median (minimum-maximum)
^{b}Harmonic mean (Pseudo SD)

The harmonic means t_{1/2} of mAb1 were approximately 8 and 8.5 days for HIV viremic PLWH who received a single dose of 800 mg IV or 1600 mg IV, respectively. The t_{1/2} was approximately 10 days for aviremic PLWH who received a single dose of 800 mg IV.

TABLE 4C
The Geometric Mean (Mean, % CV) Pharmacokinetic Parameters of mAb1 Following Two
Doses of 800 mg IV Q4W or Three Doses of 1600 mg IV Q4W in Aviremic Subjects.

| | Study Groups | | | | | | | |
|---|---|---|---|---|---|---|---|---|
| | Study D Part B Group 3, Aviremic | | | | Study D Part B Group 4, Aviremic | | | |
| | Subjects 800 mg IV Q4W, 2 Doses | | | | Subjects 1600 mg IV Q4W, 3 Doses | | | |
| | (N = 5) | | | | (N = 11) | | | |
| | 800 mg IV | | 800 mg IV | | 1600 mg IV | | 1600 mg IV | |
| | 1^{st} dose | | 2^{nd} dose | | 1^{st} dose | | 3^{rd} dose | |
| | (Week 0 | | (Week 4 | | (Week 0 | | (Week 8 | |
| | (Day 1)) | | (Day 29)) | | (Day 1)) | | (Day 57)) | |
| | (N = 5) | | (N = 5) | | (N = 11) | | (N = 11) | |
| Cₘₐₓ | 278 | (289, 32) | 295 | (301, 22) | 398 | (414, 30) | 685 | (692, 15) |
| (μg/mL) | | | | | | | | |
| AUCₜₐᵤ | 2860 | (2970, 31) | 4340 | (4450, 24) | 4750 | (4840, 21) | 9350 | (9520, 20) |
| (μg · day/mL) | | | | | | | | |
| Tₘₐₓ^{a} | 2.8 | (1.8-3.6) | 1.7 | (1.3-3.1) | 5.3 | (2.0-6.3) | 4.4 | (2.1-4.6) |
| (h) | | | | | | | | |
| t_{1/2}^{b} | — | | 15.2 | (4.96)^{c} | — | | 20.4 | (7.49)^{d} |
| (days) | | | | | | | | |
| Cₘₐₓ/Dose | 0.347 | (0.362, 32) | 0.369 | (0.377, 22) | 0.249 | (0.259, 30) | 0.428 | (0.432, 15) |
| (μg/mL)/mg | | | | | | | | |
| AUCₜₐᵤ/Dose | 3.58 | (3.71, 31) | 5.43 | (5.56, 24) | 2.97 | (3.02, 21) | 5.84 | (5.95, 20) |
| (μg · day/mL)/mg | | | | | | | | |

AUCₜₐᵤ = the area under plasma concentration-time curve over one dosing interval (28-days);
Cₘₐₓ = maximum observed concentration;
IV = intravenous;
N = the number of subjects dosed with mAb1;
Tmax = time to maximum observed concentration;
t_{1/2} = apparent elimination half-life
^{a}Median (minimum-maximum)
^{b}Harmonic mean (Pseudo SD)
^{c}Terminal half-life after the 2^{nd} 800 mg IV dose on Week 4 (Day 29)
^{d}Terminal half-life after the 3^{rd} 1600 mg IV dose on Week 8 (Day 57)

The PK parameters for aviremic PLWH who received two doses of mAb1 800 mg IV, or three doses of mAb1 1600 mg IV, were summarized in Table 4C. The harmonic mean t_{1/2} of mAb1 was 15.2 and 20.4 days for aviremic subjects who received two doses of 800 mg IV or three doses of 1600 mg IV. The summary pharmacokinetic parameters of mAb1 following three doses of 800 mg SC administration are shown in Table 4D. The harmonic mean t_{1/2} of mAb1 was 15 days for aviremic subjects who received three doses of 800 mg SC. The mAb1 showed some accumulation following Q4W dosing, and where at the highest dose level (1600 mg IV Q4W×3 doses), the AUCₜₐᵤ showed approximately a 2-fold increase.

TABLE 4D
Geometric Mean (Mean, % CV) Pharmacokinetic Parameters of mAb1
Following Three Doses Q4W of 800 mg SC in Aviremic Subjects

| | Study Group | | | |
|---|---|---|---|---|
| | Part B Group 3a, Aviremic Subjects 800 mg SC Q4W, 3 Doses | | | |
| | 800 mg SC | | 800 mg SC | |
| | 1^{st} dose | | 3^{rd} dose | |
| | (Week 0 (Day 1)) | | (Week 8 (Day 57)) | |
| | (N = 10) | | (N = 10) | |
| Cₘₐₓ (μg/mL) | 97.1 | (121, 102) | 107 | (110, 31) |
| Tₘₐₓ^{a} (h) | 167.9 | (4.3-336.5) | 168.1 | (120.0-193.0) |
| C_{trough} (μg/mL) | 43.0 | (43.2, 11)^{b} | 55.4 | (57.6, 31) |
| AUCₜ (μg · day/mL) | 1680 | (1690, 11) | 2150 | (2200, 25) |
| AUCₜₐᵤ (μg · day/mL) | 1660 | (1670, 8)^{b} | 2180 | (2250, 27) |
| t_{1/2}^{c} (days) | — | | 15.0 | (3.94)^{d} |
| Cₘₐₓ/Dose (μg/mL)/mg | 0.121 | (0.151, 102) | 0.133 | (0.138, 31) |
| AUCₜₐᵤ/Dose (μg · day/mL)/mg | 2.08 | (2.08, 8)^{b} | 2.73 | (2.81, 27) |

AUCₜₐᵤ = AUC limited to the end of one dosing interval following multiple doses, where 28 days was used; AUCₜ = AUC limited to the end of one dosing interval following multiple doses, where actual time was used.
^{a}Median (minimum-maximum)
^{b}N = 6 for C_{trough} AUCₜₐᵤ and AUCₜₐᵤ/Dose for Week 0 (Day 1)
^{c}Harmonic mean (Pseudo SD)
^{d}Terminal half-life after the 3^{rd} 800 mg SC dose on Week 8 (Day 57)

### The Anti-Drug Antibody (ADA) Assessment in PLWH

The treatment-emergent ADA incidence after single or multiple IV doses of mAb1 in PLWH was summarized in Table 4E. The same immunogenicity assay that was previously used in healthy subjects was used to assess the treatment-emergent ADA in PLWH (LLOQ of 10 titer).

In Part A Group 1 and 2, the ADA incidences were 1/5 (20%) and 1/4 (25%), respectively. In Part B Group 3, the ADA incidences were 3/7 (42.9%) and 0/5 (0%) for subjects receiving one or two doses, respectively. In Part B Group 3a and 4, the ADA incidences were 3/10 (30%) and 2/11 (18.2%), respectively. Overall, for both Parts A and B, 10 of 42 subjects (10/42, 23.8%) who received mAb1 were identified as ADA positive.

TABLE 4E
Summary of ADA Incidence After Single Dose or Multiple Doses of mAb1 in Study D

| | | | Aviremic Subjects Part B | | | | |
|---|---|---|---|---|---|---|---|
| | Viremic Subjects Part A | | | 800 mg IV | 800 mg SC | 1600 mg IV | |
| | 800 mg IV | 1600 mg IV | 800 mg IV | Q4W, 2 | Q4W, 3 | Q4W, 3 | All Subjects |
| | Single Dose | Single Dose | 1 Dose | Doses | Doses | Doses | Total |
| | N = 5 | N = 4 | N = 7 | N = 5 | N = 10 | N = 11 | (All Groups) |
| ADA | 1/5 | 1/4 | 3/7 | 0/5 | 3/10 | 2/11 | 10/42 |
| Positive | (20.0%) | (25.0%) | (42.9%) | (0.0%) | (30.0%) | (18.2%) | (23.8%) |
| Subjects | | | | | | | |
| (n/N) | | | | | | | |

ADA = Anti-Drug Antibody;
IV = Intravenous;
Q4W = every 28 days;
SC = Subcutaneous;
N = subjects receiving active drug;
n = number of ADA positive subjects
Note:
Incidence of ADA presented as n/N, where n = ADA positive subjects

α4β7 expression was measured with mAb1 competing antibody. Percentage of α4β7 positive cells were comparable across the study (in both viremic and aviremic subjects in Parts A and B, respectively) on CD4+ and CD8+ T cell subsets (total, naïve or central/transient memory).

The mAb1 achieved immediate saturation of α4β7 integrin receptors on CD4 cells after completion of each single dose infusion and within 2 hours of SC dosing. Nearly complete α4β7 integrin receptor saturation (>90%) was achieved at all dose levels, and the duration of α4β7 receptor saturation was concentration dependent. Duration of nearly complete receptor saturation (>90%) on CD4+ T cells was 42 days and 56 days among subjects receiving a single dose of 800 mg and 1600 mg, respectively, in Part A. Subjects receiving 800 mg IV single dose and 800 mg IV QW4×2 doses in Part B had nearly complete receptor saturation (>90%) on CD4+ T cells for 71-85 days and 99-113 days, respectively. Subjects receiving 1600 mg IV Q4W×3 doses or 800 mg SC QW4×3 doses in Part B had nearly complete receptor saturation (>90%) on CD4+ T cells for 141-225 days. No α4β7 receptor saturation was observed for subjects receiving placebo.

The mAb1-mediated receptor internalization was measured with a non-competing antibody targeting the 37 subunit. A reduction in 37 expression was observed on CD4 and CD8 cells, which indicated mAb1-mediated receptor internalization. The mAb1-mediated 37 internalization was observed at all doses and appeared partial.

### Laboratory Summary

There was no clinically significant trend observed for changes in CD4 T cell count or hematology, chemistry, or urinalysis parameters. No subject had a confirmed or sustained decline in CD4 T cell count. Four subjects who had received active study drug had increases in aspartate aminotransferase (AST) ≥Grade 2 and one of these subjects had a concurrent Grade 2 alanine aminotransferase (ALT) increase. None of these elevations were considered clinically significant as AEs by the investigator. Alternative etiologies included heavy weight-lifting and strenuous exercise (supported by increased CPK levels) and increased alcohol use. Upon stopping those activities, the transaminase levels rapidly improved and normalized. There was no interruption or discontinuation of study drug due to these elevations, and the subjects completed the study without recurrence. Of the cases that met the criteria for review by an independent External Hepatic Committee, none were considered as related to study drug.

mAb1 Safety Summary

mAb1 was well-tolerated as single doses in healthy volunteers and as multiple doses in PLWH in Phase 1 of study D. Overall, there was no discontinuation of study drug due to an AE, no SAE or death, and the frequency of drug-related AEs was low. There was no individual or overall trend in any of the safety parameters, including in viremic PLWH, and those who received multiple dosing of 1600 mg. In PLWH, fatigue was the most common drug-related AE in the active drug groups (8.7%) and occurred at a higher frequency in the placebo group (27.3%) and there was no occurrence of a protocol-defined AESI. In this study, no new AESI or new safety signal was identified, including from 1600 mg IV dosing in viremic and aviremic PLWH.

### Example 5. Study E MAb 2 (Anti-PD-1) and/or MAb 1 (Anti-4P7)

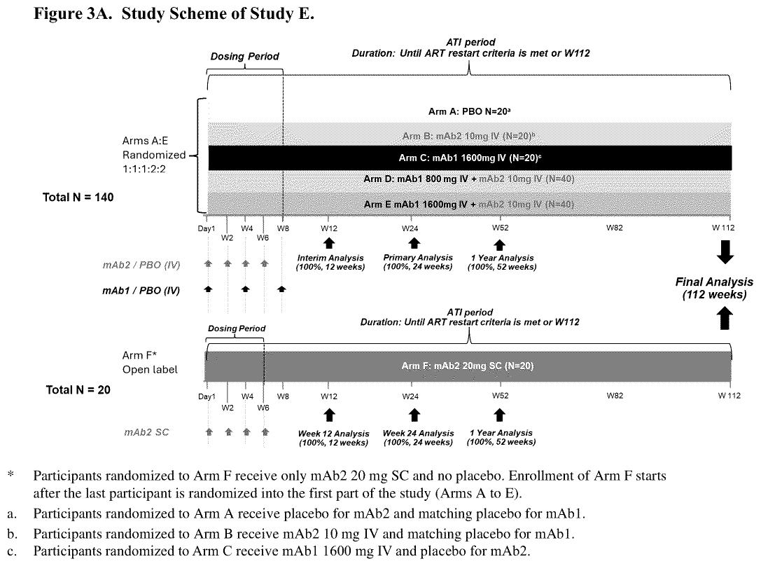

This is a Phase 2, multicenter, randomized, double-blind, placebo-controlled study to evaluate the efficacy, safety, tolerability, and pharmacokinetics of anti-α4β7 monoclonal antibody mAb1 (having heavy chains shown as SEQ ID NO:18 and may also have a C-terminal lysine truncated heavy chain of SEQ ID NO: 21 and light chains shown as SEQ ID NO:19) and/or anti-PD1 monoclonal antibody mAb2 (having heavy chains shown as SEQ ID NO:9 and may also have a C-terminal lysine truncated heavy chain of SEQ ID NO: 20 and light chains shown as SEQ ID NO:10) in chronic PLWH adults that are on stable ART, undergoing anti-retroviral treatment interruption (ATI). This population is selected considering prior stability on ART with demonstrated viral control and sufficient CD4 counts to minimize risks associated with ATI and viral rebound. The study E study scheme is shown in FIG. 3A.

Approximately 160 subjects with a confirmed HIV diagnosis that are on stable ART undergoing ATI are enrolled in this trial.

The screening period in this study is 35 days and may be extended if needed for logistical reasons and upon TA or MD approval.

The total study duration is approximately 117 weeks (inclusive of the screening period).

During the double-blind treatment period, the first enrolled subjects (N=140) are randomized in parallel in a 1:1:1:2:2 randomization ratio to one of the following five treatment arms as shown in Table 5A.

After the first part of the study is fully enrolled, 20 more subjects are allocated to a new arm (Arm F). Participants in Arm F receive mAb2 20 mg SC every 2 weeks (total 4 doses). In Study A, the 20 mg SC single dose of mAb2 demonstrated approximately 62% SC bioavailability. Based on PK simulations, the mAb2 20 mg SC Q2W (total 4 doses) regimen is expected to provide similar AUC_{2 weeks} following the last dose, compared to mAb2 10 mg IV Q2W (total 4 doses) regimen. Therefore, 20 mg SC Q2W was selected for Arm F.

TABLE 5A
Treatment Arms of Example 5 Study E Combination of mAb2
(Anti-PD-1) and/or mAb1 (Anti-α4β7 integrin)

| | |
|---|---|
| Arm A | Placebo: mAb2 placebo IV Q2W for 4 doses on Week 0 (Day 1) and Weeks 2, |
| | 4, and 6 (Days 15, 29, 43) + mAb1 matching placebo IV Q4W for 3 doses on |
| | Week 0 (Day 1) and Weeks 4 and 8 (Days 29 and 57) (n = 20) |
| Arm B | mAb2 10 mg IV: 10 mg IV of mAb2 Q2W for 4 doses on Week 0 (Day 1) and |
| | Weeks 2, 4, and 6 (Days 15, 29, 43) + mAb1 matching placebo IV Q4W for 3 |
| | doses on Week 0 (Day 1) and Weeks 4 and 8 (Days 29 and 57) (n = 20) |
| Arm C | mAb1 1600 mg IV: mAb2 placebo IV Q2W for 4 doses on Week 0 (Day 1) |
| | and Weeks 2, 4, and 6 (Days 15, 29, 43) + mAb1 1600 mg IV Q4W for 3 |
| | doses on Week 0 (Day 1) and Weeks 4 and 8 (Days 29 and 57) (n = 20) |
| Arm D | mAb2 10 mg IV + mAb1 800 mg IV: mAb2 IV 10 mg Q2W for 4 doses on |
| | Week 0 (Day 1) and Weeks 2, 4, and 6 (Days 15, 29, 43), combined with |
| | mAb1 800 mg IV Q4W for 3 doses on Day 1 and Weeks 4 and 8 (Days 29 and |
| | 57) (n = 40) |
| Arm E | mAb2 10 mg IV + mAb1 1600 mg IV: mAb2 IV 10 mg Q2W for 4 doses on |
| | Week 0 (Day 1) and Weeks 2, 4, and 6 (Days 15, 29, 43), combined with |
| | mAb1 1600 mg IV Q4W for 3 doses on Week 0 (Day 1) and Weeks 4 and 8 |
| | (Days 29 and 57) (n = 40) |
| Arm F (open label - | mAb2 20 mg SC Q2W for 4 doses on Week 0 (Day 1) and Weeks 2, 4 and 6 |
| no placebo) | (Days 15, 29, 43) alone (n = 20) |

On Week 0 (Day 1) and Week 4 (Day 29) for Arms A to E, when mAb2 and mAb1 are administered on the same day, mAb2 is administered prior to mAb1 as its safety profile is well characterized in oncology populations. A monitoring interval is planned between completion of mAb2 administration and start of mAb1 administration, as well as after mAb1 administration.

Starting on Week 0 (Day 1) (following the first study drug dose), subjects undergo an intensively monitored ART interruption (ATI) planned for 112 weeks or until ART restart criteria is met.

### Study Objectives

Objectives of this study include evaluating efficacy, safety, tolerability, and pharmacokinetics of mAb2 and/or mAb1 versus placebo in chronic PLWH adults stable on ART submitted to an ATI.

### Study Endpoints:

An endpoint of this study is to assess the proportion of subjects with viral control (VL <1000 copies/mL) at week 24 that have not restarted ART. Other endpoints are to assess peak viral load at rebound, prior to restarting ART, the time to viral rebound, defined as when viral load >1000 copies/mL during ART interruption.

The safety end points to be examined include drug-related Grade 3 or higher adverse events (AEs), frequency and severity of adverse events (AEs) and frequency of adverse events of special interest (AESIs: IRAEs, infusion-related reactions, injection-site-reactions and hepatotoxicity), RRS during the ATI period, and virologic failure following ART restart.

Pharmacokinetic endpoints include assessing both monoclonal antibodies following the first and last doses for the maximum concentration (Cₘₐₓ), time to maximum concentration (Tₘₐₓ), evaluating the area under the concentration-time curve during the dosing interval (AUCₜₐᵤ) after the first and the last doses and finally measuring the terminal half-life (t_{1/2}) after the final dose.

The pharmacodynamic endpoints evaluate engagement of the monoclonal antibodies with their targets. These include assessing PD-1 receptor expression and saturation (%) on peripheral CD4+ and CD8+ T cell subsets, examining α4β7 integrin expression and saturation (%) on peripheral CD4+ and CD8+ T cell subsets, and quantifying peripheral 07+ expression and longitudinal changes on CD4+ and CD8+ T cell subsets.

Other study endpoints examine viral control (viral load <50, 200 or 1000 copies/mL) at weeks 12, 24, 52 and 112 without ART restart, area under the HIV RNA curve, time to ART restart, time to viral rebound (first viral load >1000 copies/mL during ART interruption), achievement of ART restart criteria prior to Week 12 of ART interruption. Time to viral re-suppression after ART restart, and changes in patient reported outcomes (PRO) measurements from baseline at weeks 8, 24, 52 and 112.

### ART Restart Criteria:

Specific criteria for re-initiation of ART (at least one of the following):
1. Increased Viral Load:
Confirmed HIV-1 viral load (VL) >100,000 copies/mL (defined as two consecutive measurements). Confirmatory testing for the VL criteria should be completed as soon as possible.
HIV-1 VL >10,000 copies/mL (confirmed over the period of 4 weeks)
HIV-1 VL >1,000 copies/mL (confirmed over the period of 6 weeks)
Confirmed absolute CD4 count <350 cells/μL (defined as 2 consecutive measurements). Confirmatory testing for the CD4 criteria should be completed as soon as possible.
2. Pregnancy
3. Grade 3 or higher Retroviral rebound syndrome.
4. Any adverse event which, in the judgment of the investigator, presents a substantial clinical risk to the subject to continued ATI.
5. Any social or behavioral condition which, in the judgment of the investigator, presents a substantial clinical risk to the participant to continue ATI, including participant's request.

### Study Goals

Efficacy of the combination of mAb2 and mAb1, as defined by primary and secondary efficacy analyses, is evaluated.

### Eligibility Criteria

1. Adults of any sex and gender with a condition of generally good health, 18 to 70 years of age, body mass index ≥18.0 to <35.0 kg/m².
2. HIV-1 infected on ART for at least 12 months prior to screening and on current ART regimen for at least 8 weeks prior to screening (current ART regimen cannot include a non-nucleoside reverse transcriptase inhibitor [NNRTI]).
3. Subject must not have had prior exposure to long-acting antiretrovirals within 24 weeks or within a period defined by 5 half-lives, whichever is longer, prior to the first dose of study drug.
4. Subject must have negative HIV Ab at screening.
5. Plasma HIV-1 RNA below lower limit of quantification (LLOQ) at screening and for at least 6 months prior to screening.
6. CD4+ T cell count ≥500 cells/μL at screening and no known evidence of CD4 <500 cells/mm³ in the last 12 months prior to screening.
7. After receiving counseling about risks of ATI, including risk of HIV transmission, must be willing to undergo ART interruption and stay off ART for the all period if ART criteria is not met.
8. Must agree to use an effective barrier method of protection (male and/or female condoms) during sexual activity for protection against HIV transmission throughout the study.
9. Subject must have history of CD4+ T cell nadir of ≥200 cells/μL during chronic infection.
10. Site investigator anticipates that a fully active alternative ART regimen could be constructed and would be available in the event of virologic failure on the subject's current ART regimen.
11. Subject must not have a history of AIDS-defining illness.
12. Subject must not have evidence of initiation of ART <3 months after diagnosis (acute HIV-1 infection).
13. Subject must not have active or suspected malignancy or history of malignancy (other than basal cell skin cancer or cervical carcinoma in situ).
14. Subject must not have history of or active immunodeficiency (other than HIV).
15. Subject must not have active autoimmune disease or history of autoimmune disease that has required systemic treatment.
16. Subject must not have prior receipt of immunomodulatory or immunosuppressive (including IV/PO steroids at any dose, but excluding steroids that are inhaled, topical or via local injection) therapy within 6 months prior to the first dose of study drug.
17. Subject must not have had prior therapy/exposure to mAb2 or any other immune checkpoint inhibitor (e.g., anti-PD-1, anti-PD-L1, anti-PD-L2, anti-CTLA-4) or mAb1 or any monoclonal antibody targeting α4β7 or 37 integrins (e.g., vedolizumab).
18. Subject must not be in current treatment with immunoglobulin therapy.
19. Subject must not have prior exposure to monoclonal antibody-based immunotherapy or previous enrollment in antibody-based immunotherapy clinical trials within 24 weeks or within a period defined as 5 half-lives, whichever is longer, prior to the first dose of study drug.
20. Subject must not have participated in ATI study or any HIV-cure based (also including HIV broadly neutralizing monoclonal antibodies) investigational study in the past 48 weeks, prior to the first dose of study drug.
21. Subject must not have current hepatitis B virus or hepatitis C virus infection.
22. Subject must not have clinically significant medical disorders that might expose the subjects to undue risk of harm, confound study outcomes, or prevent the subject from completing the study (including but not limited to significant or unstable cardiac, neurologic or pulmonary disease, chronic active infectious disease except for HIV, chronic liver disease, poorly controlled diabetes mellitus and history of Stevens Johnson Syndrome, toxic epidermal necrolysis (TEN), or drug reaction with eosinophilia and systemic symptoms (DRESS)).
23. Subject must not have known psychiatric or substance abuse disorders that would interfere with adherence to study requirements. Subjects with recent substance abuse or alcohol abuse history and/or with positive urine screen for drugs of abuse and alcohol at screening may be included after evaluation by the investigator that the use would not interfere with adherence to study requirements.
24. Subject must not be pregnant, breastfeeding, or considering becoming pregnant during the study.
25. Subject must not have had a live vaccine administered 4 weeks prior to first dose.

### Example 6 Anti-PD-1 Monoclonal Antibody PK Models and Simulations

As demonstrated in Study B, the suppression of viral load could be achieved following administration of mAb2 10 mg IV Q2W (for 4 doses), where the 1^{st} dose was given at the beginning of analytical treatment interruption. Based on the study results shown in the other examples, mAb2 10 mg IV Q2W regimen could saturate PD-1 receptor of peripheral total CD8+ T cells for at least 70 days, which subsequently triggers immune-mediated viral load control.

In general, the drugs within the same class as mAb2 (i.e., anti-PD-1 monoclonal antibody), including but not limited to, Nivolumab, Pembrolizumab, and Cemiplimab, may potentially trigger immune-mediated viral load control if the selected dosing regimens are able to saturate PD-1 receptor of peripheral total CD8+ T cells for at least 70 days post-analytical treatment interruption.

To demonstrate the minimal potential efficacious dosing regimen of Nivolumab, Pembrolizumab, and Cemiplimab to trigger immune-mediated viral load control following IV or SC administrations, the population PK modeling and simulation approach was used. The population PK modeling and simulation approach is a well-known model-based approach to identify potential efficacious dosing regimens. In vitro PD-1 receptor saturation assays were performed to identify EC95 concentrations of mAb2, Nivolumab, Pembrolizumab, and Cemiplimab. The estimated EC95 serves as target concentrations in the simulations' scenarios.

mAb2

The preliminary mAb2 population pharmacokinetic (PK) model was developed for people living with HIV (PLWH) following IV or SC administration based on data collected from Phase 1b Study A and B. Preliminary estimates of the PK parameters and their associated variability are shown in Table 6A. To date, no covariates were tested due to the limited population size (N=53) in the Phase 1b Study A and B. The developed mAb2 population PK model is able to capture the central tendency of PK data seen in Studies A and B and is robust in predicting PK profiles for PLWH who undergo analytical treatment interruption.

TABLE 6A
Preliminary Parameter Estimates and Variability
of mAb2 Population PK Model in People Living
with HIV Following IV or SC Administration

| | Population | | 95% Confidence |
|---|---|---|---|
| Parameter | Estimate | % RSE | Interval |
| CL (L/day) | 0.241 | 4.64 | (0.220, 0.263) |
| V_{c} (L) | 3.29 | 6.75 | (2.89, 3.76) |
| Vₘₐₓ (mg/day) | 0.0784 | 20.8 | (0.0523, 0.117) |
| Vₚ (L) | 1.38 | 11.7 | (1.10, 1.73) |
| Q (L/day) | 0.840 | 22.4 | (0.544, 1.30) |
| Kₘ (mcg/mL) | 0.0774 | 32.8 | (0.0413, 0.145) |
| F1 for SC model | 0.480 | 11.6 | (0.383, 0.603) |
| Kₐ (1/day) for SC model | 0.237 | 17.6 | (0.168, 0.334) |
| Prop. error | 0.0559 | 5.33 | (0.0501, 0.0618) |
| Prop. error for SC model | 0.185 | 19.2 | (0.116, 0.255) |

| | Population | | % Shrinkage |
|---|---|---|---|
| Parameter | Estimate | % CV | (SC model) |
| IIV on CL | 0.211 | 48.5 | 7.28 (40.0) |
| IIV on V_{c} | 0.117 | 35.2 | 1.72 (35.5) |
| IIV on Vₘₐₓ | 0.450 | 75.3 | 16.5 (25.7) |
| IIV on Kₐ for SC model | 0.0906 | 30.8 | 31.3 |

CL = clearance; V_{c} = central volume of distribution; Vₘₐₓ = maximal reaction rate; Vₚ = peripheral volume of distribution; Q = inter-compartmental clearance; Kₘ = Michaelis-Menten constant; F1 = bioavailability; Kₐ = absorption rate constant; IIV = inter-individual variability; % RSE was calculated as the standard error of the estimate divided by the absolute value of the mean of the estimate multiplied by 100. % CV was calculated as SQRT(exp(ω²) − 1)*100).

### Nivolumab

Nivolumab is a fully human IgG4 monoclonal antibody that selectively binds to the programmed death-1 (PD-1) membrane receptor, inhibiting the interaction between PD-1 and its ligands and promoting antitumor immune responses (Zhao et al. Clin Pharmacol Ther, 2024:115: 488-497). The nivolumab population pharmacokinetic model developed for subjects with unresectable or metastatic melanoma following IV administration is publicly available (Opdivo® (nivolumab) Clinical Pharmacology and Biopharmaceutics Review(s), BLA 125554Origs000. Available from World Wide Web at accessdata.fda.gov/drugsatfda_docs/nda/2014/125554orig1s000clinpharmr.pdf).

Estimates of the pharmacokinetic parameters and their associated variability are shown in Table 6B. It is noted that effects of oncology related covariate, namely Eastern Cooperative Oncology Group (ECOG) performance (reference=0), is not presented in the parameter table, as it is not relevant to people living with HIV, the population of interest.

TABLE 6B
Parameter Estimates and Variability of Nivolumab Population
PK Model in Subjects with Unresectable or Metastatic
Melanoma Following IV Administration

| | Population | 95% Confidence |
|---|---|---|
| Parameter | Estimate | Interval |
| CL (L/day) | 0.208 | (0.198, 0.217) |
| V_{c} (L) | 3.87 | (3.75, 3.99) |
| Q (L/day) | 0.710 | (0.641, 0.790) |
| Vₚ (L) | 3.80 | (3.60, 4.04) |
| Exponent for the Effect of Body | 0.700 | (0.576, 0.809) |
| Weight on CL (Ref = 80 kg) | | |
| Exponent for the Effect of eGFR on | 0.172 | (0.0670, 0.276) |
| CL (Ref = 80 mL/min/1.73 m2) | | |
| Exponent for the Effect of Body | 0.534 | (0.463, 0.607) |
| Weight on V_{c} (Ref = 80 kg) | | |
| V_{c} Factor for Male Compared to | 0.130 | (0.0937, 0.167) |
| Female | | |
| Proportional Error | 0.207 | (0.197, 0.218) |

| | Population | |
|---|---|---|
| Parameter | Estimate | % Shrinkage |
| IIV on CL | 0.188 | 9.92 |
| IIV on V_{c} | 0.0488 | 17.8 |
| IIV on CL and V_{c} | 0.0438 | — |
| IIV on Vₚ | 0.294 | 28.5 |

CL = clearance;
V_{c} = central volume of distribution;
Q = inter-compartmental clearance;
Vₚ = peripheral volume of distribution;
Ref = reference;
eGFR = estimated glomerular filtration rate calculated using CKD-EPI equation;
IIV = inter-individual variability;
CL = 0.208 × (WTKG/80)^{0.700} × (eGFR/80)^{0.172};
V_{c} = 3.87 × (WTKG/80)^{0.534} × [exp(0.130) if male];
WTKG = body weight

The nivolumab population pharmacokinetic model developed for subjects with advanced solid tumors following SC administration is publicly available (Zhao et al. Clin Pharmacol Ther, 2024:115: 488-497). Estimates of the pharmacokinetic parameters and their associated variability are shown in Table 6C. Effects of oncology related covariates, such as performance status (reference=0) or cancer types, are not presented in the parameter table, as they are not relevant to people living with HIV.

TABLE 6C
Parameter Estimates and Variability of Nivolumab Population PK Model
in Subjects with Advanced Solid Tumors Following SC Administration

| Parameter | Population Estimate | 95% Confidence Interval |
|---|---|---|
| CL (L/day) | 0.259 | (0.242, 0.274) |
| V_{c} (L) | 4.27 | (4.19, 4.34) |
| Q (L/day) | 0.782 | (0.684, 0.881) |
| Vₚ (L) | 2.63 | (2.43, 2.83) |
| Emax on CL | −0.303 | (−0.371, −0.235) |
| T50 (day) | 59.2 | (53.3, 65.4) |
| Hill Factor | 2.97 | (1.82, 4.12) |
| Kₐ (1/day) | 0.396 | (0.322, 0.468) |
| F1 | 0.767 | (0.688, 0.845) |
| Exponent for the Effect of Body Weight on CL | 0.588 | (0.526, 0.651) |
| (Ref = 80 kg) | | |
| Exponent for the Effect of Body Weight on Vc | 0.626 | (0.556, 0.695) |
| (Ref = 80 kg) | | |
| Exponent for the Effect of eGFR on CL | 0.142 | (0.0966, 0.188) |
| (Ref = 90 mL/min/1.73 m2) | | |
| CL Factor for Female Compared to Male | −0.162 | (−0.194, −0.130) |
| V_{c} Factor for Female Compared to Male | −0.139 | (−0.173, −0.104) |
| Kₐ Factor for Female Compared to Male | −0.235 | (−0.435, −0.0358) |
| Kₐ Factor for No-Coadministration Compared | −0.625 | (−0.830, −0.420) |
| to Coadministration with rHuPH20 | | |
| F1 Factor for Female Compared to Male | 0.842 | (0.720, 0.964) |
| F1 Factor for No-Coadministration Compared | 1.01 | (0.891, 1.13) |
| to Coadministration with rHuPH20 | | |
| Proportional Error | 0.204 | (0.194, 0.214) |
| IIV on CL | 0.113 | (0.102, 0.125) |
| IIV on V_{c} | 0.126 | (0.0990, 0.153) |
| IIV on CL and V_{c} | 0.0364 | (0.0289, 0.0439) |
| IIV Vₚ | 0.225 | (0.179, 0.271) |
| IIV on Eₘₐₓ | 0.0516 | (0.0353, 0.0679) |
| IIV on Kₐ | 0.0946 | (0.0414, 0.148) |
| IIV on F1 | 0.462 | (0.221, 0.703) |
| IIV on Kₐ and F1 | 0.177 | (0.0863, 0.269) |

CL = clearance;
V_{c} = central volume of distribution;
Q = inter-compartmental clearance;
Vₚ = peripheral volume of distribution;
Eₘₐₓ = estimate of the maximal change in CL;
T₅₀ = time, at which the change in CL is 50% of Eₘₐₓ;
Kₐ = absorption rate constant;
F1 = bioavailability;
Ref = reference;
eGFR = estimated glomerular filtration rate calculated using CKD-EPI equation;
IIV = inter-individual variability;
CL = 0.259 × exp(Eₘₐₓ × time^{Hill}/(T₅₀^{Hill} + time^{Hill})) × (WTKG/80)^{0.588} × (eGFR/90)^{0.142} × [exp(−0.162) if female];
V_{c} = 4.27 × (WTKG/80)^{0.626} × [exp(−0.139) if female];
Kₐ = 0.396 × [exp(−0.235) if female] × [exp(−0.625) if no-coadministration];
F1_{Ref} = 0.767 × [exp(0.842) if female] × [exp(1.01) if no-coadministration];
F1 = exp(ln(F1_{Ref}/(1 − F1_{Ref})))/(1 + exp(ln(F1_{Ref}/(1 − F1_{Ref}))));
WTKG = body weight

### Pembrolizumab

Pembrolizumab is a fully human IgG4 monoclonal antibody that selectively binds to the programmed death 1 (PD-1) membrane receptor, inhibiting the interaction between PD-1 and its ligands and promoting antitumor immune responses. Estimates of the pharmacokinetic parameters and their associated variability based on the final IV pembrolizumab model in subjects with unresectable or metastatic melanoma are shown in Table 6D (Keytruda™ (pembrolizumab) Clinical Pharmacology and Biopharmaceutics Review(s), BLA 125514Orig1s000. Available from World Wide Web at accessdata.fda.gov/drugsatfda_docs/nda/2014/125514Origs000ClinPharnR.pdf). Effect of IgG on pembrolizumab clearance is not presented in the parameter table, as IgG data were not available from the Phase 1b Study B and Study A.

TABLE 6D
Parameter Estimates and Variability of Pembrolizumab
Population PK Model in Subjects with Unresectable
or Metastatic Melanoma Following IV Administration

| Parameter | Population Estimate | % RSE |
|---|---|---|
| CL (L/day) | 0.218 | 2.64 |
| V_{c} (L) | 3.68 | 1.91 |
| Q (L/day) | 0.897 | 13.2 |
| Vₚ (L) | 3.91 | 5.91 |
| Exponent for the Effect of Body | 0.589 | 14.8 |
| Weight on CL and Q | | |
| (Ref = 76.8 kg) | | |
| Exponent for the Effect of Body | 0.474 | 13.4 |
| Weight on V_{c} and Vₚ | | |
| (Ref = 76.8 kg) | | |
| Exponent for the Effect of Albumin | −1.06 | 10.9 |
| on CL (Ref = 39.6 g/L) | | |
| CL Factor for Female Compared to | −0.133 | 28.3 |
| Male | | |
| V_{c} Factor for Female Compared to | −0.142 | 20.8 |
| Male | | |

| Parameter | Population Estimate | % Shrinkage |
|---|---|---|
| IIV on CL and Q | 0.0760 | 14.9 |
| IIV on V_{c} and Vₚ | 0.0180 | 29.7 |

CL = clearance;
V_{c} = central volume of distribution;
Q = inter-compartmental clearance;
Vₚ = peripheral volume of distribution;
Ref = reference;
IIV = inter-individual variability;
RSE = relative standard error;
CL = 0.218 × (WTKG/76.8)^{0.589} × (ALB/39.6)^{(-1.06)} × [(1 − 0.133) if female];
V_{c} = 3.68 × (WTKG/76.8)^{0.474} × [(1 − 0.142) if female];
Q = 0.897 × (WTKG/76.8)^{0.589};
Vₚ = 3.91 × (WTKG/76.8)^{0.474};
WTKG = body weight;
ALB = albumin;
Note:
Since reference values were not available in the Biologics License Applications (BLA) review (Keytruda ™ (pembrolizumab) Clinical Pharmacology and Biopharmaceutics Review(s), BLA 125514Orig1s000. Available from World Wide Web at accessdata.fda.gov/drugsatfda_docs/nda/2014/125514Orig1s000ClinPharmR.pdf), they are taken from the publication (Ahamadi et al. CPT Pharmacometrics Syst Pharmacol. 2017 January; 6(1): 49-57).

### Cemiplimab

Cemiplimab is a recombinant human IgG4 monoclonal antibody that binds to PD-1 and blocks its interaction with PD-L1 and PD-L2. Cemiplimab is an antibody immunotherapy that stimulates an anti-cancer response via programmed cell death protein-1 (PD-1) blockade (Lee et al. Drugs. 2020; 80:813-819). The cemiplimab population PK model in subjects with metastatic or locally advanced cutaneous squamous cell carcinoma following IV administration is publicly available (LIBTAYO™ (Cemiplimab) Multidiscipline Review, BLA 761097Orig1s000. Available from World Wide Web at accessdata.fda.gov/drugsatfda_docs/nda/2018/761097Orig1s000MultidisciplineR.pdf). Estimates of the pharmacokinetic parameters and their associated variability are shown in Table 6E. Effects of IgG and race are not presented in the parameter table.

TABLE 6E
Parameter Estimates and Variability of Cemiplimab Population
PK Model in Subjects with Metastatic or Locally Advanced Cutaneous
Squamous Cell Carcinoma Following IV Administration

| Parameter | Population Estimate | 95% Confidence Interval |
|---|---|---|
| CL (L/day) | 0.287 | (0.274, 0.309) |
| V_{c} (L) | 3.34 | (3.28, 3.40) |
| Q (L/day) | 0.647 | (0.579, 0.722) |
| Vₚ (L) | 1.69 | (1.53, 1.85) |
| Eₘₐₓ on CL | −0.382 | (−0.476, −0.324) |
| T₅₀ (day) | 32.1 | (24.0, 38.6) |
| Hill Factor | 3.17 | (2.33, 4.13) |
| Exponent for the Effect of Body | 0.454 | (0.300, 0.609) |
| Weight on CL and Q (Ref = 75 kg) | | |
| Exponent for the Effect of Body | 0.935 | (0.779, 1.08) |
| Weight on V_{c} and Vₚ (Ref = 75 kg) | | |
| Exponent for the Effect of Alanine | −0.0818 | (−0.137, −0.0240) |
| Aminotransferase on CL and Q | | |
| (Ref = 21 IU/L) | | |
| Exponent for the Effect of Albumin | −1.00 | (−1.23, −0.722) |
| on CL and Q (Ref = 38 g/L) | | |
| Exponent for the Effect of BMI on | −0.553 | (−0.707, −0.378) |
| V_{c} and Vₚ (Ref = 27.1 kg/m2) | | |
| Proportional Error | 0.180 | (0.173, 0.187) |
| Additive Error | 1.34 | (0.0245, 1.95) |
| IIV on CL and Q | 0.0893 | (0.0655, 0.120) |
| IIV on V_{c} and Vₚ | 0.0412 | (0.0345, 0.0484) |
| IIV between CL/Q and V_{c}/Vₚ | 0.0403 | (0.0323, 0.0502) |
| IIV Eₘₐₓ | 0.260 | (0.159, 0.357) |
| IIV on T₅₀ | 0.583 | (0.394, 0.970) |

CL = clearance;
V_{c} = central volume of distribution;
Q = inter-compartmental clearance;
Vₚ = peripheral volume of distribution;
Eₘₐₓ = estimate of the maximal change in CL;
T₅₀ = time, at which the change in CL is 50% of Eₘₐₓ;
Ref = reference;
IIV = inter-individual variability;
CL = 0.287 × exp(Eₘₐₓ × time^{Hill}/(T₅₀^{Hill} + time^{Hill})) × (WTKG/75)^{0.454} × (ALT/21)^{(-0.0818)} × (ALB/38)⁽⁻¹⁾;
V_{c} = 3.34 × (WTKG/75)^{0.935} × (BMI/27.1)^{(-0.553)};
Q = 0.647 × (WTKG/75)^{0.454} × (ALT/21)^{(-0.0818)} × (ALB/38)⁽⁻¹⁾;
Vₚ = 1.69 × (WTKG/75)^{0.935} × (BMI/27.1)^{(-0.553)};
WTKG = body weight;
ALT = alanine aminotransferase;
ALB = albumin

In Vitro Estimation of PD-1 Receptor Saturation on Total CD8+ T Cell Population for mAb2, Nivolumab, Pembrolizumab and Cemiplimab

PD-1 receptor saturation by mAb2, nivolumab (Catalog No. A2002, Selleckchem), pembrolizumab (Catalog No. A2005, Selleckchem) and cemiplimab (Catalog No. A2022, Selleckchem) were assessed in whole blood from 4 donors. All antibodies were tested at concentrations ranging from 10 mcg/mL to 0.005 mcg/mL and incubated with whole blood for 2 hours at room temperature. A flow cytometry assay assessed PD-1 receptor saturation on CD4+ and CD8+ T cells and memory T cell subtypes using a panel consisting of CD3-FITC (clone SK7, Biolegend), PD-1-PE (clone EH12.1, BD Biosciences), CD4-PERCP5.5 (clone RPA-T4, Biolegend), CD8-Alexa700 (clone SK1, Biolegend), CD62L-BUV395 (clone SK11, BD Biosciences), CD45RA-BUV563 (clone HI100, BD Biosciences), live/dead-BV510 (Invitrogen). All analyses were conducted using OMIQ software from Dotmatics.

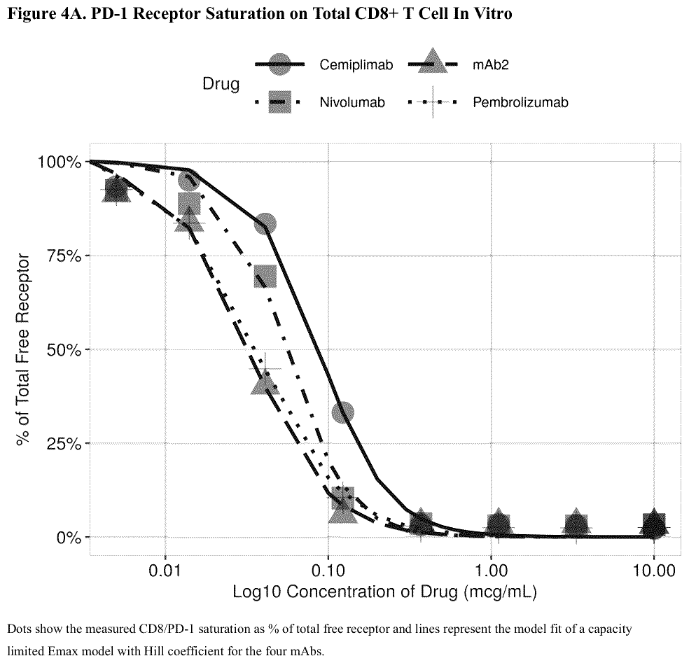

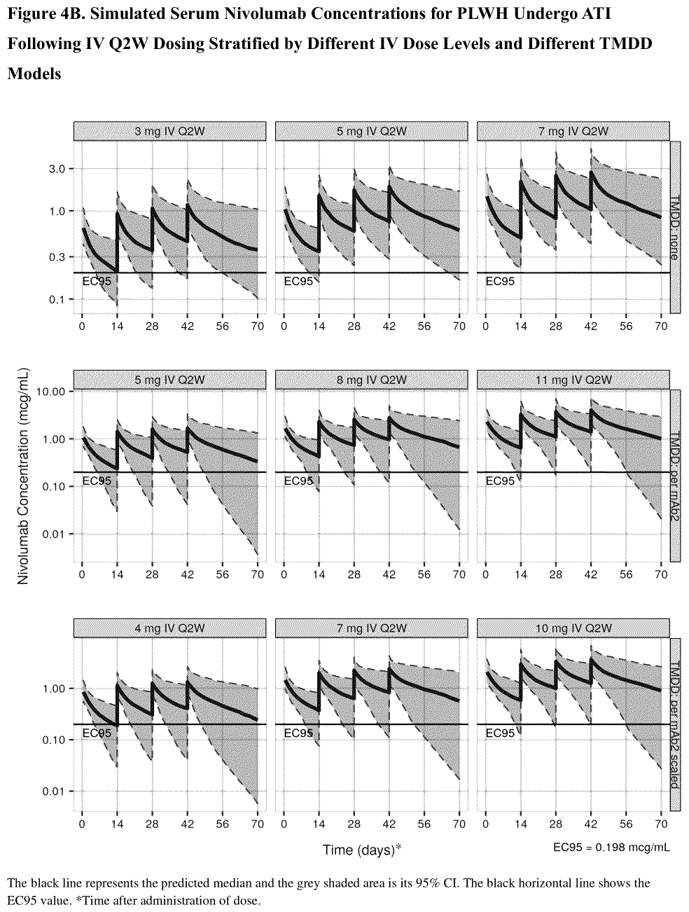

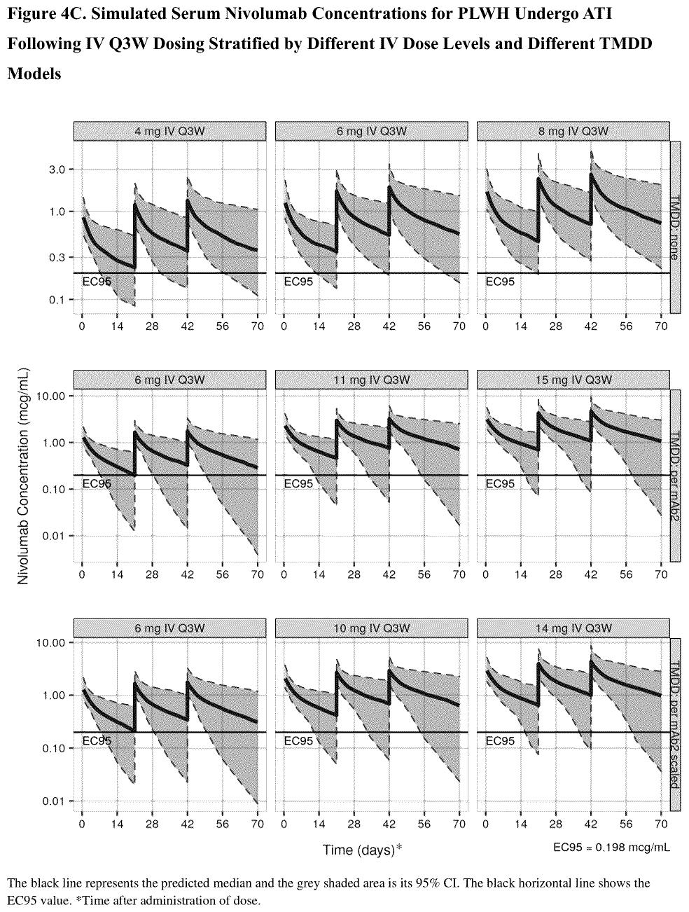

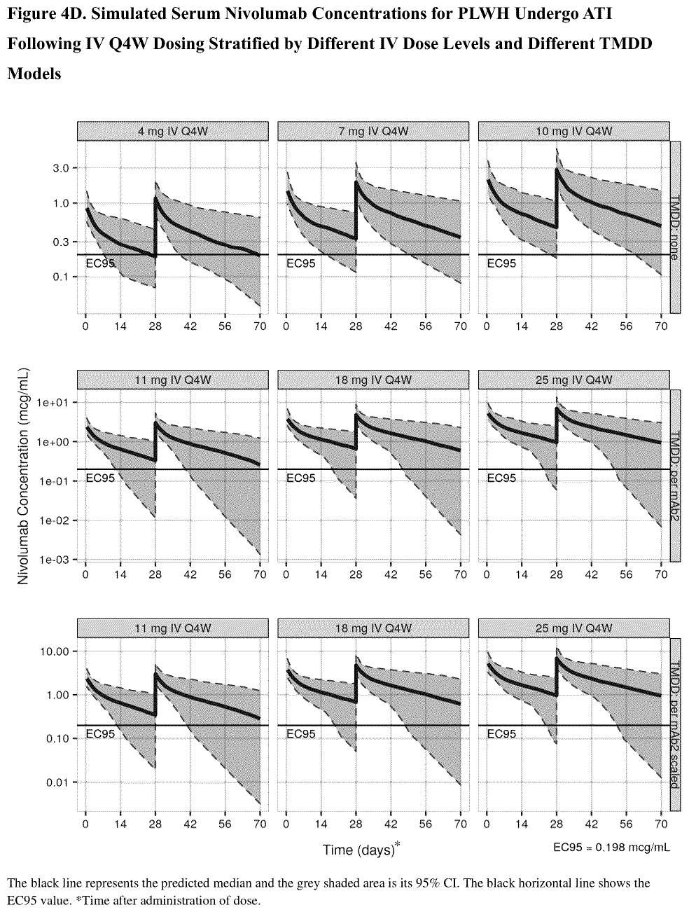

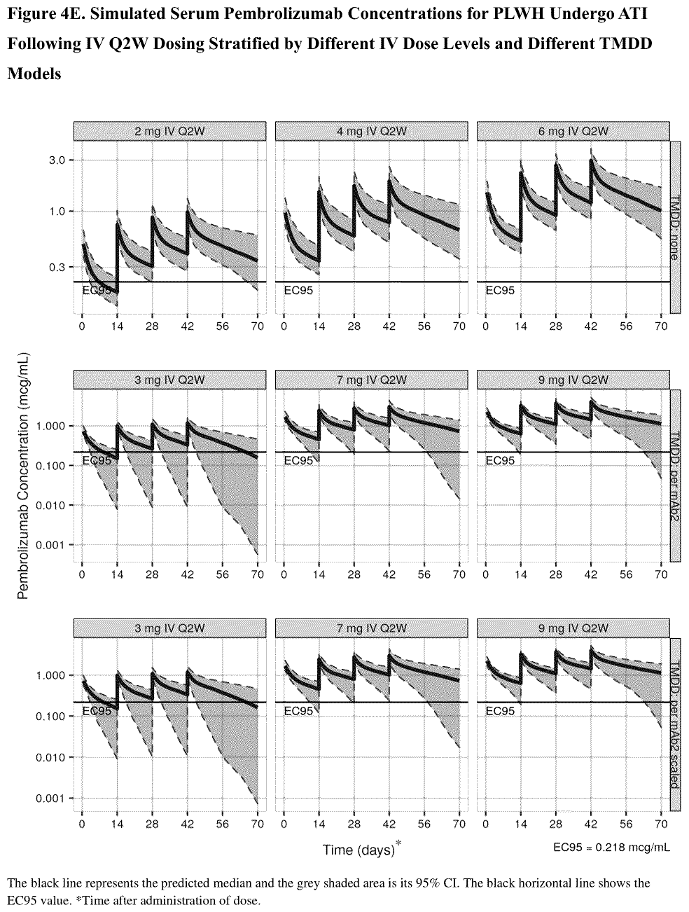

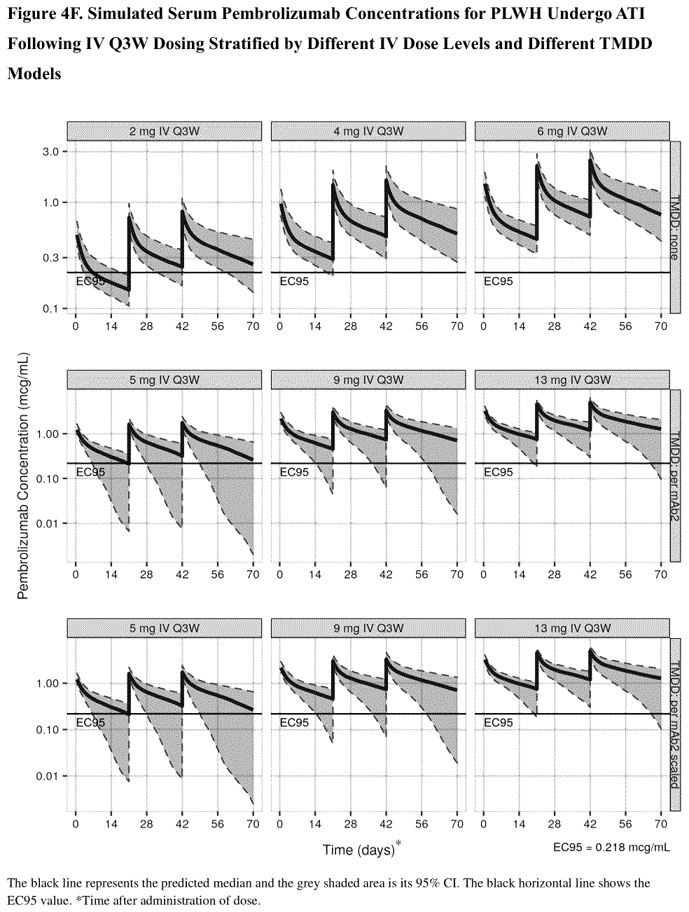

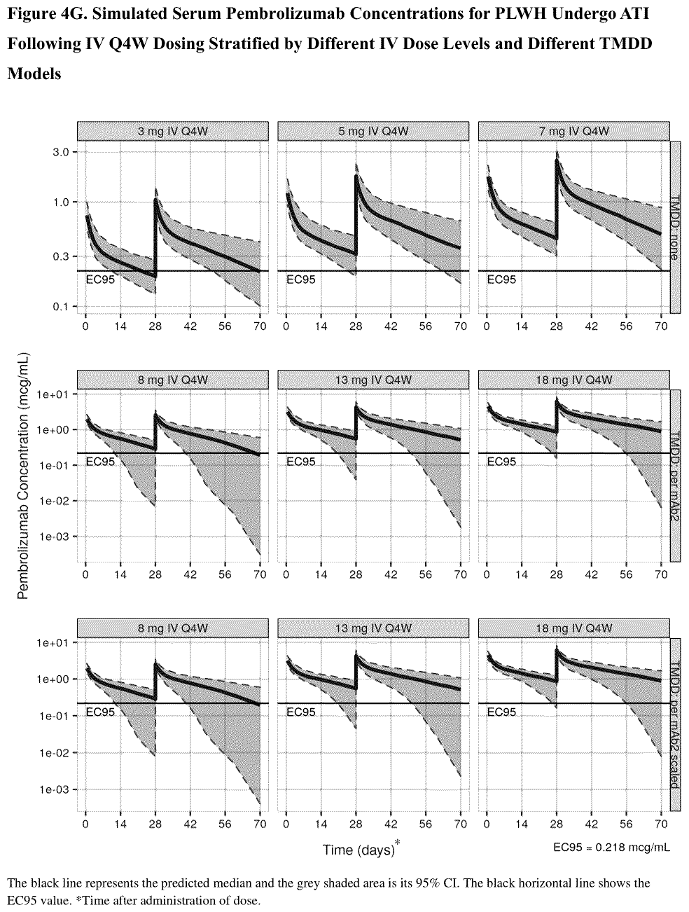

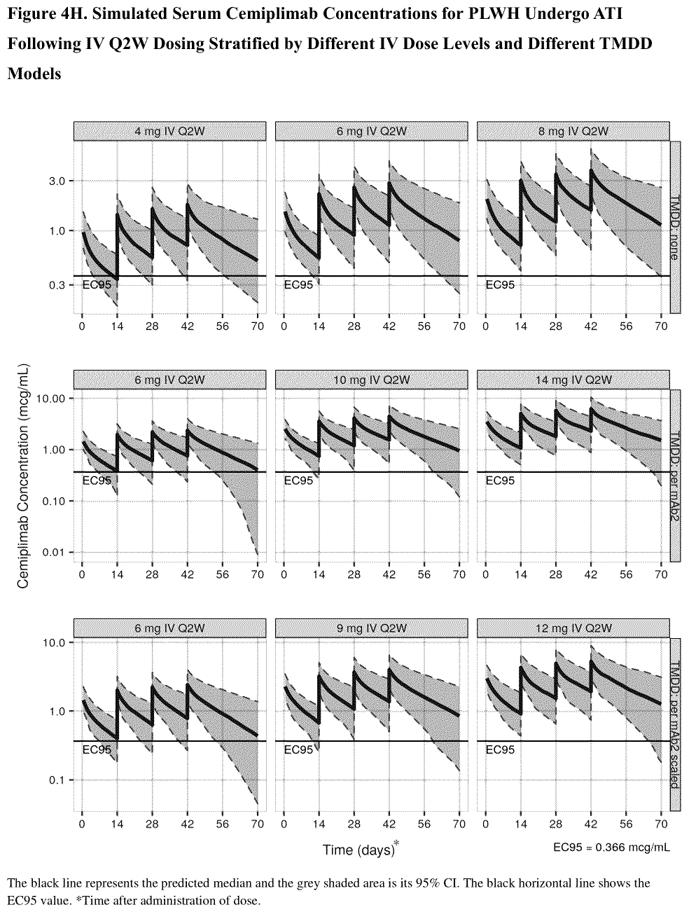

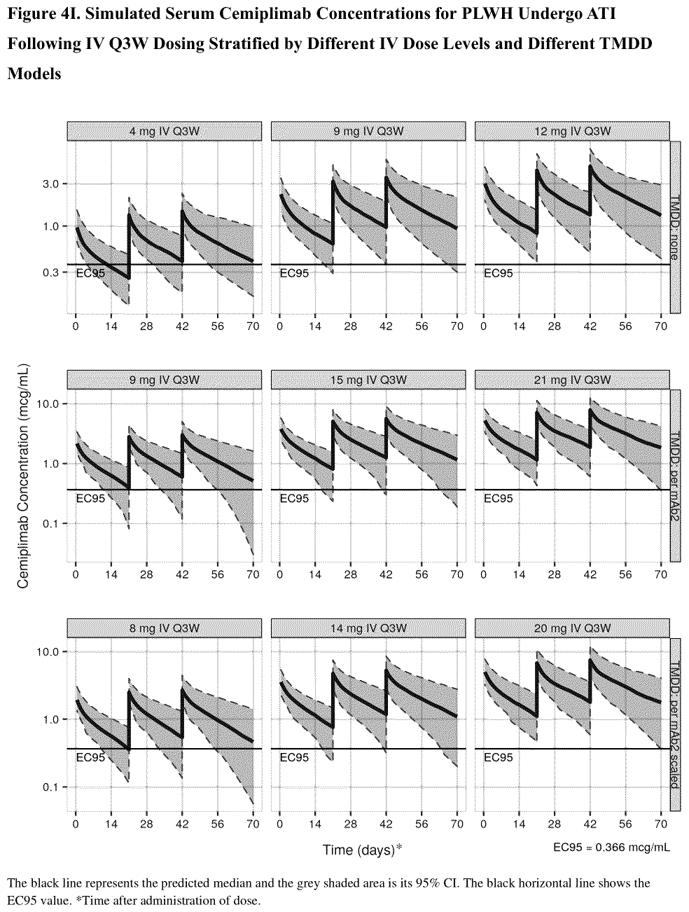

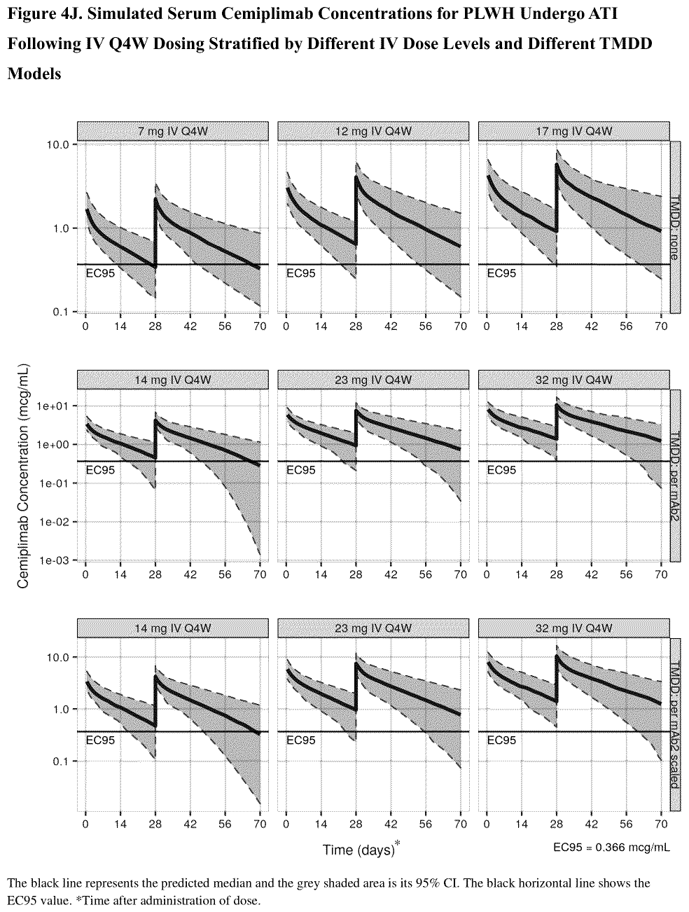

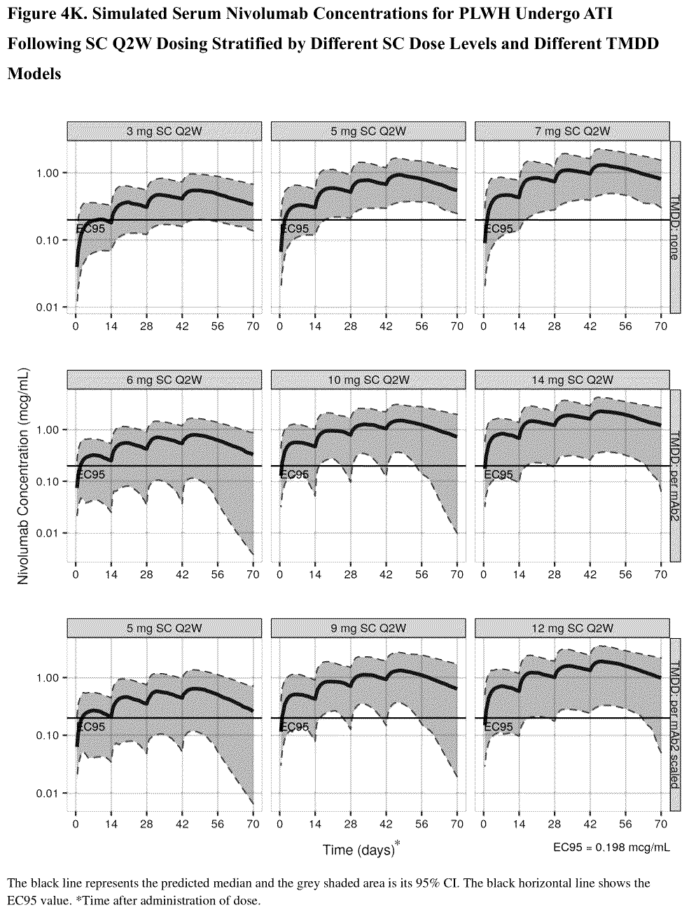

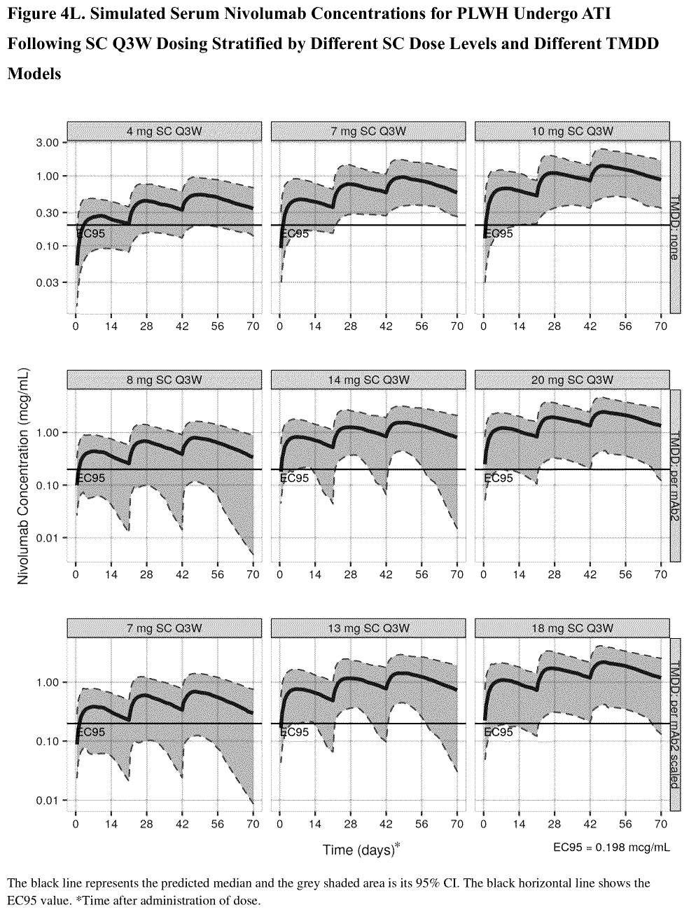

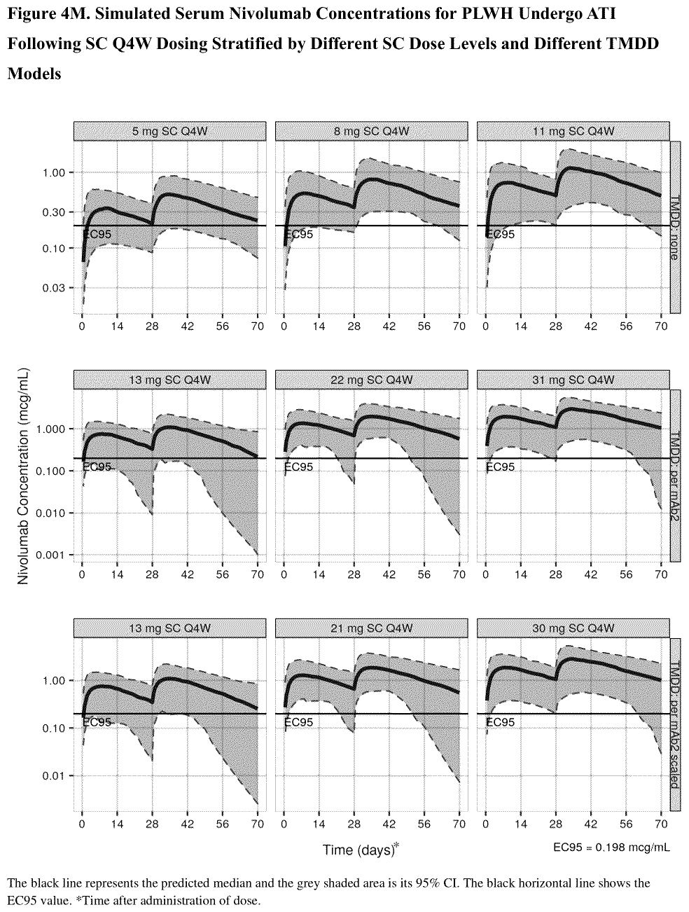

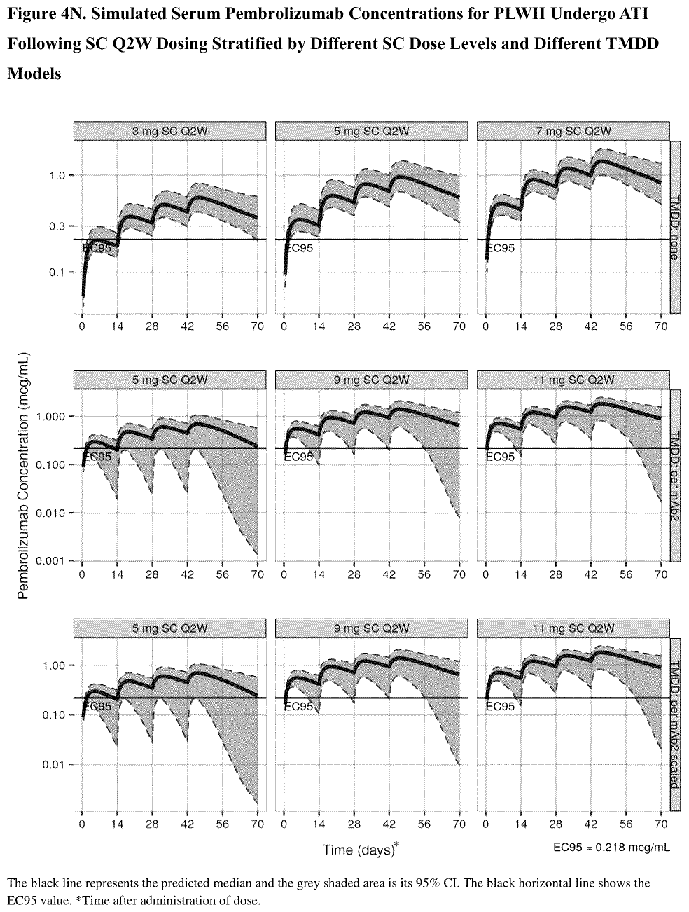

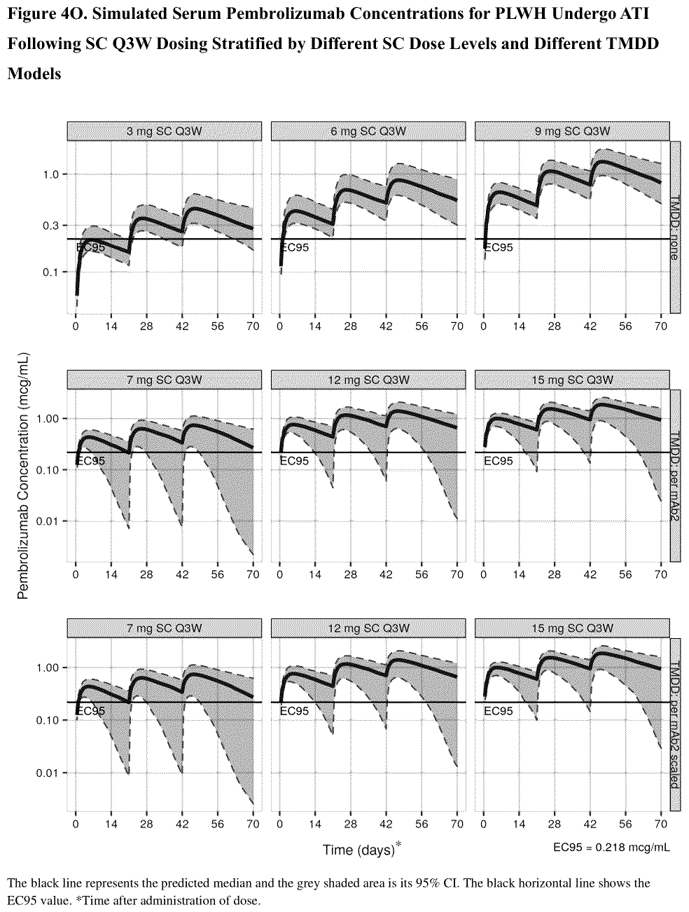

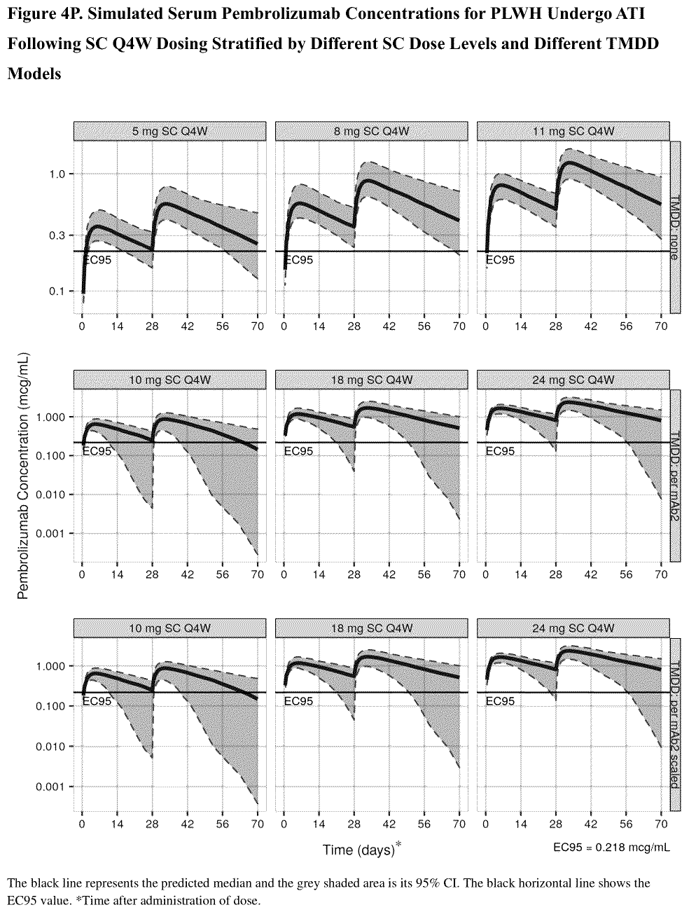

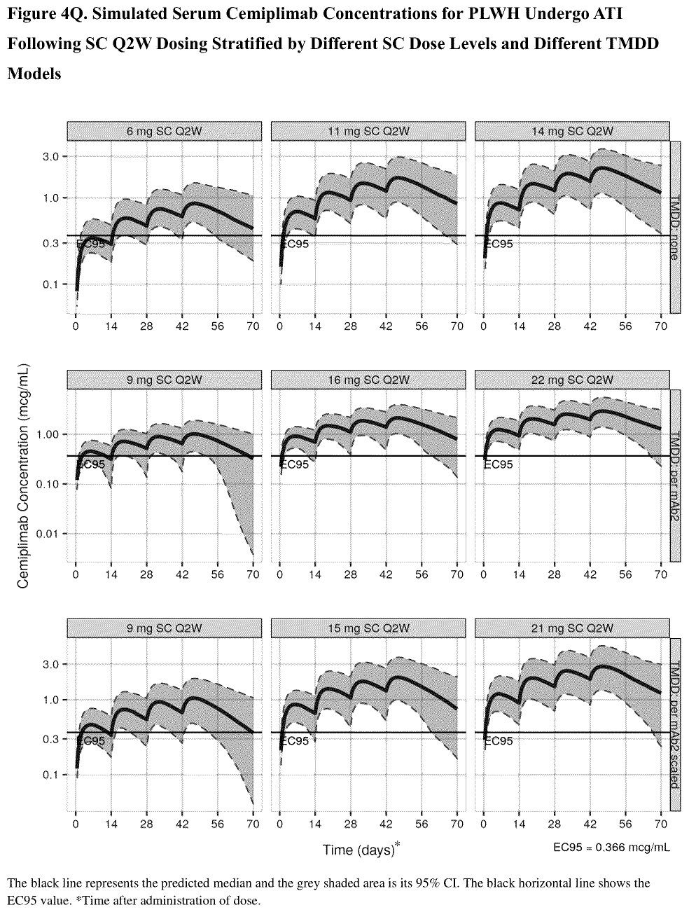

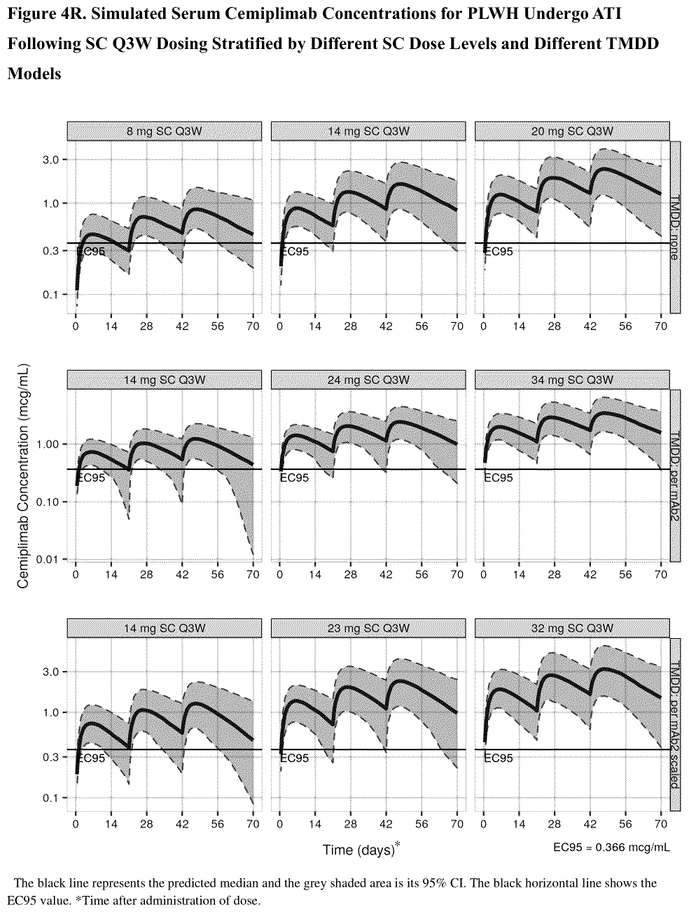

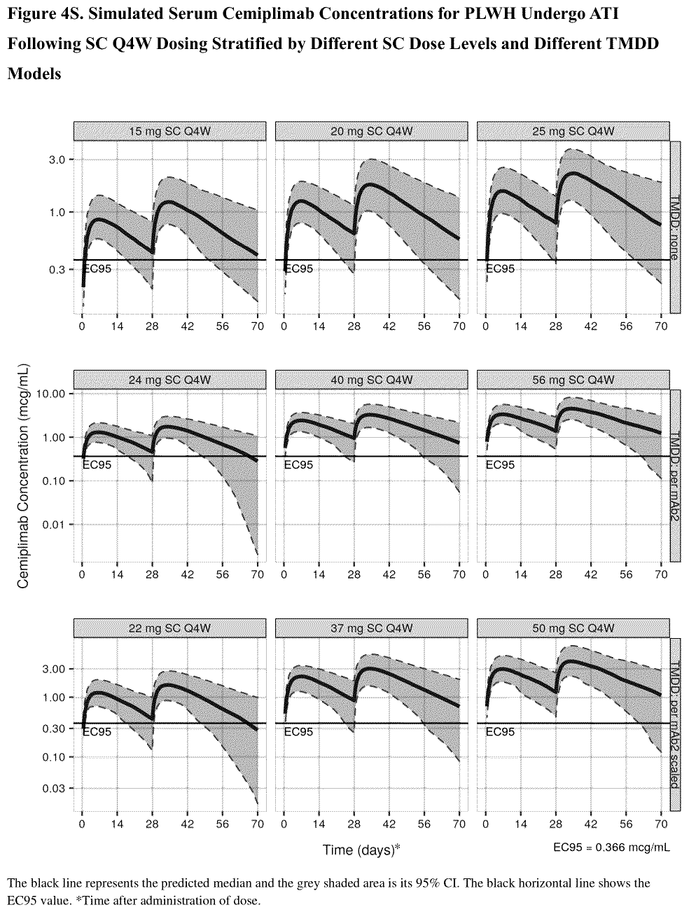

A capacity-limited Eₘₐₓ model with a Hill coefficient was fitted to in-vitro PD-1 receptor saturation data for each mAb. The model fitting for receptor saturation versus mAb concentrations, and a summary of the parameter estimates of the model are available in FIG. 4A and Table 6F, respectively.

TABLE 6F
Parameter Estimates of Emax models based on
In Vitro PD-1 Receptor Saturation Results

| | | EC₅₀ | Hill | |
|---|---|---|---|---|
| | | (mcg/mL) | Factor | EC₉₅ |
| Drug | Type | (95% CI) | (95% CI) | (mcg/mL) |
| mAb2 | CD8/PD-1 | 0.0327 | 1.81 | 0.166 |
| | | (0.0285, 0.0369) | (1.43, 2.18) | |
| Nivolumab | CD8/PD-1 | 0.0552 | 2.30 | 0.198 |
| | | (0.0457, 0.0647) | (1.52, 3.08) | |
| Pembrolizumab | CD8/PD-1 | 0.0359 | 1.63 | 0.218 |
| | | (0.0326, 0.0392) | (1.41, 1.85) | |
| Cemiplimab | CD8/PD-1 | 0.0873 | 2.06 | 0.366 |
| | | (0.0764, 0.0982) | (1.61, 2.50) | |

CI = confidence interval

### PK Simulation Setup

PK simulations (population size=100 and number of replicates=100) were conducted to find the expected minimal efficacious dosing regimen for nivolumab, pembrolizumab and cemiplimab that could saturate PD-1 receptor saturation of peripheral total CD8+ T cells for at least 70 days and for at least 90% of the simulated PLWH undergo analytical treatment interruption (ATI) after the start of first dose, which is expected to subsequently trigger immune-mediated viral load control. The expected minimal efficacious doses were assessed for dosing intervals of every two weeks (Q2W), every three weeks (Q3W) and every four weeks (Q4W) for both intravenous and subcutaneous administrations.

Literature indicated that target mediated drug disposition (TMDD) accounts for a large portion of monoclonal antibody clearance at low concentrations (Liu et al. Protein Cell. 2018 January; 9(1):15-32). The population pharmacokinetic models publicly available for nivolumab, pembrolizumab and cemiplimab were previously developed for cancer patients that were administered high doses leading to higher concentrations where TMDD effect may be saturated. Therefore, to enable extrapolation from cancer patients to PLWH and explore the minimal efficacious dosing regimen at lower doses level for immune-medicated viral load control, different levels of TMDD effects were incorporated into the existed population PK models of nivolumab, pembrolizumab and cemiplimab, assuming:
1. Drug exhibits no TMDD effect
2. Drug exhibits TMDD similar to the estimated effects of mAb2 in people living with HIV
3. Drug exhibits TMDD similar to the estimated effects of mAb2 in people living with HIV, but scaled by the ratio of EC₅₀ of PD-1 saturation of that drug to the EC₅₀ of PD-1 saturation of mAb2

The scaled Michaelis constant (i.e., Kₘ) for nivolumab, pembrolizumab and cemiplimab are shown in Table 6G.

TABLE 6G
Scaled Michaelis Constant (Kₘ) for Nivolumab,
Pembrolizumab and Cemiplimab

| | | EC₅₀ | Scaling | Michaelis Constant, |
|---|---|---|---|---|
| Drug | Type | (mcg/mL) | Factor | Kₘ (mcg/mL) |
| mAb2 | CD8/PD-1 | 0.0327 | — | 0.0774 (estimated) |
| Nivolumab | CD8/PD-1 | 0.0552 | 1.69 | 0.13 |
| Pembrolizumab | CD8/PD-1 | 0.0359 | 1.1 | 0.0846 |
| Cemiplimab | CD8/PD-1 | 0.0873 | 2.67 | 0.206 |

### IV Simulation Results

### Nivolumab

Nivolumab concentrations were simulated using pharmacokinetic parameters shown in Table 6B. The first dose was assumed to be given at the beginning of ATI. Covariates of body weight and eGFR were sampled independently from the Phase 1b studies for mAb2 in people living with HIV. Sex was assumed to be evenly distributed in the simulated population. All simulations were conducted assuming the HIV population to have ECOG performance status=0.

### Q2W

The simulated nivolumab concentrations with Q2W IV dosing are presented in FIG. 4B and Table 6H, respectively.

TABLE 6H
The Expected Minimal Nivolumab Efficacious Dosing
Regimens Following IV Q2W for PLWH Undergo ATI
Across Different Population PK Models

| | | The Estimated % |
|---|---|---|
| | | of Subjects have |
| | Dosing Regimen; Applied | Trough Concentrations |
| Drug | Population PK Model | above EC₉₅ |
| Nivolumab | 5 mg IV Q2W; | 94.0 |
| | TMDD: none | |
| | 8 mg IV Q2W; | 93.0 |
| | TMDD: per mAb2 | |
| | 7 mg IV Q2W; | 91.0 |
| | TMDD: per mAb2 scaled | |

Note:
The expected minimal nivolumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q3W

The simulated nivolumab concentrations with Q3W IV dosing are presented in FIG. 4C and Table 61, respectively.

TABLE 6I
The Expected Minimal Nivolumab Efficacious Dosing
Regimens Following IV Q3W for PLWH Undergo ATI
Across Different Population PK Models

| | | The Estimated % |
|---|---|---|
| | | of Subjects have |
| | Dosing Regimen; Applied | trough Concentrations |
| Drug | Population PK Model | above EC₉₅ |
| Nivolumab | 6 mg IV Q3W; | 93.0 |
| | TMDD: none | |
| | 11 mg IV Q3W; | 92.0 |
| | TMDD: per mAb2 | |
| | 10 mg IV Q3W; | 90.0 |
| | TMDD: per mAb2 scaled | |

Note:
The expected minimal nivolumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q4W

The simulated nivolumab concentrations with Q4W IV dosing are presented in FIG. 4D and Table 6J, respectively.

TABLE 6J
The Expected Minimal Nivolumab Efficacious Dosing
Regimens Following IV Q4W for PLWH Undergo ATI
Across Different Population PK Models

| | | The Estimated % |
|---|---|---|
| | | of Subjects have |
| | Dosing Regimen; Applied | trough Concentrations |
| Drug | Population PK Model | above EC₉₅ |
| Nivolumab | 7 mg IV Q4W; | 90.0 |
| | TMDD: none | |
| | 18 mg IV Q4W; | 91.8 |
| | TMDD: per mAb2 | |
| | 18 mg IV Q4W; | 92.6 |
| | TMDD: per mAb2 scaled | |

Note:
The expected minimal nivolumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Overall Conclusions for Nivolumab PK Simulations Following IV Administration

Model-predicted minimal nivolumab efficacious IV dosing regimens are 5 mg IV Q2W (4 doses), 6 mg IV Q3W (3 doses) and 7 mg IV Q4W (2 doses). This indicates that for Q2W, Q3W, and Q4W dosing intervals, equal and greater than 5 mg, 6 mg or 7 mg, respectively, are expected to trigger immune-mediated viral load control for PLWH undergo ATI.

### Pembrolizumab

Pembrolizumab concentrations were simulated using pharmacokinetic parameters shown in Table 6D. The first dose was assumed to be given at the beginning of ATI. Covariates of body weight and albumin were sampled independently from the Phase 1b studies for mAb2 in people living with HIV. Sex was assumed to be evenly distributed in the simulated population. All simulations were conducted assuming the HIV population to have IgG equal to the reference value as these data were not available in the Phase 1b studies for mAb2 in people living with HIV.

### Q2W

The simulated pembrolizumab concentrations with Q2W IV dosing are presented in FIG. 4E and Table 6K, respectively.

TABLE 6K
The Expected Minimal Pembrolizumab Efficacious
Dosing Regimens Following IV Q2W for PLWH Undergo
ATI Across Different Population PK Models

| | | The Estimated % of |
|---|---|---|
| | | Subjects have |
| | Dosing Regimen; Applied | trough Concentrations |
| Drug | Population PK Model | above EC₉₅ |
| Pembrolizumab | 4 mg IV Q2W; | 100 |
| | TMDD: none | |
| | 7 mg IV Q2W; | 93.0 |
| | TMDD: per mAb2 | |
| | 7 mg IV Q2W; | 93.0 |
| | TMDD: per mAb2 scaled | |

Note:
The expected minimal pembrolizumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q3W

The simulated pembrolizumab concentrations with Q3W IV dosing are presented in FIG. 4F and Table 6L, respectively.

TABLE 6L
The Expected Minimal Pembrolizumab Efficacious
Dosing Regimens Following IV Q3W for PLWH Undergo
ATI Across Different Population PK Models

| | | The Estimated % of |
|---|---|---|
| | | Subjects have |
| | Dosing Regimen; Applied | trough Concentrations |
| Drug | Population PK Model | above EC₉₅ |
| Pembrolizumab | 4 mg IV Q3W; | 94.0 |
| | TMDD: none | |
| | 9 mg IV Q3W; | 90.0 |
| | TMDD: per mAb2 | |
| | 9 mg IV Q3W; | 90.0 |
| | TMDD: per mAb2 scaled | |

Note:
The expected minimal pembrolizumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q4W

The simulated pembrolizumab concentrations with Q4W IV dosing are presented in FIG. 4G and Table 6M, respectively.

TABLE 6M
The Expected Minimal Pembrolizumab Efficacious
Dosing Regimens Following IV Q4W for PLWH Undergo
ATI Across Different Population PK Models

| | | The Estimated % of |
|---|---|---|
| | | Subjects have |
| | Dosing Regimen; Applied | trough Concentrations |
| Drug | Population PK Model | above EC₉₅ |
| Pembrolizumab | 5 mg IV Q4W; | 93.0 |
| | TMDD: none | |
| | 13 mg IV Q4W; | 90.1 |
| | TMDD: per mAb2 | |
| | 13 mg IV Q4W; | 90.4 |
| | TMDD: per mAb2 scaled | |

Note:
The expected minimal pembrolizumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Overall Conclusions for Pembrolizumab PK Simulations Following IV Administration

Model-predicted minimal pembrolizumab efficacious IV dosing regimens are 4 mg IV Q2W (4 doses), 4 mg IV Q3W (3 doses) and 5 mg IV Q4W (2 doses). This indicates that for Q2W, Q3W, and Q4W dosing intervals, equal and greater than 4 mg, 4 mg or 5 mg, respectively, are expected to trigger immune-mediated viral load control for PLWH undergo ATI.

### Cemiplimab

Cemiplimab concentrations were simulated using pharmacokinetic parameters shown in Table 6E. The first dose was assumed to be given at the beginning of ATI. Covariates of alanine aminotransferase and albumin were sampled independently from the Phase 1b studies for mAb2 in people living with HIV. However, due to the observed high correlation between body weight and BMI, the sampling process was modified to ensure that body weight and BMI were sampled dependently, capturing their interdependent relationship. All simulations were conducted assuming the HIV population to have IgG equal to the reference value. In addition, the simulated population was assumed to be non-black, equal to the reference category for the effect of race on T₅₀, as the effect of black race on T₅₀ was estimated to be rather small (6% lower), with the 95% CI including the no effect value. Of note, the cemiplimab pharmacokinetic model for IV dosing includes time-varying clearance, with clearance decreasing over time. Thus, the simulations with assumed TMDD effects account for a reduction in clearance due to lower concentrations (TMDD) as well as increasing time since first dose (time-varying clearance). Q2W

The simulated cemiplimab concentrations with Q2W IV dosing are presented in FIG. 4H and Table 6N, respectively.

TABLE 6N
The Expected Minimal Cemiplimab Efficacious Dosing
Regimens Following IV Q2W for PLWH Undergo ATI
Across Different Population PK Models

| | | The Estimated % of |
|---|---|---|
| | | Subjects have |
| | Dosing Regimen; Applied | trough Concentrations |
| Drug | Population PK Model | above EC₉₅ |
| Cemiplimab | 6 mg IV Q2W; | 91.0 |
| | TMDD: none | |
| | 10 mg IV Q2W; | 95.0 |
| | TMDD: per mAb2 | |
| | 9 mg IV Q2W; | 94.0 |
| | TMDD: per mAb2 scaled | |

Note:
The expected minimal cemiplimab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q3W

The simulated cemiplimab concentrations with Q3W IV dosing are presented in FIG. 4I and Table 6O, respectively.

TABLE 6O
The Expected Minimal Cemiplimab Efficacious Dosing
Regimens Following IV Q3W for PLWH Undergo ATI
Across Different Population PK Models

| | | The Estimated % of |
|---|---|---|
| | | Subjects have |
| | Dosing Regimen; Applied | trough Concentrations |
| Drug | Population PK Model | above EC₉₅ |
| Cemiplimab | 9 mg IV Q3W; | 92.0 |
| | TMDD: none | |
| | 15 mg IV Q3W; | 94.0 |
| | TMDD: per mAb2 | |
| | 14 mg IV Q3W; | 94.0 |
| | TMDD: per mAb2 scaled | |

Note:
The expected minimal cemiplimab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q4W

The simulated cemiplimab concentrations with Q4W IV dosing are presented in FIG. 4J and Table 6P, respectively.

TABLE 6P
The Expected Minimal Cemiplimab Efficacious Dosing
Regimens Following IV Q4W for PLWH Undergo ATI
Across Different Population PK Models

| | | The Estimated % of |
|---|---|---|
| | | Subjects have |
| | Dosing Regimen; Applied | trough Concentrations |
| Drug | Population PK Model | above EC₉₅ |
| Cemiplimab | 12 mg IV Q4W; | 90.8 |
| | TMDD: none | |
| | 23 mg IV Q4W; | 93.0 |
| | TMDD: per mAb2 | |
| | 23 mg IV Q4W; | 93.9 |
| | TMDD: per mAb2 scaled | |

Note:
The expected minimal cemiplimab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Overall Conclusions for Cemiplimab PK Simulations Following IV Administration

Model-predicted minimal cemiplimab efficacious IV dosing regimens are 6 mg IV Q2W (4 doses), 9 mg IV Q3W (3 doses) and 12 mg IV Q4W (2 doses). This indicates that for Q2W, Q3W, and Q4W dosing intervals, equal and greater than 6 mg, 9 mg or 12 mg, respectively, are expected to trigger immune-mediated viral load control for PLWH undergo ATI.

### SC Simulation Results

### Nivolumab

The nivolumab serum concentration following SC administration were simulated using the pharmacokinetic parameters shown in Table 6C. The first dose was assumed to be given at the beginning of ATI. Covariates of body weight and eGFR were sampled independently from the Phase 1b studies of mAb2 in people living with HIV. Sex was assumed to be evenly distributed in the simulated population. All simulations were conducted assuming the HIV population to have oncology covariates set to the reference value, like performance status=0. Since the simulations were conducted for nivolumab monotherapy, the effects of co-administration on bioavailability and absorption factor were not included. All additional covariates, namely performance status, race and cancer type were not included as they were not relevant to the HIV population simulated here. Of note, the nivolumab pharmacokinetic model for SC dosing includes time-varying clearance, with clearance decreasing over time. Thus, the simulations with assumed TMDD effects account for a reduction in clearance due to lower concentrations (TMDD) as well as increasing time since first dose (time-varying clearance).

### Q2W

The simulated nivolumab concentrations with Q2W SC dosing are presented in FIG. 4K and Table 6Q, respectively.

TABLE 6Q
The Expected Minimal Nivolumab Efficacious Dosing
Regimens Following SC Q2W for PLWH Undergo ATI
Across Different Population PK Models

| | | The Estimated % of |
|---|---|---|
| | | Subjects have |
| | Dosing Regimen; Applied | trough Concentrations |
| Drug | Population PK Model | above EC₉₅ |
| Nivolumab | 5 mg SC Q2W; | 91.0 |
| | TMDD: none | |
| | 10 mg SC Q2W; | 91.0 |
| | TMDD: per mAb2 | |
| | 9 mg SC Q2W; | 90.0 |
| | TMDD: per mAb2 scaled | |

Note:
The expected minimal nivolumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q3W

The simulated nivolumab concentrations with Q3W SC dosing are presented in FIG. 4L and Table 6R, respectively.

TABLE 6R
The Expected Minimal Nivolumab Efficacious Dosing
Regimens Following SC Q3W for PLWH Undergo ATI
Across Different Population PK Models

| | Dosing Regimen; | The Estimated % of Subjects |
|---|---|---|
| | Applied Population | have trough Concentrations |
| Drug | PK Model | above EC₉₅ |
| Nivolumab | 7 mg SC Q3W; | 97.0 |
| | TMDD: none | |
| | 14 mg SC Q3W; | 92.0 |
| | TMDD: per mAb2 | |
| | 13 mg SC Q3W; | 92.0 |
| | TMDD: per mAb2 scaled | |

Note:
The expected minimal nivolumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q4W

The simulated nivolumab concentrations with Q4W SC dosing are presented in FIG. 4M and Table 6S, respectively.

TABLE 6S
The Expected Minimal Nivolumab Efficacious Dosing
Regimens Following SC Q4W for PLWH Undergo ATI
Across Different Population PK Models

| | Dosing Regimen; | The Estimated % of Subjects |
|---|---|---|
| | Applied Population | have trough Concentrations |
| Drug | PK Model | above EC₉₅ |
| Nivolumab | 8 mg SC Q4W; | 93.0 |
| | TMDD: none | |
| | 22 mg SC Q4W; | 91.4 |
| | TMDD: per mAb2 | |
| | 21 mg SC Q4W; | 91.7 |
| | TMDD: per mAb2 scaled | |

Note:
The expected minimal nivolumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Overall Conclusions for Nivolumab PK Simulations Following SC Administration

Model-predicted minimal nivolumab efficacious SC dosing regimens are 5 mg SC Q2W (4 doses), 7 mg SC Q3W (3 doses) and 8 mg SC Q4W (2 doses). This indicates that for Q2W, Q3W, and Q4W dosing intervals, equal and greater than 5 mg, 7 mg or 8 mg, respectively, are expected to trigger immune-mediated viral load control for PLWH undergo ATI.

### Pembrolizumab

The pembrolizumab serum concentration following SC administration were simulated using the IV pharmacokinetic parameters shown in Table 6D and pre-defined values for SC bioavailability (F1) and SC absorption rate (Kₐ). The SC bioavailability (F1) was set to 0.640 based on a literature reported value (Jacobs et al. In: Proceedings of the American Association for Cancer Research Annual Meeting 2021; 2021 Apr. 10-15 and May 17-21. Philadelphia (PA): AACR; Cancer Res 2021; 81(13_Suppl): Abstract nr CT143). Because of the missing information of Kₐ, it was set to the mAb2 estimate (0.237 1/day, see Table 6A). The first dose was assumed to be given at the beginning of ATI. Covariates of body weight and albumin were sampled independently from the mAb2 Phase 1b studies in people living with HIV. Sex was assumed to be evenly distributed in the simulated population. All simulations were conducted assuming the HIV population to have IgG equal to the reference value.

### Q2W

The simulated pembrolizumab concentrations with Q2W SC dosing are presented in FIG. 4N and Table 6T, respectively.

TABLE 6T
The Expected Minimal Pembrolizumab Efficacious
Dosing Regimens Following SC Q2W for PLWH Undergo
ATI Across Different Population PK Models

| | Dosing Regimen; | The Estimated % of Subjects |
|---|---|---|
| | Applied Population | have trough Concentrations |
| Drug | PK Model | above EC₉₅ |
| Pembrolizumab | 5 mg SC Q2W; | 100 |
| | TMDD: none | |
| | 9 mg SC Q2W; | 91.0 |
| | TMDD: per mAb2 | |
| | 9 mg SC Q2W; | 91.0 |
| | TMDD: per mAb2 scaled | |

Note:
The expected minimal pembrolizumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q3W

The simulated pembrolizumab concentrations with Q3W SC dosing are presented in FIG. 4O and Table 6U, respectively.

TABLE 6U
The Expected Minimal Pembrolizumab Efficacious
Dosing Regimens Following SC Q3W for PLWH Undergo
ATI Across Different Population PK Models

| | Dosing Regimen; | The Estimated % of Subjects |
|---|---|---|
| | Applied Population | have trough Concentrations |
| Drug | PK Model | above EC₉₅ |
| Pembrolizumab | 6 mg SC Q3W; | 99.0 |
| | TMDD: none | |
| | 12 mg SC Q3W; | 90.0 |
| | TMDD: per mAb2 | |
| | 12 mg SC Q3W; | 90.0 |
| | TMDD: per mAb2 scaled | |

Note:
The expected minimal pembrolizumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q4W

The simulated pembrolizumab concentrations with Q4W SC dosing are presented in FIG. 4P and Table 6V, respectively.

TABLE 6V
The Expected Minimal Pembrolizumab Efficacious
Dosing Regimens Following SC Q4W for PLWH Undergo
ATI Across Different Population PK Models

| | Dosing Regimen; | The Estimated % of Subjects |
|---|---|---|
| | Applied Population | have trough Concentrations |
| Drug | PK Model | above EC₉₅ |
| Pembrolizumab | 8 mg SC Q4W; | 99.1 |
| | TMDD: none | |
| | 18 mg SC Q4W; | 90.1 |
| | TMDD: per mAb2 | |
| | 18 mg SC Q4W; | 90.4 |
| | TMDD: per mAb2 scaled | |

Note:
The expected minimal pembrolizumab efficacious dosing regimen is defined as a dosing regimen which can saturate PD1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immunemediated viral load control;
Trough concentrations = the serum concentration of drug immediately before the next dose is administered;
TMDD = target mediated drug disposition

### Overall Conclusions for Pembrolizumab PK Simulations Following SC Administration

Model predicted minimal pembrolizumab efficacious SC dosing regimens are 5 mg SC Q2W (4 doses), 6 mg SC Q3W (3 doses) and 8 mg SC Q4W (2 doses). This indicates that for Q2W, Q3W, and Q4W dosing intervals, equal and greater than 5 mg, 6 mg or 8 mg, respectively, are expected to trigger immune mediated viral load control for PLWH undergo ATI.

### Cemiplimab

The cemiplimab serum concentration following SC administration were simulated using the IV pharmacokinetic parameters shown in Table 6E and pre-defined values for SC bioavailability (F1) and SC absorption rate (Kₐ). Because of the missing information about both F1 and Kₐ in the literature, both were set to the mAb2 estimates (see Table 6A). The first dose was assumed to be given at the beginning of ATI. Covariates of body weight and BMI were sampled from the Phase 1b studies for mAb2 in people living with HIV accounting for the correlation between them. Other covariates, namely, alanine aminotransferase and albumin were sampled independently from the Phase 1b studies for mAb2 in people living with HIV. Covariate effects were included similar to the IV simulations.

### Q2W

The simulated cemiplimab concentrations with Q2W SC dosing are presented in FIG. 4Q and Table 6W, respectively.

TABLE 6W
The Expected Minimal Cemiplimab Efficacious Dosing
Regimens Following SC Q2W for PLWH Undergo ATI
Across Different Population PK Models

| | Dosing Regimen; | The Estimated % of Subjects |
|---|---|---|
| | Applied Population | have trough Concentrations |
| Drug | PK Model | above EC₉₅ |
| Cemiplimab | 11 mg SC Q2W; | 96.0 |
| | TMDD: none | |
| | 16 mg SC Q2W; | 93.0 |
| | TMDD: per mAb2 | |
| | 15 mg SC Q2W; | 94.0 |
| | TMDD: per mAb2 scaled | |

Note:
The expected minimal cemiplimab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q3W

The simulated cemiplimab concentrations with Q3W SC dosing are presented in FIG. 4R and Table 6X, respectively.

TABLE 6X
The Expected Minimal Cemiplimab Efficacious Dosing
Regimens Following SC Q3W for PLWH Undergo ATI
Across Different Population PK Models

| | Dosing Regimen; | The Estimated % of Subjects |
|---|---|---|
| | Applied Population | have trough Concentrations |
| Drug | PK Model | above EC₉₅ |
| Cemiplimab | 14 mg SC Q3W; | 92.0 |
| | TMDD: none | |
| | 24 mg SC Q3W; | 93.0 |
| | TMDD: per mAb2 | |
| | 23 mg SC Q3W; | 94.0 |
| | TMDD: per mAb2 scaled | |

Note:
The expected minimal cemiplimab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control;
Trough concentrations = the serum concentration of drug immediately before the next dose is administered;
TMDD = target mediated drug disposition

### Q4W

The simulated cemiplimab concentrations with Q4W SC dosing are presented in FIG. 4S and Table 6Y, respectively.

TABLE 6Y
The Expected Minimal Cemiplimab Efficacious Dosing
Regimens Following SC Q4W for PLWH Undergo ATI
Across Different Population PK Models

| | Dosing Regimen; | The Estimated % of Subjects |
|---|---|---|
| | Applied Population | have trough Concentrations |
| Drug | PK Model | above EC₉₅ |
| Cemiplimab | 20 mg SC Q4W; | 91.3 |
| | TMDD: none | |
| | 40 mg SC Q4W; | 93.8 |
| | TMDD: per mAb2 | |
| | 37 mg SC Q4W; | 93.6 |
| | TMDD: per mAb2 scaled | |

Note:
The expected minimal cemiplimab efficacious dosing regimen is defined as a dosing regimen which can saturate PD1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immunemediated viral load control;
Trough concentrations = the serum concentration of drug immediately before the next dose is administered;
TMDD = target mediated drug disposition

### Overall Conclusions for Cemiplimab PK Simulations Following SC Administration

Model predicted minimal cemiplimab efficacious SC dosing regimens are 11 mg SC Q2W (4 doses), 14 mg SC Q3W (3 doses) and 20 mg SC Q4W (2 doses). This indicates that for Q2W, Q3W, and Q4W dosing intervals, equal and greater than 11 mg, 14 mg or 20 mg, respectively, are expected to trigger immune mediated viral load control for PLWH undergo ATI.

### Sensitivity Analysis

Additional analyses were conducted to establish sensitivity of the minimal SC efficacious dose, considering the uncertainty around absorption rate (Kₐ) and bioavailability (F1).

### Nivolumab

### Low SC Bioavailability (F1)

Assuming SC bioavailability is equal to the worst-case of 60% per literature (Zhao et al. Clin Pharmacol Ther, 2024:115: 488-497), serum concentrations were simulated for various subcutaneous nivolumab dosing regimens. The minimal efficacious doses for those various dosing regimens and their TMDD assumptions are presented in Table 6Z.

TABLE 6Z
The Expected Minimal Nivolumab Efficacious Dosing
Regimens Following SC Q2W, Q3W and Q4W with Worst
Case Bioavailability of 60.6% for PLWH Undergo
ATI Across Different Population PK Models

| | Dosing Regimen; | The Estimated % of Subjects |
|---|---|---|
| | Applied Population | have trough Concentrations |
| Drug | PK Model | above EC₉₅ |
| Nivolumab | 8 mg SC Q2W; | 94.0 |
| | TMDD: none | |
| | 13 mg SC Q2W; | 94.0 |
| | TMDD: per mAb2 | |
| | 12 mg SC Q2W; | 94.0 |
| | TMDD: per mAb2 scaled | |
| | 9 mg SC Q3W; | 92.0 |
| | TMDD: none | |
| | 18 mg SC Q3W; | 92.0 |
| | TMDD: per mAb2 | |
| | 16 mg SC Q3W; | 90.0 |
| | TMDD: per mAb2 scaled | |
| | 11 mg SC Q4W; | 92.0 |
| | TMDD: none | |
| | 28 mg SC Q4W; | 90.4 |
| | TMDD: per mAb2 | |
| | 26 mg SC Q4W; | 90.4 |
| | TMDD: per mAb2 scaled | |

Note:
The expected minimal nivolumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control; Trough concentrations = the serum concentration of drug immediately before the next dose is administered; TMDD = target mediated drug disposition; In these simulations, the worst-case SC bioavailability of 0.606 was assumed.

### Pembrolizumab

### Low SC Absorption Rate (Kₐ)

Assuming SC absorption rate is equal to 0.168 (1/day), the lower bound of 95% CI of the estimated mAb2 Kₐ (Table 6A), serum concentrations were simulated for various subcutaneous pembrolizumab dosing regimens. The minimum efficacious doses were determined based on these simulations (Table 6ZA).

TABLE 6ZA
The Expected Minimal Pembrolizumab Efficacious
Dosing Regimens Following SC Q2W, Q3W and Q4W
with Low SC Absorption Rate for PLWH Undergo
ATI Across Different Population PK Models

| | Dosing Regimen; | The Estimated % of Subjects |
|---|---|---|
| | Applied Population | have trough Concentrations |
| Drug | PK Model | above EC₉₅ |
| Pembrolizumab | 5 mg SC Q2W; | 99.0 |
| | TMDD: none | |
| | 9 mg SC Q2W; | 92.0 |
| | TMDD: per mAb2 | |
| | 9 mg SC Q2W; | 92.0 |
| | TMDD: per mAb2 scaled | |
| | 6 mg SC Q3W; | 99.0 |
| | TMDD: none | |
| | 12 mg SC Q3W; | 90.0 |
| | TMDD: per mAb2 | |
| | 12 mg SC Q3W; | 90.0 |
| | TMDD: per mAb2 scaled | |
| | 6 mg SC Q4W; | 93.0 |
| | TMDD: none | |
| | 17 mg SC Q4W; | 90.1 |
| | TMDD: per mAb2 | |
| | 17 mg SC Q4W; | 90.4 |
| | TMDD: per mAb2 scaled | |

Note:
The expected minimal pembrolizumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control; Trough concentrations = the serum concentration of drug immediately before the next dose is administered; TMDD = target mediated drug disposition; In these simulations, Kₐ of 0.168 (1/day) was assumed.

### High SC Absorption Rate (Kₐ)

Assuming SC absorption rate is equal to 0.334 (1/day), the upper bound of 95% CI of the estimated mAb2 Kₐ (Table 6A), serum concentrations were simulated for various subcutaneous pembrolizumab dosing regimens. The minimum efficacious doses were determined based on these simulations (Table 6ZB).

TABLE 6ZB
The Expected Minimal Pembrolizumab Efficacious
Dosing Regimens Following SC Q2W, Q3W and Q4W
with High SC Absorption Rate for PLWH Undergo
ATI Across Different Population PK Models

| | Dosing Regimen; | The Estimated % of Subjects |
|---|---|---|
| | Applied Population | have trough Concentrations |
| Drug | PK Model | above EC₉₅ |
| Pembrolizumab | 5 mg SC Q2W; | 100 |
| | TMDD: none | |
| | 9 mg SC Q2W; | 91.0 |
| | TMDD: per mAb2 | |
| | 9 mg SC Q2W; | 91.0 |
| | TMDD: per mAb2 scaled | |
| | 6 mg SC Q3W; | 99.0 |
| | TMDD: none | |
| | 13 mg SC Q3W; | 91.0 |
| | TMDD: per mAb2 | |
| | 13 mg SC Q3W; | 91.0 |
| | TMDD: per mAb2 scaled | |
| | 7 mg SC Q4W; | 95.0 |
| | TMDD: none | |
| | 20 mg SC Q4W; | 90.7 |
| | TMDD: per mAb2 | |
| | 20 mg SC Q4W; | 90.8 |
| | TMDD: per mAb2 scaled | |

Note:
The expected minimal pembrolizumab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control; Trough concentrations = the serum concentration of drug immediately before the next dose is administered; TMDD = target mediated drug disposition; In these simulations, Kₐ of 0.334 (1/day) was assumed.

### Cemiplimab

### Low SC Absorption Rate (Kₐ)

Assuming SC absorption rate is equal to 0.168 (1/day), the lower bound of 95% CI of the estimated mAb2 Kₐ (Table 6A), serum concentrations were simulated following various subcutaneous cemiplimab dosing regimens. The minimum efficacious doses were determined based on these simulations (Table 6ZC).

TABLE 6ZC
The Expected Minimal Cemiplimab Efficacious Dosing Regimens
Following SC Q2W, Q3W and Q4W with Low SC Absorption Rate
for PLWH Undergo ATI Across Different Population PK Models

| | Dosing Regimen; | The Estimated % of Subjects |
|---|---|---|
| | Applied Population | have trough Concentrations |
| Drug | PK Model | above EC₉₅ |
| Cemiplimab | 10 mg SC Q2W; | 90.0 |
| | TMDD: none | |
| | 14 mg SC Q2W; | 90.0 |
| | TMDD: per mAb2 | |
| | 14 mg SC Q2W; | 94.0 |
| | TMDD: per mAb2 scaled | |
| | 14 mg SC Q3W; | 93.0 |
| | TMDD: none | |
| | 21 mg SC Q3W; | 93.0 |
| | TMDD: per mAb2 | |
| | 19 mg SC Q3W; | 90.0 |
| | TMDD: per mAb2 scaled | |
| | 18 mg SC Q4W; | 90.0 |
| | TMDD: none | |
| | 32 mg SC Q4W; | 90.2 |
| | TMDD: per mAb2 | |
| | 31 mg SC Q4W; | 91.1 |
| | TMDD: per mAb2 scaled | |

Note:
The expected minimal cemiplimab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control; Trough concentrations = the serum concentration of drug immediately before the next dose is administered; TMDD = target mediated drug disposition; In these simulations, Kₐ of 0.168 (1/day) was assumed.

### High SC Absorption Rate (Kₐ)

Assuming SC absorption rate is equal to 0.334 (1/day), the upper bound of 95% CI of the estimated mAb2 Kₐ (Table 6A), serum concentrations were simulated following various subcutaneous cemiplimab dosing regimens. The minimum efficacious doses were determined based on these simulations (Table 6ZD).

TABLE 6ZD
The Expected Minimal Cemiplimab Efficacious Dosing Regimens
Following SC Q2W, Q3W and Q4W with High SC Absorption Rate
for PLWH Undergo ATI Across Different Population PK Models

| | Dosing Regimen; | The Estimated % of Subjects |
|---|---|---|
| | Applied Population | have trough Concentrations |
| Drug | PK Model | above EC₉₅ |
| Cemiplimab | 11 mg SC Q2W; | 92.0 |
| | TMDD: none | |
| | 15 mg SC Q2W; | 91.0 |
| | TMDD: per mAb2 | |
| | 14 mg SC Q2W; | 93.0 |
| | TMDD: per mAb2 scaled | |
| | 15 mg SC Q3W; | 90.0 |
| | TMDD: none | |
| | 23 mg SC Q3W; | 91.0 |
| | TMDD: per mAb2 | |
| | 22 mg SC Q3W; | 94.0 |
| | TMDD: per mAb2 scaled | |
| | 23 mg SC Q4W; | 93.4 |
| | TMDD: none | |
| | 38 mg SC Q4W; | 90.1 |
| | TMDD: per mAb2 | |
| | 36 mg SC Q4W; | 91.5 |
| | TMDD: per mAb2 scaled | |

Note:
The expected minimal cemiplimab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control; Trough concentrations = the serum concentration of drug immediately before the next dose is administered; TMDD = target mediated drug disposition; In these simulations, Kₐ of 0.334 (1/day) was assumed.

### Low SC Bioavailability (F1)

Assuming SC bioavailability is equal to 0.383, the lower bound of 95% CI of the estimated mAb2 F1 (Table 6A), serum concentrations were simulated following various subcutaneous cemiplimab dosing regimens. The minimum efficacious doses were determined based on these simulations (Table 6ZE).

TABLE 6ZE
The Expected Minimal Cemiplimab Efficacious Dosing Regimens
Following SC Q2W, Q3W and Q4W with Low Bioavailability for
PLWH Undergo ATI Across Different Population PK Models

| | Dosing Regimen; | The Estimated % of Subjects |
|---|---|---|
| | Applied Population | have trough Concentrations |
| Drug | PK Model | above EC₉₅ |
| Cemiplimab | 13 mg SC Q2W; | 92.0 |
| | TMDD: none | |
| | 18 mg SC Q2W; | 91.0 |
| | TMDD: per mAb2 | |
| | 18 mg SC Q2W; | 94.0 |
| | TMDD: per mAb2 scaled | |
| | 18 mg SC Q3W; | 93.0 |
| | TMDD: none | |
| | 27 mg SC Q3W; | 92.0 |
| | TMDD: per mAb2 | |
| | 25 mg SC Q3W; | 91.0 |
| | TMDD: per mAb2 scaled | |
| | 24 mg SC Q4W; | 90.0 |
| | TMDD: none | |
| | 44 mg SC Q4W; | 90.8 |
| | TMDD: per mAb2 | |
| | 41 mg SC Q4W; | 90.1 |
| | TMDD: per mAb2 scaled | |

Note:
The expected minimal cemiplimab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control; Trough concentrations = the serum concentration of drug immediately before the next dose is administered; TMDD = target mediated drug disposition; In these simulations, F1 of 0.383 was assumed.

### High SC Bioavailability (F1)

Assuming SC bioavailability is equal to 0.603, the upper bound of 95% of the estimated mAb2 F1 (Table 6A), serum concentrations were simulated following various subcutaneous cemiplimab dosing regimens. The minimum efficacious doses were determined based on these simulations (Table 6ZF).

TABLE 6ZF
The Expected Minimal Cemiplimab Efficacious Dosing Regimens
Following SC Q2W, Q3W and Q4W with High Bioavailability for
PLWH Undergo ATI Across Different Population PK Models

| | Dosing Regimen; | The Estimated % of Subjects |
|---|---|---|
| | Applied Population | have trough Concentrations |
| Drug | PK Model | above EC₉₅ |
| Cemiplimab | 8 mg SC Q2W; | 91.0 |
| | TMDD: none | |
| | 12 mg SC Q2W; | 91.0 |
| | TMDD: per mAb2 | |
| | 11 mg SC Q2W; | 91.0 |
| | TMDD: per mAb2 scaled | |
| | 12 mg SC Q3W; | 92.0 |
| | TMDD: none | |
| | 17 mg SC Q3W; | 90.0 |
| | TMDD: per mAb2 | |
| | 16 mg SC Q3W; | 91.0 |
| | TMDD: per mAb2 scaled | |
| | 16 mg SC Q4W; | 90.0 |
| | TMDD: none | |
| | 29 mg SC Q4W; | 90.4 |
| | TMDD: per mAb2 | |
| | 28 mg SC Q4W; | 92.1 |
| | TMDD: per mAb2 scaled | |

Note:
The expected minimal cemiplimab efficacious dosing regimen is defined as a dosing regimen which can saturate PD-1 receptor of peripheral total CD8+ T cells (i.e., serum concentrations > estimated mean EC₉₅) for at least 70 days and for at least 90% of the simulated PLWH undergo ATI after the start of first dose, which is expected to subsequently trigger immune-mediated viral load control; Trough concentrations = the serum concentration of drug immediately before the next dose is administered; TMDD = target mediated drug disposition; In these simulations, F1 of 0.603 was assumed.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof. All publications, patents, and patent applications cited herein are hereby incorporated by reference in their entirety for all purposes.

SEQUENCE LISTING TABLE

| SEQ | | |
|---|---|---|
| ID NO: | Description | Sequence |
| 1 | mAb2 VH CDR#1 | GYTFTHYGMN |
| 2 | mAb2 VH CDR#2 | WVNTYTGEPTYADDFKG |
| 3 | mAb2 VH CDR#3 | EGEGLGFGD |
| 4 | mAb2 VL CDR#1 | RSSQSIVHSHGDTYLE |
| 5 | mAb2 VL CDR#2 | KVSNRFS |
| 6 | mAb2 VL CDR#3 | FQGSHIPVT |
| 7 | mAb2 Heavy Chain | EIQLVQSGAEVKKPGSSVKVSCKASGYTFTHYGM |
| | Variable Region | NWVRQAPGQGLEWVGWVNTYTGEPTYADDFKG |
| | | RLTFTLDTSTSTAYMELSSLRSEDTAVYYCTREGE |
| | | GLGFGDWGQGTTVTVSS |
| 8 | mAb2 Light Chain | DVVMTQSPLSLPVTPGEPASISCRSSQSIVHSHGDT |
| | Variable Region | YLEWYLQKPGQSPQLLIYKVSNRFSGVPDRFSGSG |
| | | SGTDFTLKISRVEAEDVGVYYCFQGSHIPVTFGQG |
| | | TKLEIK |
| 9 | mAb2 Heavy Chain | EIQLVQSGAEVKKPGSSVKVSCKASGYTFTHYGM |
| | | NWVRQAPGQGLEWVGWVNTYTGEPTYADDFKG |
| | | RLTFTLDTSTSTAYMELSSLRSEDTAVYYCTREGE |
| | | GLGFGDWGQGTTVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNHK |
| | | PSNTKVDKKVEPKSCDKTHTCPPCPAPEAAGGPSV |
| | | FLFPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALPAPIEKTISKAKGQP |
| | | REPQVYTLPPSREEMTKNQVSLTCLVKGFYPSDIA |
| | | VEWESNGQPENNYKTTPPVLDSDGSFFLYSKLTVD |
| | | KSRWQQGNVFSCSVMHEALHNHYTQKSLSLSPGK |
| 10 | mAb2 Light Chain | DVVMTQSPLSLPVTPGEPASISCRSSQSIVHSHGDT |
| | | YLEWYLQKPGQSPQLLIYKVSNRFSGVPDRFSGSG |
| | | SGTDFTLKISRVEAEDVGVYYCFQGSHIPVTFGQG |
| | | TKLEIKRTVAAPSVFIFPPSDEQLKSGTASVVCLLN |
| | | NFYPREAKVQWKVDNALQSGNSQESVTEQDSKDS |
| | | TYSLSSTLTLSKADYEKHKVYACEVTHQGLSSPVT |
| | | KSFNRGEC |
| 11 | mAb1 VH CDR#1 | GFNIKNTYMH |
| 12 | mAb1 VH CDR#2 | RIDPAKGHTEYAPKFLG |
| | mAb1 VH CDR#3 | VDV |
| 13 | mAb1 VL CDR#1 | HASQDISDNIG |
| 14 | mAb1 VL CDR#2 | HGTNLED |
| 15 | mAb1 VL CDR#3 | VQYAQFPWT |
| 16 | mAb1 Heavy Chain | EVQLVQSGAEVKKPGSSVKVSCKASGFNIKNTYM |
| | Variable Region | HWVRQAPGQGLEWIGRIDPAKGHTEYAPKFLGRV |
| | | TITADESTNTAYMELSSLRSEDTAVYYCYYVDVW |
| | | GQGTTVTVSS |
| 17 | mAb1 Light Chain | DIQMTQSPSSLSASVGDRVTITCHASQDISDNIGWL |
| | Variable Region | QQKPGKSFKLLIYHGTNLEDGVPSRFSGSGSGTDY |
| | | TLTISSLQPEDFATYYCVQYAQFPWTFGGGTKVEI |
| | | K |
| 18 | mAb1 Heavy Chain | EVQLVQSGAEVKKPGSSVKVSCKASGFNIKNTYM |
| | | HWVRQAPGQGLEWIGRIDPAKGHTEYAPKFLGRV |
| | | TITADESTNTAYMELSSLRSEDTAVYYCYYVDVW |
| | | GQGTTVTVSSASTKGPSVFPLAPSSKSTSGGTAALG |
| | | CLVKDYFPEPVTVSWNSGALTSGVHTFPAVLQSSG |
| | | LYSLSSVVTVPSSSLGTQTYICNVNHKPSNTKVDK |
| | | KVEPKSCDKTHTCPPCPAPELLGGPSVFLFPPKPKD |
| | | TLMISRTPEVTCVVVDVSHEDPEVKFNWYVDGVE |
| | | VHNAKTKPREEQYNSTYRVVSVLTVLHQDWLNG |
| | | KEYKCKVSNKALPAPIEKTISKAKGQPREPQVYTL |
| | | PPSREEMTKNQVSLTCLVKGFYPSDIAVEWESNGQ |
| | | PENNYKTTPPVLDSDGSFFLYSKLTVDKSRWQQG |
| | | NVFSCSVMHEALHNHYTQKSLSLSPGK |
| 19 | mAb1 Light Chain | DIQMTQSPSSLSASVGDRVTITCHASQDISDNIGWL |
| | | QQKPGKSFKLLIYHGTNLEDGVPSRFSGSGSGTDY |
| | | TLTISSLQPEDFATYYCVQYAQFPWTFGGGTKVEI |
| | | KRTVAAPSVFIFPPSDEQLKSGTASVVCLLNNFYPR |
| | | EAKVQWKVDNALQSGNSQESVTEQDSKDSTYSLS |
| | | STLTLSKADYEKHKVYACEVTHQGLSSPVTKSFNR |
| | | GEC |
| 20 | mAb2 truncated | EIQLVQSGAEVKKPGSSVKVSCKASGYTFTHYGM |
| | Heavy Chain | NWVRQAPGQGLEWVGWVNTYTGEPTYADDFKG |
| | | RLTFTLDTSTSTAYMELSSLRSEDTAVYYCTREGE |
| | | GLGFGDWGQGTTVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNHK |
| | | PSNTKVDKKVEPKSCDKTHTCPPCPAPEAAGGPSV |
| | | FLFPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALPAPIEKTISKAKGQP |
| | | REPQVYTLPPSREEMTKNQVSLTCLVKGFYPSDIA |
| | | VEWESNGQPENNYKTTPPVLDSDGSFFLYSKLTVD |
| | | KSRWQQGNVFSCSVMHEALHNHYTQKSLSLSPG |
| 21 | mAb1 truncated | EVQLVQSGAEVKKPGSSVKVSCKASGFNIKNTYM |
| | Heavy Chain | HWVRQAPGQGLEWIGRIDPAKGHTEYAPKFLGRV |
| | | TITADESTNTAYMELSSLRSEDTAVYYCYYVDVW |
| | | GQGTTVTVSSASTKGPSVFPLAPSSKSTSGGTAALG |
| | | CLVKDYFPEPVTVSWNSGALTSGVHTFPAVLQSSG |
| | | LYSLSSVVTVPSSSLGTQTYICNVNHKPSNTKVDK |
| | | KVEPKSCDKTHTCPPCPAPELLGGPSVFLFPPKPKD |
| | | TLMISRTPEVTCVVVDVSHEDPEVKFNWYVDGVE |
| | | VHNAKTKPREEQYNSTYRVVSVLTVLHQDWLNG |
| | | KEYKCKVSNKALPAPIEKTISKAKGQPREPQVYTL |
| | | PPSREEMTKNQVSLTCLVKGFYPSDIAVEWESNGQ |
| | | PENNYKTTPPVLDSDGSFFLYSKLTVDKSRWQQG |
| | | NVFSCSVMHEALHNHYTQKSLSLSPG |

CDRs listed in the table above are based on the Kabat numbering scheme

What is claimed:

1. A method of treating human immunodeficiency virus (HIV) infection in a patient infected with HIV-1, the method comprising administering to the patient a therapeutically effective amount of a monoclonal antibody which binds to human programmed death receptor 1 (PD-1),
wherein the monoclonal antibody which binds to PD-1 is mAb2 which comprises (i) a heavy chain variable region (VH) comprising three CDRs: VH CDR #1, VH CDR #2, and VH CDR #3; and
(ii) a light chain variable region (VL) comprising three CDRs: VL CDR #1, VL CDR #2, and VL CDR #3, wherein:
VH CDR#1 is
(SEQ ID NO: 1)
GYTFTHYGMN;
VH CDR#2 is
(SEQ ID NO: 2)
WVNTYTGEPTYADDFKG;
VH CDR#3 is
(SEQ ID NO: 3)
EGEGLGFGD;
VL CDR#1 is
(SEQ ID NO: 4)
RSSQSIVHSHGDTYLE;
VL CDR#2 is
(SEQ ID NO: 5)
KVSNRFS;
and
VL CDR#3 is
(SEQ ID NO: 6)
FQGSHIPVT.

2.-3. (canceled)

4. The method of claim 1, wherein the mAb2 comprises (i) a heavy chain (HC) comprising the amino acid sequence of SEQ ID NO: 9 or SEQ ID NO: 20; and (ii) a light chain (LC) comprising the amino acid sequence of SEQ ID NO: 10.

5. (canceled)

6. The method of claim 1, wherein the mAb2 is administered to the patient in an amount of about 10 mg or about 20 mg.

7.-9. (canceled)

10. The method of claim 1, wherein the mAb2 is administered to the patient via IV infusion in an amount of about 10 mg at Week 0 (Day 1), Week 2 (Day 15), Week 4 (Day 29), and Week 6 (Day 43).

11. The method of claim 1, wherein the mAb2 is administered to the patient via SC injection in an amount of about 20 mg at Week 0 (Day 1), Week 2 (Day 15), Week 4 (Day 29), and Week 6 (Day 43).

12. The method of claim 1, wherein after about 25 weeks the patient achieves an HIV viral load of less than about 5,000 copies/mL without antiretroviral treatment.

13.-17. (canceled)

18. A method of treating human immunodeficiency virus (HIV) infection in a patient infected with HIV-1, the method comprising administering to the patient a therapeutically effective amount of a monoclonal antibody which binds to human α4β7,
wherein the monoclonal antibody which binds to α4β7 is mAb1 which comprises i) a heavy chain variable region (VH) comprising three CDRs: VH CDR #1, VH CDR #2, and VH VDR #3; and (ii) a light chain variable region (VL) comprising three CDRs: VL CDR #1, VL CDR #2, and VL VDR #3, wherein:
VH CDR#1 is
(SEQ ID NO: 11)
GFNIKNTYMH;
VH CDR#2 is
(SEQ ID NO: 12)
RIDPAKGHTEYAPKFLG;
VH CDR#3 is
VDV;
VL CDR#1 is
(SEQ ID NO: 13)
HASQDISDNIG;
VL CDR#2 is
(SEQ ID NO: 14)
HGTNLED;
and
VL CDR#3 is
(SEQ ID NO: 15)
VQYAQFPWT.

19.-20. (canceled)

21. The method of claim 18, wherein the mAb1 comprises (i) a heavy chain (HC) comprising the amino acid sequence of SEQ ID NO: 18 or SEQ ID NO: 21; and (ii) a light chain (LC) comprising the amino acid sequence of SEQ ID NO: 19.

22. (canceled)

23. The method of claim 18, wherein the mAb1 is administered in an amount of about 800 mg or about 1600 mg.

24.-26. (canceled)

27. The method of claim 18, wherein the mAb1 is administered via IV infusion in an amount of about 800 mg or about 1600 mg at Week 0 (Day 1), Week 4 (Day 29), and Week 8 (Day 57).

28. (canceled)

29. The method of claim 18, wherein after about 25 weeks the patient achieves an HIV viral load of less than about 5,000 copies/mL without antiretroviral treatment.

30.-34. (canceled)

35. A method of treating human immunodeficiency virus (HIV) infection in a patient infected with HIV-1, the method comprising:
administering to the patient a therapeutically effective amount of a first monoclonal antibody which binds to human PD-1; and
administering to the patient a therapeutically effective amount of a second monoclonal antibody which binds to human α4β7.

36. The method of claim 35, wherein the first monoclonal antibody is mAb2 which comprises (i) a VH comprising three CDRs: VH CDR #1, VH CDR #2, and VH CDR #3; and (ii) a VL comprising three CDRs: VL CDR #1, VL CDR #2, and VL CDR #3, wherein:
VH CDR#1 is
(SEQ ID NO: 1)
GYTFTHYGMN;
VH CDR#2 is
(SEQ ID NO: 2)
WVNTYTGEPTYADDFKG;
VH CDR#3 is
(SEQ ID NO: 3)
EGEGLGFGD;
VL CDR#1 is
(SEQ ID NO: 4)
RSSQSIVHSHGDTYLE;
VL CDR#2 is
(SEQ ID NO: 5)
KVSNRFS;
and
VL CDR#3 is
(SEQ ID NO: 6)
FQGSHIPVT.

37. (canceled)

38. The method of claim 36, wherein the mAb2 comprises (i) a heavy chain (HC) comprising the amino acid sequence of SEQ ID NO: 9 or SEQ ID NO: 20; and (ii) a light chain (LC) comprising the amino acid sequence of SEQ ID NO: 10.

39. (canceled)

40. The method of claim 35, wherein the second monoclonal antibody is mAb1 which comprises i) a VH comprising three CDRs: VH CDR #1, VH CDR #2, and VH CDR #3; and (ii) a VL comprising three CDRs: VL CDR #1, VL CDR #2, and VL CDR #3, wherein:
VH CDR#1 is
(SEQ ID NO: 11)
GFNIKNTYMH;
VH CDR#2 is
(SEQ ID NO: 12)
RIDPAKGHTEYAPKFLG;
VH CDR#3 is
VDV;
VL CDR#1 is
(SEQ ID NO: 13)
HASQDISDNIG;
VL CDR#2 is
(SEQ ID NO: 14)
HGTNLED;
and
VL CDR#3 is
(SEQ ID NO: 15)
VQYAQFPWT.

41. (canceled)

42. The method of claim 40, wherein the mAb1 comprises (i) a heavy chain (HC) comprising the amino acid sequence of SEQ ID NO: 18 or SEQ ID NO: 21; and (ii) a light chain (LC) comprising the amino acid sequence of SEQ ID NO: 19.

43.-45. (canceled)

46. The method of claim 35, wherein the first monoclonal antibody is administered in an amount of about 10 mg or about 20 mg.

47. (canceled)

48. The method of claim 35, wherein the second monoclonal antibody is administered in an amount of about 800 mg or about 1600 mg.

49.-51. (canceled)

52. The method of claim 35, wherein the first monoclonal antibody is administered once every 2 weeks for about 6 weeks.

53. The method of claim 35, wherein the second monoclonal antibody is administered once every 4 weeks for about 8 weeks.

54. (canceled)

55. The method of claim 35, wherein the method comprises:
administering the first monoclonal antibody via IV infusion in an amount of about 10 mg on Week 0 (Day 1), Week 2 (Day 15), Week 4 (Day 29), and Week 6 (Day 43); and
administering the second monoclonal antibody via IV infusion in an amount of about 800 mg or about 1600 mg on Week 0 (Day 1), Week 4 (Day 29), and Week 8 (Day 57).

56. (canceled)

57. The method of claim 35, wherein the method comprises:
administering the first monoclonal antibody via SC injection in an amount of about 20 mg on Week 0 (Day 1), Week 2 (Day 15), Week 4 (Day 29), and Week 6 (Day 43); and
administering the second monoclonal antibody via IV infusion in an amount of about 800 mg or about 1600 mg on Week 0 (Day 1), Week 4 (Day 29), and Week 8 (Day 57).

58. (canceled)

59. The method of claim 35, wherein (1) the time to viral rebound following interruption of antiretroviral treatment is increased; and/or (2) the peak viral load at viral rebound before starting antiretroviral treatment is decreased, compared with treatment prior to the administration of both anti-PD-1 and anti-α4β7 antibodies, wherein the viral rebound is indicated by a viral load greater than about 1000 copies/mL.

60. (canceled)

61. The method of claim 35, wherein after about 25 weeks the patient achieves an HIV viral load of less than about 5,000 copies/mL without antiretroviral treatment.

62.-69. (canceled)
